(19) United States
(12) Patent Application Publication
(10) Pub. No.: US 2009/0202517 A1
(43) Pub. Date: Aug. 13, 2009

(54) AGENTS FOR IMPROVING INFLAMMATORY BOWEL DISEASE

(76) Inventors: Hiroyuki Yoneyama, Tokyo (JP); Kyoko Wakamatsu, Tokyo (JP)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/576,040

(22) PCT Filed: Nov. 28, 2006
(86) PCT No.: PCT/JP2006/323673

§ 371 Date: Mar. 5, 2008

(30) Foreign Application Priority Data
Aug. 17, 2006 (JP) 2006-222238

(51) Int. Cl. A61K 38/48; C12N 9/50; C12Q 1/37

(52) U.S. Cl. 424/94.67; 435/219; 435/23

(57) ABSTRACT

The present inventors discovered for the first time that intestinal inflammation could be efficiently suppressed by suppressing the production or accumulation of chondroitin sulfate proteoglycans. Specifically, inflammation in the large intestine can be suppressed by using siRNA to suppress the expression of versican, which is one of the chondroitin sulfate proteoglycans. Compounds used as siRNA, such as nucleic acids, can be used as effective agents for suppressing intestinal inflammation. Furthermore, the above finding also suggests that such intestinal inflammation-suppressing agents can be found by screening for compounds that suppress the production or accumulation of chondroitin sulfate proteoglycans.

### TECHNICAL FIELD

The present invention relates to agents for treating or preventing inflammatory bowel diseases (IBD) such as ulcerative colitis, and uses thereof.

### BACKGROUND ART

Inflammatory bowel disease (IBD) is a general name for a group of chronic intractable diseases that lead to inflammatory lesions in the small intestine and large intestine. The representative diseases are ulcerative colitis (UC) and Crohn's disease. Ulcerative colitis is a nonspecific diffuse inflammation which causes erosions and ulcers in the mucosa and submucosa of the large intestine. The disease develops in the lower part of the large intestine first, and in most cases, either the lesion is confined to the site (left-sided colitis) or it spreads ascendingly and affects the entire large intestine (total colitis). The characteristic features are lead pipe-like appearance of the large intestine and pseudopolyposis by contrast barium enema examination. Diffuse inflammation is detected in the intestinal mucosa by digestive tract endoscopy. Furthermore, histopathological features are destruction of mucosal epithelia, ulcer, reduction of embryonic cells, and diffuse infiltration of inflammatory cells into the lamina propria. The symptoms are abdominal pain, diarrhea, bloody stool, easy bleeding, and such. Most patients follow a chronic course of recrudescence and remission, and therefore long-term continuous treatment and monitoring is required.

In Japan, the number of patients with ulcerative colitis has rapidly increased since 1975 when it was designated as an intractable disease. According to the 2004 Public Health Services Administration Report by the Ministry of Health, Labor and Welfare of the Japanese government, the number of people with a Medical Care Certificate for Specified Diseases is estimated to be 79,897, and is increasing every year. No gender difference is seen in the onset of ulcerative colitis. Ulcerative colitis can occur at any age, but most frequently it occurs among people in their 20 s. Since it occurs frequently in young people and takes a chronic course, patients have difficulties in maintaining a stable social life, such as attending school and going to work, and thus their QOL is significantly compromised.

Ulcerative colitis is a cryptogenic disease. Although the number of patients is increasing every year, no radical therapeutic method has been found. Currently, 5-ASA preparations (salazosulfapyridine, mesalazine, and the like), adrenal medullary steroids (prednisolone, betamethasone, and the like), and immunosuppressants (azathioprine, 6-MP, and the like) are used in the treatment; however, ulcerative colitis is often recurrent in spite of medication. Surgical therapy has been regarded as the ultimate form of treatment for patients who are resistant to strong antiinflammatory agents or immunosuppressants. Furthermore, leukocytapheresis (LCAP) has been developed as a therapeutic method for steroid-resistant ulcerative colitis patients; however, there are great differences in the therapeutic effect among individuals. In addition, recent reports describe new therapeutic methods such as epidermal growth factor (EGF) enema (Non-patent Document 1), novel compounds that suppress the adhesion of inflammatory cells (Patent Document 1), methods for suppressing hyperimmune response (Patent Document 2), and the like. However, there is no radical therapeutic agent. Thus, new agents to improve therapeutic effects, such as agents for improving symptoms, preventing recurrence, and improving QOL are desirable.

Proteoglycans can be found in the extracellular matrix of various tissues. A proteoglycan is constructed by linking a glycosaminoglycan (GAG) chain to a core protein. It modulates interactions with cell surface receptors or other extracellular matrices (Non-patent Document 2). Six types of proteoglycans are known: heparan sulfate (HS), chondroitin sulfate (CS), heparin, dermatan sulfate (DC), keratan sulfate (KS), and hyaluronic acid (Non-patent Document 3).

It is already known that chondroitin sulfate proteoglycan (CSPG) produced by damaged nerve cells inhibits axon regeneration (Non-patent Document 4) and that the use of chondroitinase, which is known as an enzyme with CS-degrading activity, can enhance axon regeneration (Patent Documents 3 and 4).

Versican (also known as PG-M), which is one of the chondroitin sulfate proteoglycans, comprises a hyaluronic acid-binding domain near the N terminus, a glycosaminoglycan-attaching domain in the middle, and an EGF-like domain, C-type lectin-like domain and complement regulatory protein-like domain at the C terminus (Non-patent Document 5). The human versican gene consists of 15 exons. As a result of selective splicing, versican binds to hyaluronic acid with high affinity via its hyaluronic acid-binding domain, and to sulfated glycolipids and extracellular matrix components, tenascin-R and fibrin-1, via its C-type lectin-like domain. Furthermore, versican binds to an EGF receptor via its EGF-like domain and at least partly enhances cell growth. Versican is also known to have an activity of inhibiting cell adhesion via its chondroitin sulfate chain (Non-patent Document 6). There is also a report describing that overexpression of versican suppresses the migration of neural crest cells in congenital spina bifida mice (Non-patent Document 7); however, there is no known therapeutic method based on controlling the expression of versican. Detecting or identifying colon cell proliferative diseases by identifying methylated CpG dinucleotides and non-methylated CpG dinucleotides in the versican gene is known (Patent Document 5). However, there is no report on the correlation of versican with ulcerative colitis.

[Patent Document 1] U.S. Pat. No. 6,943,180
[Patent Document 2] U.S. Pat. No. 6,764,838

### [Patent Document 3] WO 2003/074080

### [Patent Document 4] WO 2003/015612

### [Patent Document 5] WO 2003/072820

### [Non-patent Document 1] Sinha, A., N Engl J Med. (2003) 24, 349(4): 350-7

### [Non-patent Document 2] Corvetti, L., J Neurosci. (2005) 25(31): 7150-7158

### [Non-patent Document 3] Lozzo, R. V., FASEB J. (1996) 10: 598-614

### [Non-patent Document 4] Smith-Thomas, J Cell Sci. (1994) 107: 1687-1695

### [Non-patent Document 5] Kiani, C., Cell Res. (2002) 12: 19-32

### [Non-patent Document 6] Sheng, W., Mol Biol Cell. (2005) 16: 1330-40

### [Non-patent Document 7] Henderson, D. J., Mech Dev. (1997) 69(1-2): 39-51

### DISCLOSURE OF THE INVENTION

### Problems to Be Solved by the Invention

An objective of the present invention is to provide intestinal inflammation-suppressing agents, therapeutic agents against inflammatory bowel diseases that comprise the above agents as active ingredients, and methods of screening for intestinal inflammation-suppressing agents.

### Means to Solve the Problems

An objective of the present invention is to provide agents capable of suppressing the accumulation of chondroitin sulfate proteoglycans (CSPG) in the intestine, which are useful for treating or preventing inflammatory bowel diseases (IBD) caused by the accumulation of chondroitin sulfate proteoglycans (CSPG).

The present inventors conducted dedicated studies to develop such agents, and conceived that excess accumulation of chondroitin sulfate proteoglycans (CSPG) might enhance intestinal inflammation, although it was not considered to be a cause of inflammatory bowel diseases.

The present inventors carried out research based on this assumption and as a result, discovered that suppressing the expression of versican, one of the chondroitin sulfate proteoglycans (CSPG), or administering ADAMTS-4 which has an activity of cleaving chondroitin sulfate proteoglycans, could suppress intestinal inflammation and thus inflammatory bowel diseases. Thus, the inventors completed the present invention. Substances that inhibit the production or accumulation of chondroitin sulfate proteoglycans are useful as intestinal inflammation-suppressing agents. In addition, the agents can also be used as therapeutic or preventive agents for inflammatory bowel diseases.

The present invention relates to intestinal inflammation-suppressing agents, therapeutic agents for inflammatory bowel diseases that comprise the above agents as an active ingredient, and to methods of screening for intestinal inflammation-suppressing agents. More specifically, the present invention provides:

[1] An intestinal inflammation-suppressing agent which comprises as an active ingredient a substance that inhibits the production or accumulation of a chondroitin sulfate proteoglycan.
[2] The agent of [1], wherein the substance has an activity of promoting the degradation of a chondroitin sulfate proteoglycan.
[3] The agent of [1], wherein the substance has an activity of inhibiting the synthesis of a chondroitin sulfate proteoglycan.
[4] The agent of [1], wherein the substance has an activity of desulfating a chondroitin sulfate proteoglycan.
[5] The agent of [1], wherein the substance has an activity of inhibiting the sulfation of a chondroitin sulfate proteoglycan.
[6] The agent of any of [1] to [5], wherein the production or accumulation of a chondroitin sulfate proteoglycan is inhibited in the large intestine or the small intestine.
[7] The agent of any of [1] to [6], which is used for treating or preventing an inflammatory bowel disease.
[8] The agent of [7], wherein the inflammatory bowel disease is ulcerative colitis.
[9] The agent of [7], wherein the inflammatory bowel disease is Crohn's disease.
[10] A method of screening for an intestinal inflammation-suppressing agent, which comprises selecting from a test sample a substance with an activity of inhibiting the production or accumulation of a chondroitin sulfate proteoglycan.
[11] The method of [10], which comprises the step of selecting a substance with the activity of any of:
(a) promoting the degradation of a chondroitin sulfate proteoglycan;
(b) inhibiting the synthesis of a chondroitin sulfate proteoglycan;
(c) desulfating a chondroitin sulfate proteoglycan; and
(d) inhibiting the sulfation of a chondroitin sulfate proteoglycan.
[12] The method of [10] or [11], wherein the intestinal inflammation-suppressing agent is used for treating or preventing an inflammatory bowel disease.

Further, the present invention relates to the followings:

[13] Use of the agent of any of [1] to [9] for producing an intestinal inflammation-suppressing agent.
[14] A method of treating an inflammatory bowel disease which comprises the step of administering the agent of any of [1] to [9] to a subject (such as patients).
[15] A composition which comprises the agent of any of [1] to [9] and a pharmaceutically acceptable carrier.

### EFFECT OF THE INVENTION

The present invention has demonstrated that the production and accumulation of chondroitin sulfate proteoglycans is involved in the development of intestinal inflammation. Inhibiting the production and accumulation of chondroitin sulfate proteoglycans was shown to suppress the onset of intestinal inflammation. Thus, therapeutic agents for intestinal inflammation can be provided based on this new concept. In particular, patient number of ulcerative colitis, which is one of inflammatory bowel diseases, is increasing year by year. Therefore, such therapeutic agents based on this new concept have great medical and industrial significance.

### BRIEF DESCRIPTION OF THE DRAWINGS

FIG. 1 is a graph showing the therapeutic effect of versican siRNA in ulcerative colitis model mice. The horizontal axis indicates the number of days, and the vertical axis indicates the disease activity index (DAI). *, p<0.05; **, p<0.01 (t test)

FIG. 2 contains a graph and photographs showing the therapeutic effect of versican siRNA in ulcerative colitis model mice. The photographs show large intestines from the control group and the versican siRNA-treated group. The graph shows the length of the intestines measured. *, p<0.05; **, p<0.01 (t test)

FIG. 3 contains photographs showing the effect of versican siRNA administration in suppressing the expression of versican in ulcerative colitis model mice. The photographs show electrophoretic patterns of the PCR amplification products of versican mRNA.

FIG. 4-1 contains photographs of histological images showing the therapeutic effect of versican siRNA in ulcerative colitis model mice. Macrophages (F4/80 positive cells) were stained.

FIG. 4-2 is a continuation of the photographs in FIG. 4-1. Reticular fibers/fibroblasts (ER-TR7 positive cells) were stained.

FIG. 4-3 is a continuation of the photographs in FIG. 4-2. Chondroitin sulfate proteoglycans were stained.

FIG. 5 contains photographs showing suppression of the CSPG deposition by administering a functional ADAMTS-4 peptide. The photographs show immunohistological images of large intestines on day 8 of DSS colitis. CS56 (CSPG) was stained brown. The upper, 100 fold; bottom, 400 fold.

FIG. 6-1 contains photographs showing suppression of the infiltration of macrophages and fibroblasts by administering a functional ADAMTS-4 peptide. F4/80 (the upper, 100 fold; bottom, 400 fold) was stained brown. The images demonstrate that administration of the functional ADAMTS-4 peptide not only suppressed cellular infiltration, but also conserved the tissue structure remarkably well.

FIG. 6-2 is a continuation of the photographs in FIG. 6-1. ER-TR7 (100 fold) was stained brown. The images demonstrate that administration of the functional ADAMTS-4 peptide not only suppressed cellular infiltration, but also conserved the tissue structure remarkably well.

### BEST MODE FOR CARRYING OUT THE INVENTION

The present invention is specifically described below:

Pathological conditions associated with ulcerative colitis, a representative inflammatory bowel disease, include inflammation of large intestinal mucosa. The present inventors focused on the functions of chondroitin sulfate proteoglycans in order to establish that the improvement of inflammatory conditions in large intestinal mucosa is an effective therapeutic method for ulcerative colitis. The inventors then suppressed the accumulation of chondroitin sulfate proteoglycans in a mouse model for ulcerative colitis, and closely analyzed this condition to reveal that the accumulation of chondroitin sulfate proteoglycans was ameliorated in many cells, and inflammatory conditions, such as reduction of inflammatory activity and atrophy suppression, were also improved as compared with those in the large intestinal mucosa of wild-type mice. Specifically, the inventors discovered that inhibiting the production or accumulation of chondroitin sulfate proteoglycans facilitated the amelioration of abnormal accumulations of chondroitin sulfate proteoglycans in large intestinal mucosa, a factor deeply involved in ulcerative colitis, and thus leading to the improvement of intestinal inflammation.

The present invention relates to intestinal inflammation-suppressing agents, which comprise substances that inhibit the production or accumulation of chondroitin sulfate proteoglycans as active ingredients.

In the present invention, “chondroitin sulfate proteoglycans” are a type of proteoglycan and collectively refer to compounds in which proteins (core proteins) are covalently linked to chondroitin sulfate/dermatan sulfate, which are representative sulfated mucopolysaccharides.

In the present invention, preferable “chondroitin sulfate proteoglycans” are human chondroitin sulfate proteoglycans. The species from which the proteoglycans are derived are not particularly limited. Proteins of nonhuman organisms (homologues, orthologues, and such) that are equivalent to the chondroitin sulfate proteoglycans are also included in the chondroitin sulfate proteoglycans of the present invention. For example, the present invention can be conducted as long as the species has a protein corresponding to a human chondroitin sulfate proteoglycan and equivalent to a human chondroitin sulfate proteoglycan. Furthermore, the chondroitin sulfate proteoglycans in the present invention also include so-called part-time proteoglycans, which are temporarily linked with glycosaminoglycan (GAG) chains to become proteoglycans upon inflammation or the like.

Examples of the chondroitin sulfate proteoglycans described below are aggrecan, versican, neurocan, brevican, β-glycan, decorin, biglycan, fibromodulin, and PG-Lb. However, the chondroitin sulfate proteoglycans in the present invention are not limited to these examples, and may be any substances with a chondroitin sulfate proteoglycan activity. Herein, chondroitin sulfate proteoglycan activities include, for example, cell adhesion ability and cell growth promotion. Those skilled in the art can assay chondroitin sulfate proteoglycan activities by assaying cell division and growth of tumor cells (for example, Caco-2 and HT-29 cells) in the presence of a protein containing a partial amino acid sequence of a chondroitin sulfate proteoglycan, or a protein with high homology to such a partial amino acid (typically 70% homology or higher, preferably 80% or higher, more preferably 90% or higher, and most preferably 95% or higher). Proteins with the effect of promoting cell division and growth can be evaluated as proteins with a chondroitin sulfate proteoglycan activity (Int. J. Exp. Pathol. 2005 August, 86(4), 219-29; and Histochem Cell Biol. 2005 August, 124(2), 139-49). High homology means 50% or higher homology, preferably 70% or higher homology, more preferably 80% or higher homology, and still more preferably 90% or higher homology (for example, 95% or higher homology, or 96%, 97%, 98%, 99% or higher homology). Such homology can be determined using the mBLAST algorithm (Altschul et al., Proc. Natl. Acad. Sci. USA 1990, 87, 2264-8; Karlin and Altschul, Proc. Natl. Acad. Sci. USA 1993, 90, 5873-7).

In the present invention, “inflammation” refers to local responses to body tissue damages, and causes redness, swelling, fever, and such in general. Inflammation includes, for example, inflammations accompanied by erosion, ulcer, and such in the large intestinal mucosa, but is not limited thereto.

Herein, “inhibition of production or accumulation” of chondroitin sulfate proteoglycans includes, for example, “promotion of degradation”, “inhibition of synthesis”, “desulfation”, and “inhibition of sulfation” of chondroitin sulfate proteoglycans; however, “inhibition of production or accumulation” is not limited thereto, and it refers to a reduction or loss of the amount, function or activity of a chondroitin sulfate proteoglycan, as compared to a comparison subject. Herein, “substances that inhibit the production or accumulation” of chondroitin sulfate proteoglycans are not particularly limited. Such substances are preferably “substances with the effect of promoting the degradation of chondroitin sulfate proteoglycans”, “substances with the effect of inhibiting the synthesis of chondroitin sulfate proteoglycans”, “substances with the effect of desulfating chondroitin sulfate proteoglycans”, or “substances with the effect of inhibiting the sulfation of chondroitin sulfate proteoglycans”.

“Promotion of degradation” of chondroitin sulfate proteoglycans includes, for example, inhibition of the expression of core proteins of chondroitin sulfate proteoglycans, and a reduction in the abundance of the core proteins. Herein, “core proteins of chondroitin sulfate proteoglycans” include, for example, aggrecan, versican, neurocan, and brevican for matrix-type chondroitin sulfate proteoglycans; and β-glycan, decorin, biglycan, fibromodulin, and PG-Lb for membrane chondroitin sulfate proteoglycans. Those described above are all examples, but the core proteins are not limited to these and a wide variety of proteins may serve as chondroitin sulfate proteoglycan cores.

“Expression” includes “transcription” from genes, “translation” into polypeptides, and “suppression of degradation” of proteins. The “expression of core proteins of chondroitin sulfate proteoglycans” refers to transcription and translation of the genes encoding core proteins of chondroitin sulfate proteoglycans, or production of core proteins of chondroitin sulfate proteoglycans through transcription and translation. Furthermore, the “function of core proteins of chondroitin sulfate proteoglycans” includes, for example, their function in binding to chondroitin sulfate, and in binding with other cellular components. Those skilled in the art can appropriately evaluate (measure) the various above-mentioned functions using general methods. Specifically, the evaluation can be performed using the methods described in the Examples herein below, or using the same methods with appropriate modifications.

The “promotion of degradation” of chondroitin sulfate proteoglycans may also be an increase in the expression of enzymes that cleave or degrade chondroitin sulfate proteoglycans, or of enzymes involved in the cleavage or degradation of proteoglycans. Such enzymes include, for example, metalloproteinases (for example, ADAMTS-1, ADAMTS-4, and ADAMTS-5), chondroitinase, and calpain I, but are not limited thereto. The “promotion of degradation” may be a reduction in the abundance of chondroitin sulfate proteoglycans caused by administering all or some of the enzymes.

Alternatively, “promotion of degradation” may be achieved by administering a substance that promotes the suppression of expression of chondroitin sulfate proteoglycans. Such substances include, for example, n-butylate, diethylcarbamazepine, tunicamycin, non-steroidal estrogen, and cyclofenil diphenol, but are not limited thereto.

Preferred embodiments of the “substance with the activity of promoting degradation” include, for example, compounds (nucleic acids) selected from the group consisting of:

(a) antisense nucleic acids against transcripts of the genes encoding the core proteins of chondroitin sulfate proteoglycans, or portions thereof;
(b) nucleic acids with the ribozyme activity of specifically cleaving transcripts of genes encoding core proteins of chondroitin sulfate proteoglycans; and
(c) nucleic acids with the activity of using RNAi effect to inhibit the expression of genes encoding core proteins of chondroitin sulfate proteoglycans.

Furthermore, the “substances with the activity of promoting degradation” include, for example, compounds selected from the group consisting of:

(a) antibodies that bind to core proteins of chondroitin sulfate proteoglycans:
(b) chondroitin sulfate proteoglycan variants that are dominant-negative for core proteins of chondroitin sulfate proteoglycans; and
(c) low-molecular-weight compounds that bind to core proteins of chondroitin sulfate proteoglycans.

“Inhibition of synthesis” of chondroitin sulfate proteoglycans includes, for example, inhibition of biosynthesis of glycosaminoglycans and inhibition of enzymes involved in the synthesis of chondroitin sulfate proteoglycans, but is not limited thereto. The inhibition refers to inhibition of any of the processes of chondroitin sulfate proteoglycan synthesis.

As substances that inhibit the synthesis of chondroitin sulfate proteoglycans, substances inhibiting the biosynthesis of glycosaminoglycans include, for example, β-D-xyloside, 2-deoxy-D-glucose (2-DG), ethane-1-hydroxy-1,1-diphosphonate (ETDP), and 5-hexyl-2-deoxyuridine (HudR). Such substances inhibit the biosynthesis of glycosaminoglycans, and thereby inhibit the synthesis of chondroitin sulfate proteoglycans.

Meanwhile, enzymes involved in chondroitin synthesis include, for example, GalNAc4ST-1, GalNAc4ST-2, GALNAC4S-6ST, UA20ST, GalT-I, GalT-II, GlcAT-I, and XylosylT. The synthesis of chondroitin sulfate proteoglycans is inhibited by inhibiting such enzymes, suppressing the expression thereof, or the like.

Preferred embodiments of “substances with the activity of inhibiting synthesis” include, for example, compounds (nucleic acids) selected from the group consisting of:

(a) antisense nucleic acids against transcripts of genes encoding chondroitin sulfate proteoglycan synthetases, or portions thereof;
(b) nucleic acids with the ribozyme activity of specifically cleaving transcripts of the genes encoding chondroitin sulfate proteoglycan synthetases; and
(c) nucleic acids with the activity of using RNAi effect to inhibit the expression of genes encoding chondroitin sulfate proteoglycan synthetases.

The “substances with the activity of inhibiting synthesis” also include, for example, compounds selected from the group consisting of:

(a) antibodies that bind to chondroitin sulfate proteoglycan synthetases;
(b) chondroitin sulfate proteoglycan synthetase variants that are dominant-negative for chondroitin sulfate proteoglycan synthetases; and
(c) low-molecular-weight compounds that bind to chondroitin sulfate proteoglycan synthetases.

The “desulfation” of chondroitin sulfate proteoglycans refers to the removal of a sulfate group from chondroitin sulfate proteoglycans, and includes, for example, desulfation by an endogenous or exogenously-administered desulfation enzyme, and suppression of sulfation by a sulfation-suppressing compound, but is not limited thereto. “Desulfation” refers to the process of sulfate group removal.

Such desulfation enzymes include, for example, chondroitin-4-sulfatase and chondroitin-6-sulfatase. Sulfation-suppressing compounds include, for example, chlorate and EGF receptor antagonists.

Preferred embodiments of such “substances with desulfating activity” include, for example, compounds (nucleic acids) selected from the group consisting of:

(a) antisense nucleic acids against transcripts of genes encoding proteins that suppress chondroitin sulfate proteoglycan-desulfating enzymes, or portions thereof;
(b) nucleic acids with the ribozyme activity of specifically cleaving transcripts of the genes encoding proteins that suppress chondroitin sulfate proteoglycan-desulfating enzymes; and
(c) nucleic acids with the activity of using RNAi effect to inhibit the expression of genes encoding proteins that suppress chondroitin sulfate proteoglycan-desulfating enzymes.

The “substances with desulfating activity” also include, for example, compounds selected from the group consisting of:

(a) antibodies that bind to compounds that suppress chondroitin sulfate proteoglycan-desulfating enzymes;
(b) variants of proteins that suppress chondroitin sulfate proteoglycan-desulfating enzymes, which are dominant-negative for proteins that suppress chondroitin sulfate proteoglycan-desulfating enzymes; and
(c) low-molecular-weight compounds that bind to compounds that suppress chondroitin sulfate proteoglycan-desulfating enzymes.

Herein, “desulfation-suppressing compounds” include not only proteins but also non-pertinacious compounds such as coenzymes.

The “activity of inhibiting sulfation” of chondroitin sulfate proteoglycans includes, for example, inhibition of sulfotransferases, but is not limited thereto. The activity refers to the inhibition of sulfation in the process of chondroitin sulfate proteoglycan synthesis.

Such sulfotransferases include, for example, chondroitin D-N-acetylgalactosamine-4-O-sulfotransferase 1 (C4ST-1), chondroitin D-N-acetylgalactosamine-4-O-sulfotransferase 2 (C4ST-2), chondroitin D-N-acetylgalactosamine-4-O-sulfotransferase 3 (C4ST-3), D4ST, C6ST-1, and C6ST-2.

Preferred embodiments of “substances with the activity of inhibiting sulfation” include, for example, compounds (nucleic acids) selected from the group consisting of:

(a) antisense nucleic acids against transcripts of genes encoding sulfotransferases for chondroitin sulfate proteoglycans, or portions thereof;
(b) nucleic acids with the ribozyme activity of specifically cleaving transcripts of the genes encoding sulfotransferases for chondroitin sulfate proteoglycans; and
(c) nucleic acids with the activity of using RNAi effect to inhibit the expression of genes encoding sulfotransferases for chondroitin sulfate proteoglycans.

The “substances with the activity of inhibiting sulfation” also include, for example, compounds selected from the group consisting of:

(a) antibodies that bind to sulfotransferases for chondroitin sulfate proteoglycans;
(b) sulfotransferase variants for chondroitin sulfate proteoglycans; and
(c) low-molecular-weight compounds that bind to sulfotransferases for chondroitin sulfate proteoglycans.

The above enzymes shown as examples include not only single enzymes that correspond to single genes, but also groups of enzymes that share certain characteristics. For example, chondroitinase is a collective name for enzymes such as ABC, AC, and B, whose substrate specificities or such are different, but which share the characteristics of mucopolysaccharide-degrading enzymes. For example, chondroitinase AC I eliminatively cleaves the N-acetylhexosaminide linkages of chondroitin sulfates (A, C, and E), chondroitin, chondroitin sulfate-dermatan sulfate hybrids, and hyaluronic acid, and yields oligosaccharides with Δ4-glucuronate residues at the non-reducing ends. This enzyme does not act on dermatan sulfate (chondroitin sulfate B, which has L-iduronic acid for a hexuronic acid), keratan sulfate, heparan sulfate, and heparin. Meanwhile, chondroitinase AC II eliminatively cleaves the N-acetylhexosaminide linkages of chondroitin, chondroitin sulfate A, and chondroitin sulfate C, and yields Δ4-unsaturated disaccharides (ΔDi-0S, ΔDi-4S, and ΔDi-6S). This enzyme also acts well on hyaluronic acid. The enzyme does not act on dermatan sulfate (chondroitin sulfate B), which is therefore a competitive inhibitor of the enzyme. Chondroitinase B (dermatanase) eliminatively cleaves N-acetylhexosaminide linkages to L-iduronic acids in dermatan sulfate, and yields oligosaccharides (di- and tetra-saccharides) with Δ4-hexuronate residues at the non-reducing ends. This enzyme acts on neither chondroitin sulfate A nor chondroitin sulfate C, which are free of L-iduronic acid. Dermatan, which is a derivative of dermatan sulfate in which the sulfate group is removed, does not serve as a substrate for this enzyme. This enzyme preferentially cleaves portions of dermatan sulfate in which the second of the L-iduronic acid units are sulfated. Chondroitinase ABC eliminatively cleaves N-acetylhexosaminide linkages of chondroitin sulfate A, chondroitin sulfate C, dermatan sulfate, chondroitin, and hyaluronic acid, and yields mainly disaccharides with Δ4-hexuronate groups at the non-reducing ends. This enzyme does not act on keratan sulfate, heparin, and heparan sulfate. Chondroitinases collectively refer to enzymes sharing the characteristics of mucopolysaccharide-degrading enzymes while also having different characteristics as described above, and they are not limited to chondroitinase ACI, chondroitinase AC II, chondroitinase B, and chondroitinase ABC as exemplified above.

Further, on a genomic DNA level, such groups of enzymes sharing features do not necessarily correspond to single genes. For example, both chondroitin-4-sulfatase and chondroitin-6-sulfatase can be retrieved from the public gene database Genbank as sequences referred to by multiple accession numbers (for example, Genbank accession Nos: NT_{—}039500 (a portion thereof is shown under accession No: CAAA01098429 (SEQ ID NO: 74)), NT_{—}078575, NT_{—}039353, NW_{—}001030904, NW_{—}001030811, NW_{—}001030796, and NW_{—}000349).

The above example proteins that correspond to single genes are shown below: Specifically, below are the accession numbers in the public gene database Genbank, nucleotide sequences, and amino acid sequences for human genes encoding:

aggrecan, versican, neurocan, brevican, β-glycan, decorin, biglycan, fibromodulin, and PG-Lb, which are shown above as examples of chondroitin sulfate proteoglycans; ADAMTS-1, ADAMTS-4, ADAMTS-5, and calpain I, which are shown above as examples of enzymes that cleave or degrade chondroitin sulfate proteoglycans or related enzymes; GalNAc4ST-1, GalNAc4ST-2, GALNAC4S-6ST, UA20ST. GalT-I, GalT-II, GlcAT-I, and XylosylT, which are shown above as examples of enzymes involved in chondroitin synthesis; C4ST-1, C4ST-2, C4ST-3, D4ST, C6ST-1, and C6ST-2, which are shown above as examples of sulfotransferases.
Aggrecan (Accession No: NM_{—}007424; nucleotide sequence: SEQ ID NO: 1; amino acid sequence: SEQ ID NO: 2)
Versican (Accession No: BC096495; nucleotide sequence: SEQ ID NO: 3; amino acid sequence: SEQ ID NO: 4)
Neurocan (Accession No: NM_{—}010875; nucleotide sequence: SEQ ID NO: 5; amino acid sequence: SEQ ID NO: 6)
Brevican (Accession No: NM_{—}007529; nucleotide sequence: SEQ ID NO: 7; amino acid sequence: SEQ ID NO: 8)
β-glycan (Accession No: AF039601; nucleotide sequence: SEQ ID NO: 9; amino acid sequence: SEQ ID NO: 10)
Decorin (Accession No: NM_{—}007833; nucleotide sequence: SEQ ID NO: 11; amino acid sequence: SEQ ID NO: 12)
Biglycan (Accession No: BC057185; nucleotide sequence: SEQ ID NO: 13; amino acid sequence: SEQ ID NO: 14)
Fibromodulin (Accession No: NM_{—}021355; nucleotide sequence: SEQ ID NO: 15; amino acid sequence: SEQ ID NO: 16)
PG-Lb (Accession No: NM_{—}007884; nucleotide sequence: SEQ ID NO: 17; amino acid sequence: SEQ ID NO: 18)
ADAMTS-1 (Accession No: NM_{—}009621; nucleotide sequence: SEQ ID NO: 19; amino acid sequence: SEQ ID NO: 20)
ADAMTS-4 (Accession No: NM_{—}172845; nucleotide sequence: SEQ ID NO: 21; amino acid sequence: SEQ ID NO: 22)
ADAMTS-5 (Accession No: AF140673; nucleotide sequence: SEQ ID NO: 23; amino acid sequence: SEQ ID NO: 24)
Calpain I (Accession No: NM_{—}007600; nucleotide sequence: SEQ ID NO: 25; amino acid sequence: SEQ ID NO: 26)
GalNAc4ST-1 (Accession No: NM_{—}175140; nucleotide sequence: SEQ ID NO: 27; amino acid sequence: SEQ ID NO: 28)
GalNAc4ST-2 (Accession No: NM_{—}199055; nucleotide sequence: SEQ ID NO: 29; amino acid sequence: SEQ ID NO: 30)
GALNAC4S-6ST (Accession No: NM_{—}029935; nucleotide sequence: SEQ ID NO: 31; amino acid sequence: SEQ ID NO: 32)
UA20ST (Accession No: NM_{—}177387; nucleotide sequence: SEQ ID NO: 33; amino acid sequence: SEQ ID NO: 34)
GalT-I (Accession No: NM_{—}016769; nucleotide sequence: SEQ ID NO: 35; amino acid sequence: SEQ ID NO: 36)
GalT-II (Accession No: BC064767; nucleotide sequence: SEQ ID NO: 37; amino acid sequence: SEQ ID NO: 38)
GlcAT-I (Accession No: BC058082; nucleotide sequence: SEQ ID NO: 39; amino acid sequence: SEQ ID NO: 40), or Accession No: NM_{—}024256; nucleotide sequence: SEQ ID NO: 41: amino acid sequence: SEQ ID NO: 42)
XylosylT (Accession No: NM_{—}145828; nucleotide sequence: SEQ ID NO: 43; amino acid sequence: SEQ ID NO: 44)
C4ST-1 (Accession No: NM_{—}021439; nucleotide sequence: SEQ ID NO: 45; amino acid sequence: SEQ ID NO: 46)
C4ST-2 (Accession No: NM_{—}021528; nucleotide sequence: SEQ ID NO: 47; amino acid sequence: SEQ ID NO: 48)
C4ST-3 (Accession No: XM_{—}355798; nucleotide sequence: SEQ ID NO: 49; amino acid sequence: SEQ ID NO: 50)
D4ST (Accession No: NM_{—}028117; nucleotide sequence: SEQ ID NO: 51; amino acid sequence: SEQ ID NO: 52)
C6ST-1 (Accession No: NM_{—}016803; nucleotide sequence: SEQ ID NO: 53; amino acid sequence: SEQ ID NO: 54)
C6ST-2 (Accession No: AB046929; nucleotide sequence: SEQ ID NO: 55; amino acid sequence: SEQ ID NO: 56)

In addition to the proteins listed above, the proteins of the present invention include those exhibiting high homology (typically 70% or higher, preferably 80% or higher, more preferably 90% or higher, and most preferably 95% or higher) to sequences shown in the Sequence Listing and with a function of the proteins listed above (for example, the function of binding to intracellular components). The proteins listed above are, for example, proteins comprising an amino acid sequence with an addition, deletion, substitution, or insertion of one or more amino acids in any of the amino acid sequences of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, and 56, in which the number of altered amino acids is typically 30 amino acids or less, preferably ten amino acids or less, more preferably five amino acids or less, and most preferably three amino acids or less.

The above-described genes of the present invention include, for example, endogenous genes of other organisms which correspond to DNAs comprising any of the nucleotide sequences of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, and 55 (homologues to the human genes described above, or the like).

Each of the endogenous DNAs of other organisms which correspond to DNAs comprising any of the nucleotide sequences of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, and 55 are generally highly homologous to a DNA of any of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, and 55. High homology means 50% or higher homology, preferably 70% or higher homology, more preferably 80% or higher homology, and still more preferably 90% or higher homology (for example, 95% or higher, or 96%, 97%, 98%, or 99% or higher). Homology can be determined using the mBLAST algorithm (Altschul et al., Proc. Natl. Acad. Sci. USA 1990, 87, 2264-8; Karlin and Altschul, Proc. Natl. Acad. Sci. USA 1993, 90, 5873-7). When the DNAs have been isolated from the body, each of them may hybridize under stringent conditions to a DNA of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, or 55. Herein, stringent conditions include, for example, “2×SSC, 0.1% SDS, 50° C.”, “2×SSC, 0.1% SDS, 42° C.”, and “1×SSC, 0.1% SDS, 37° C.”; more stringent conditions include “2×SSC, 0.1% SDS, 65° C.”, “0.5×SSC, 0.1% SDS, 42° C.”, and “0.2×SSC, 0.1% SDS, 65° C.”.

Those skilled in the art can appropriately obtain proteins functionally equivalent to the above-described proteins from the above-described highly homologous proteins by using methods for assaying the activity of promoting the degradation of CSPGs, inhibiting the synthesis of CSPGs, desulfating CSPGs, or inhibiting the sulfation of CSPGs. Specific methods for assaying the activities are described below in a section on the screening methods of the present invention. Further, based on the nucleotide sequences of the above-described genes, those skilled in the art can appropriately obtain endogenous genes of other organisms that correspond to the above-described genes. In the present invention, the above-described proteins and genes in non-human organisms, which correspond to the above-described proteins and genes, or the above-described proteins and genes that are functionally equivalent to the above-described proteins and genes, may simply be referred to using the above-described names.

The proteins of the present invention can be prepared not only as natural proteins but also as recombinant proteins using genetic recombination techniques. The natural proteins can be prepared by, for example, methods of subjecting cell extracts (tissue extracts) that may express the above-described proteins to affinity chromatography using antibodies against the above-described proteins. On the other hand, the recombinant proteins can be prepared, for example, by culturing cells transformed with DNAs encoding the proteins described above. The above-described proteins of the present invention can be suitably used, for example, in the screening methods described herein below.

In the present invention, “nucleic acids” refer to both RNAs and DNAs. Chemically synthesized nucleic acid analogs, such as so-called “PNAs” (peptide nucleic acids), are also included in the nucleic acids of the present invention. PNAs are nucleic acids in which the fundamental backbone structure of nucleic acids, the pentose-phosphate backbone, is replaced by a polyamide backbone with glycine units. PNAs have a three-dimensional structure quite similar to that of nucleic acids.

Methods for inhibiting the expression of specific endogenous genes using antisense technology are well known to those skilled in the art. There are a number of causes for the action of antisense nucleic acids in inhibiting target gene expression, including: inhibition of transcription initiation by triplex formation;

transcription inhibition by hybrid formation at a site with a local open loop structure generated by an RNA polymerase;
transcription inhibition by hybrid formation with the RNA being synthesized;
splicing inhibition by hybrid formation at an intron-exon junction;
splicing inhibition by hybrid formation at the site of spliceosome formation:
inhibition of transport from the nucleus to the cytoplasm by hybrid formation with mRNA;
splicing inhibition by hybrid formation at the capping site or poly(A) addition site;
inhibition of translation initiation by hybrid formation at the translation initiation factor binding site:
inhibition of translation by hybrid formation at the ribosome binding site adjacent to the start codon;
inhibition of peptide chain elongation by hybrid formation in the translational region of mRNA or at the polysome binding site of mRNA; and
inhibition of gene expression by hybrid formation at the protein-nucleic acid interaction sites. Thus, antisense nucleic acids inhibit the expression of target genes by inhibiting various processes, such as transcription, splicing, and translation (Hirashima and Inoue, Shin Seikagaku Jikken Koza 2 (New Courses in Experimental Biochemistry 2), Kakusan (Nucleic Acids) IV: “Idenshi no Fukusei to Hatsugen (Gene replication and expression)”, Ed. The Japanese Biochemical Society, Tokyo Kagakudojin, 1993, pp. 319-347).

The antisense nucleic acids used in the present invention may inhibit the expression and/or function of genes encoding any of the CSPG core proteins, synthetases, proteins suppressing desulfation enzymes, and sulfotransferases described above, based on any of the actions described above. In one embodiment, antisense sequences designed to be complementary to an untranslated region adjacent to the 5′ end of an mRNA for a gene encoding an above-described CSPG core protein, synthetase, desulfation enzyme-suppressing protein, or sulfotransferase may be effective for inhibiting translation of the gene. Sequences complementary to a coding region or 3′-untranslated region can also be used. Thus, the antisense nucleic acids to be used in the present invention include not only nucleic acids comprising sequences antisense to the coding regions, but also nucleic acids comprising sequences antisense to untranslated regions of genes encoding the above-described CSPG core proteins, synthetases, desulfation enzyme-suppressing proteins, or sulfotransferases. Such antisense nucleic acids to be used are linked downstream of adequate promoters and are preferably linked with transcription termination signals on the 3′ side. Nucleic acids thus prepared can be introduced into desired animals (cells) using known methods. The sequences of the antisense nucleic acids are preferably complementary to a gene or portion thereof encoding a CSPG core protein, synthetase, desulfation enzyme-suppressing protein, or sulfotransferase that is endogenous to the animals (cells) to be transformed with them. However, the sequences need not be perfectly complementary, as long as the antisense nucleic acids can effectively suppress expression of a gene. The transcribed RNAs preferably have 90% or higher, and most preferably 95% or higher complementarity to target gene transcripts. To effectively inhibit target gene expression using antisense nucleic acids, the antisense nucleic acids are preferably at least 15 nucleotides long, and less than 25 nucleotides long. However, the lengths of the antisense nucleic acids of the present invention are not limited to the lengths mentioned above, and they may be 100 nucleotides or more, or 500 nucleotides or more.

The antisense nucleic acids of the preset invention are not particularly limited, and can be prepared, for example, based on the nucleotide sequence of a versican gene (GenBank Accession No: BC096495; SEQ ID NO: 3), C4ST-1 (GenBank Accession No: NM_{—}021439; SEQ ID NO: 45), C4ST-2 (GenBank Accession NO: NM_{—}021528; SEQ ID NO: 47), C4ST-3 (GenBank Accession NO: XM_{—}355798; SEQ ID NO: 49), or such.

Expression of the above-mentioned genes encoding CSPG core proteins, synthetases, desulfation enzyme-suppressing proteins, or sulfotransferases can also be inhibited using ribozymes or ribozyme-encoding DNAs. Ribozymes refer to RNA molecules with catalytic activity. There are various ribozymes with different activities. Among others, studies that focused on ribozymes functioning as RNA-cleaving enzymes have enabled the design of ribozymes that cleave RNAs in a site-specific manner. Some ribozymes have 400 or more nucleotides, such as group I intron type ribozymes and M1 RNA, which is comprised by RNase P, but others, called hammerhead and hairpin ribozymes, have a catalytic domain of about 40 nucleotides (Koizumi, M. and Otsuka, E., Tanpakushitsu Kakusan Koso (Protein, Nucleic Acid, and Enzyme) 1990, 35, 2191).

For example, the autocatalytic domain of a hammerhead ribozyme cleaves the sequence G13U14C15 at the 3′ side of C15. Base pairing between U14 and A9 has been shown to be essential for this activity, and the sequence can be cleaved when C15 is substituted with A15 or U15 (Koizumi, M. et al., FEBS Lett. 1988, 228, 228). Restriction enzyme-like RNA-cleaving ribozymes that recognize the sequence UC, UU, or UA in target RNAs can be created by designing their substrate-binding sites to be complementary to an RNA sequence adjacent to a target site (Koizumi, M. et al., FEBS Lett. 1988, 239, 285; Koizumi, M. and Otsuka, E., Tanpakushitsu Kakusan Koso (Protein, Nucleic Acid, and Enzyme) 1990, 35, 2191; and Koizumi, M. et al., Nucl Acids Res. 1989, 17, 7059).

In addition, hairpin ribozymes are also useful for the purposes of the present invention. Such ribozymes are found in, for example, the minus strand of satellite RNAs of tobacco ringspot viruses (Buzayan, J. M., Nature 1986, 323, 349). It has been shown that target-specific RNA-cleaving ribozymes can also be created from hairpin ribozymes (Kikuchi, Y. and Sasaki, N., Nucl Acids Res. 1991, 19, 6751; and Kikuchi, Y Kagaku to Seibutsu (Chemistry and Biology) 1992, 30, 112). Thus, the expression of the above-described genes encoding CSPG core proteins, synthetases, desulfation enzyme-suppressing proteins, or sulfotransferases can be inhibited by using ribozymes to specifically cleave the gene transcripts.

The expression of endogenous genes can also be suppressed by RNA interference (hereinafter abbreviated as “RNAi”), using double-stranded RNAs comprising a sequence the same as or similar to a target gene sequence.

A great many disease-related genes have been rapidly identified since the entire human nucleotide sequence was revealed upon the recent completion of the genome project, and currently specific gene-targeted therapies and drugs are being actively developed. Of these, the application to gene therapy of small interfering RNAs (siRNAs), which produce the effect of specific post-transcriptional suppression, has been drawing attention. RNAi is a technology currently drawing attention in which double-stranded RNAs (dsRNAs) incorporated directly into cells suppress the expression of genes with sequences homologous to the dsRNAs. In mammalian cells, RNAi can be induced using short dsRNAs (siRNAs) and has many advantages: compared to knockout mice, RNAi has a stable effect, simple experiments, low costs, and so on.

RNA interference (RNAi) is a phenomenon where an mRNA comprising a nucleotide sequence complementary to a double-stranded RNA is degraded. RNAi is a method based on this phenomenon, in which the expression of an arbitrary gene is suppressed by artificially introducing a 21- to 23-mer double-stranded RNA (siRNA). In 1998, Fire et al. discovered using C. elegance that double-stranded RNA silences genes in a sequence-specific manner (Fire, A., Nature 1998, 391, 806-811). After elucidating the underlying mechanism of mRNA cleavage by 21- to 23-mer processed double-stranded RNA (Elbadhir, S. M., Nature 2001, 411, 494-498), identifying RNA-induced silencing complex (RISC) (Hammond, S. M., Nature 2000. 404, 293-296), and cloning Dicer (Bernstein, E., Nature 2001, 409, 363-366), Elbadhir et al. demonstrated in 2001 that siRNA could also suppress expression in a sequence-specific manner in mammalian cells (Zamore, P. O., Cell 2000, 101, 25-33). Thus, application of RNAi to gene therapy is highly expected.

Nucleic acids with inhibitory activity based on RNAi effect are generally referred to as siRNAs or shRNAs. RNAi is a phenomenon in which, when cells or such are introduced with short double-stranded RNAs (hereinafter abbreviated as “dsRNAs”) comprising sense RNAs that comprise sequences homologous to the mRNAs of a target gene, and antisense RNAs that comprise sequences homologous a sequence complementary thereto, the dsRNAs bind specifically and selectively to the target gene mRNAs, induce their disruption, and cleave the target gene, thereby effectively inhibiting (suppressing) target gene expression. For example, when dsRNAs are introduced into cells, the expression of genes with sequences homologous to the RNAs is suppressed (the genes are knocked down). As described above, RNAi can suppress the expression of target genes, and is thus drawing attention as a method applicable to gene therapy, or as a simple gene knockout method replacing conventional methods of gene disruption, which are based on complicated and inefficient homologous recombination. The RNAs to be used in RNAi are not necessarily perfectly identical to the genes or portions thereof that encode an above-described CSPG core protein, synthetase, desulfation enzyme-suppressing protein, or sulfotransferase; however, the RNAs are preferably perfectly homologous to the genes or portions thereof.

The targets of the siRNAs to be designed are not particularly limited, as long as they are genes encoding an above-described CSPG core protein, synthetase, desulfation enzyme-suppressing protein, or sulfotransferase. Any region of the gene can be a candidate for a target. For example, siRNAs may be prepared based on a nucleotide sequence of the versican gene (SEQ ID NO: 3), C4ST-1 gene (SEQ ID NO: 45), C4ST-2 gene (SEQ ID NO: 47), C4ST-3 gene (SEQ ID NO: 49), and such. More specifically, partial regions of such sequences may be used as candidates for the targets. For example, siRNAs may be prepared based on portions of the nucleotide sequences of a versican gene (SEQ ID NO: 57), C4ST-1 gene (SEQ ID NO: 58), C4ST-2 gene (SEQ ID NO: 59), C4ST-3 gene (SEQ ID NO: 60), C6ST-1 gene (SEQ ID NO: 61), C6ST-2 gene (SEQ ID NO: 62), GalNAc4ST-1 gene (SEQ ID NO: 63), GalNAc4ST-2 gene (SEQ ID NO: 64), GALNAC4S-6ST gene (SEQ ID NO: 65), or such. More specifically, examples of the siRNAs also include those targeted to the DNA sequences (SEQ ID NOs: 67 to 70) specifically shown herein.

The siRNAs can be introduced into cells by adopting methods of introducing cells with plasmid DNAs linked with siRNAs synthesized in vitro or methods that comprise annealing two RNA strands.

The two RNA molecules described above may be closed at one end or, for example, may be siRNAs with hairpin structures (shRNAs). shRNAs refer to short hairpin RNAs, which are RNA molecules with a stem-loop structure, since a portion of the single strand constitutes a strand complementary to another portion. Thus, molecules capable of forming an intramolecular RNA duplex structure are also included in the siRNAs of the present invention.

In a preferred embodiment of the present invention, the siRNAs of the present invention also include, for example, double-stranded RNAs with additions or deletions of one or a few RNAs in an siRNA which targets a specific DNA sequence (SEQ ID NOs: 67 to 70) shown herein and which can suppress the expression of versican, C4ST-1, C4ST-2, C4ST-3, or such via RNAi effect, as long as the double-stranded RNAs have the function of suppressing the expression of a gene encoding an above-described CSPG core protein, synthetase, desulfation enzyme-suppressing protein, or sulfotransferase.

The RNAs used in RNAi (siRNAs) do not need to be perfectly identical (homologous) to the genes encoding the above proteins or portions thereof; however, the RNAs are preferably perfectly identical (homologous).

Some details of the RNAi mechanism still remain unclear, but it is understood that an enzyme called “DICER” (a member of the RNase III nuclease family) is contacted with a double-stranded RNA and degrades it in to small fragments, called “small interfering RNAs” or “siRNAs”. The double-stranded RNAs of the present invention that have RNAi effect include such double-stranded RNAs prior to being degraded by DICER. Specifically, since even long RNAs that have no RNAi effect when intact can be degraded into siRNAs which have RNAi effect in cells, the length of the double-stranded RNAs of the present invention is not particularly limited.

For example, long double-stranded RNAs covering the full-length or near full-length mRNA of a gene encoding an above-described CSPG core protein, synthetase, desulfation enzyme-suppressing protein, or sulfotransferase can be pre-digested, for example, by DICER, and then the degradation products can be used as agents of the present invention. These degradation products are expected to contain double-stranded RNA (siRNA) molecules with RNAi effect. With this method, it is not necessary to specifically select the mRNA regions expected to have RNAi effect. In other words, it is not necessary to accurately determine regions with RNAi effect in the mRNAs of the genes described above.

The above-described “double-stranded RNAs capable of suppression via RNAi effect” can be suitably prepared by those skilled in the art based on nucleotide sequences of the above-described CSPG genes encoding core proteins, synthetases, desulfation enzyme-suppressing proteins, or sulfotransferases, which are targeted by the double-stranded RNAs. For example, the double-stranded RNAs of the present invention can be prepared based on the nucleotide sequence of SEQ ID NO: 67. In other words, it is within the range of ordinary trials for those skilled in the art to select an arbitrary consecutive RNA region in an mRNA that is a transcript of the nucleotide sequence of SEQ ID NO: 67, and prepare double-stranded RNA corresponding to the region. Those skilled in the art can also use known methods to properly select siRNA sequences with stronger RNAi effect from the mRNA sequence, which is the transcript of the nucleotide sequence of SEQ ID NO: 67. When one of the strands is already identified, those skilled in the art can readily determine the nucleotide sequence of the other strand (complementary strand). Those skilled in the art can appropriately prepare siRNAs using a commercially available nucleic acid synthesizer. Alternatively, general custom synthesis services may be used to synthesize desired RNAs.

The siRNAs of the present invention are not necessarily single pairs of double-stranded RNAs directed to target sequences, but may be mixtures of multiple double-stranded RNAs directed to regions that cover the target sequence. Herein, those skilled in the art can appropriately prepare the siRNAs as nucleic acid mixtures matched to a target sequence by using a commercially available nucleic acid synthesizer or DICER enzyme. Meanwhile, general custom synthesis services may be used to synthesize desired RNAs. The siRNAs of the present invention include so-called “siRNA cocktails”.

All nucleotides in the siRNAs of the present invention do not necessarily need to be ribonucleotides (RNAs). Specifically, one or more of the ribonucleotides constituting the siRNAs of the present invention may be replaced with corresponding deoxyribonucleotides. The term “corresponding” means that although the sugar moieties are structurally differently, the nucleotide residues (adenine, guanine, cytosine, or thymine (uracil)) are the same. For example, deoxyribonucleotides corresponding to ribonucleotides with adenine refer to deoxyribonucleotides with adenine. The term “or more” described above is not particularly limited, but preferably refers to a small number of about two to five ribonucleotides.

Furthermore, DNAs (vectors) capable of expressing the RNAs of the present invention are also included in the preferred embodiments of compounds capable of suppressing the expression of the genes encoding the above-described proteins of the present invention. The DNAs (vectors) capable of expressing the double-stranded RNAs of the present invention are, for example, DNAs structured such that a DNA encoding one strand of a double-stranded RNA and a DNA encoding the other strand of the double-stranded RNA are linked with promoters so that each DNA can be expressed. The above DNAs of the present invention can be appropriately prepared by those skilled in the art using standard genetic engineering techniques. More specifically, the expression vectors of the present invention can be prepared by adequately inserting DNAs encoding the RNAs of the present invention into various known expression vectors.

Furthermore, the expression-inhibiting substances of the present invention also include compounds that inhibit the expression of the above-described CSPG core proteins, synthetases, desulfation enzyme-suppressing proteins, or sulfotransferases by binding to an expression regulatory region of a gene encoding the above-described CSPG core proteins, synthetases, desulfation enzyme-suppressing proteins, or sulfotransferases (for example, a promoter region; specific examples include the nucleotide sequence of SEQ ID NO: 66, which is a promoter region of PG-Lb). Such compounds can be obtained, for example, using a fragment of a promoter DNA of the gene encoding an above-described CSPG core protein, synthetase, desulfation enzyme-suppressing protein, or sulfotransferase to perform screening methods using as an indicator the activity of binding to the DNA fragment. Those skilled in the art can appropriately determine whether compounds of interest inhibit the expression of the above-described genes encoding CSPG core proteins, synthetases, desulfation enzyme-suppressing proteins, or sulfotransferases by using known methods, for example, reporter assays and such.

Furthermore, DNAs (vectors) capable of expressing the above-described RNAs of the present invention are also included in preferred embodiments of the compounds capable of inhibiting the expression of a gene encoding an above-described CSPG core protein, synthetase, desulfation enzyme-suppressing protein, or sulfotransferase of the present invention. For example, DNAs (vectors) capable of expressing the above-described double-stranded RNAs of the present invention are structured such that a DNA encoding one strand of a double-stranded RNA and a DNA encoding the other strand of the double-stranded RNA are linked to promoters so that both can be expressed. Those skilled in the art can appropriately prepare the above-described DNAs of the present invention using standard genetic engineering techniques. More specifically, the expression vectors of the present invention can be prepared by appropriately inserting DNAs encoding the RNAs of the present invention into various known expression vectors.

Preferred embodiments of the above-described vector of the present invention include vectors expressing RNAs (siRNAs) that can suppress the expression of versican, C4ST-1, C4ST-2, C4ST-3, or the like by RNAi effect.

Antibodies that bind to the above-described CSPG core proteins, synthetases, desulfation enzyme-suppressing compounds, or sulfotransferases can be prepared by methods known to those skilled in the art. Polyclonal antibodies can be obtained, for example, by the following procedure: small animals such as rabbits are immunized with an above-described natural protein or a recombinant protein expressed in microorganisms as a fusion protein with GST, or a partial peptide thereof. Sera are obtained from these animals and purified by, for example, ammonium sulfate precipitation, Protein A or G column, DEAE ion exchange chromatography, affinity column coupled with the core protein, synthetase, desulfation enzyme-suppressing compound, or sulfotransferase for CSPGs described above, synthetic peptide, or such, to prepare antibodies. Monoclonal antibodies can be obtained by the following procedure: small animals such as mice are immunized with an above-described CSPG core protein, synthetase, desulfation enzyme-suppressing compound, or sulfotransferase, or a partial peptide thereof. Spleens are removed from the mice and crushed to isolate cells. The cells are fused with mouse myeloma cells using a reagent such as polyethylene glycol. Clones producing antibodies that bind to an above-described CSPG core protein, synthetase, desulfation enzyme-suppressing compound, or sulfotransferase are selected from among the resulting fused cells (hybridomas). The obtained hybridomas are then transplanted in the peritoneal cavities of mice, and ascites is collected from the mice. The obtained monoclonal antibodies can be purified by, for example, ammonium sulfate precipitation, Protein A or G columns, DEAE ion exchange chromatography, affinity columns coupled with an above-described CSPG core protein, synthetase, desulfation enzyme-suppressing compound, or sulfotransferase, synthetic peptides, or such.

The antibodies of the present invention are not particularly limited as long as they bind to an above-described core protein, synthetase, desulfation enzyme-suppressing compound, or sulfotransferase of the present invention. The antibodies of the present invention may be human antibodies, humanized antibodies created by gene recombination, fragments or modified products of such antibodies, in addition to the polyclonal and monoclonal antibodies described above.

The proteins of the present invention used as sensitizing antigens to prepare antibodies are not limited in terms of the animal species from which the proteins are derived. However, the proteins are preferably derived from mammals, for example, mice and humans. Human-derived proteins are particularly preferred. The human-derived proteins can be appropriately obtained by those skilled in the art using the gene or amino acid sequences disclosed herein.

In the present invention, the proteins to be used as sensitizing antigens may be whole proteins or partial peptides thereof. Such partial peptides of the proteins include, for example, amino-terminal (N) fragments and carboxyl-terminal (C) fragments of the proteins. Herein, “antibodies” refer to antibodies that react with a full-length protein or fragment thereof.

In addition to immunizing nonhuman animals with antigens to obtain the above hybridomas, human lymphocytes, for example, EB virus-infected human lymphocytes, can be sensitized in vitro with the proteins or with cells expressing the proteins, or with lysates thereof, and the sensitized lymphocytes can be fused with human-derived myeloma cells with the ability to divide permanently, for example, U266, to obtain hybridomas that produce desired human antibodies with binding activity to the proteins.

It is expected that antibodies against the above-described CSPG core proteins, synthetases, desulfation enzyme-suppressing compounds, or sulfotransferases of the present invention exhibit the effect of inhibiting protein expression or function by binding to the proteins. When using the prepared antibodies for human administration (antibody therapy), the antibodies are preferably human or humanized antibodies in order to reduce immunogenicity.

Furthermore, in the present invention, low-molecular-weight substances (low-molecular-weight compounds) that bind to the above-described CSPG core proteins, synthetases, desulfation enzyme-suppressing compounds, or sulfotransferases are also included in the substances capable of inhibiting the function of the above-described CSPG core proteins, synthetases, desulfation enzyme-suppressing compounds, or sulfotransferases. Such low-molecular-weight substances may be natural or artificial compounds. In general, the compounds can be produced or obtained by methods known to those skilled in the art. The compounds of the present invention can also be obtained by the screening methods described below.

In addition, the substances of the present invention capable of inhibiting the expression or function of the above-described CSPG core proteins, synthetases, desulfation enzyme-suppressing proteins, or sulfotransferases include dominant-negative mutants (dominant-negative proteins) for the above-described CSPG core proteins, synthetases, desulfation enzyme-suppressing proteins, or sulfotransferases. The “dominant-negative protein mutants for the above CSPG core proteins, synthetases, desulfation enzyme-suppressing proteins, or sulfotransferases” refer to proteins with the function of reducing or abolishing the activity of endogenous wild-type proteins by expressing the genes encoding the CSPG core proteins, synthetases, desulfation enzyme-suppressing proteins, or sulfotransferases. Such dominant-negative proteins include, for example, versican core protein mutants that competitively inhibit the linking of the wild-type versican core protein with chondroitin sulfate.

Furthermore, in the present invention, the organ where the production or accumulation of chondroitin sulfate proteoglycans is inhibited is preferably the intestine, and is more preferably the large intestine or the small intestine.

Compounds that inhibit the production or accumulation of chondroitin sulfate proteoglycans are expected to serve as therapeutic or preventive agents for intestinal inflammation. Herein, “therapeutic or preventive” does not necessarily refer to a perfect therapeutic or preventive effect on intestinal inflammation, and may refer to a partial effect.

In the present invention, the intestinal inflammation is not specifically limited; however, the intestinal inflammation is preferably inflammatory bowel disease, and more preferably ulcerative colitis or Crohn's disease.

The intestinal inflammation-suppressing agents of the present invention have the activity of suppressing intestinal inflammation through inhibiting the production or accumulation of chondroitin sulfate proteoglycans, which is a cause of intestinal inflammation. Thus, the present invention provides therapeutic agents for ulcerative bowel disease and Crohn's disease, which comprise as an active ingredient an intestinal inflammation-suppressing agent of the present invention.

The “intestinal inflammation-suppressing agents” of the present invention can also be referred to as “therapeutic agents for intestinal inflammation”, “intestinal inflammation-improving agents”, “anti-intestinal inflammation agents”, or the like. Meanwhile, the “suppressing agents” of the present invention can also be referred to as “pharmaceutical agents”, “pharmaceutical compositions”, “therapeutic medicines”, or the like.

The “treatments” of the present invention also comprise improving effects, preventive effects, and the like, that can suppress the onset of intestinal inflammation in advance. The treatments are not limited to those producing a perfect therapeutic effect on cells (tissues) developing intestinal inflammation, and the effects may be partial.

The agents of the present invention can be combined with physiologically acceptable carriers, excipients, diluents and such, and orally or parenterally administered as pharmaceutical compositions. Oral agents may be in the form of granules, powders, tablets, capsules, solutions, emulsions, suspensions, or the like. The dosage forms of parenteral agents can be selected from injections, infusions, external preparations, suppositories, and the like. Injections include preparations for subcutaneous, intramuscular, and intraperitoneal injections, and the like. The external preparations include nasal preparations, ointments, and such. Techniques for formulating the above-described dosage forms that contain the agents of the present invention as primary ingredients are known.

For example, tablets for oral administration can be produced by compressing and shaping the agents of the present invention in combination with excipients, disintegrants, binders, lubricants, and the like. Excipients commonly used include lactose, starch, mannitol, and the like. Commonly used disintegrants include calcium carbonate, carboxymethylcellulose calcium, and the like. Binders include gum arabic, carboxymethylcellulose, and polyvinylpyrrolidone. Known lubricants include talc, magnesium stearate, and such.

Known coatings can be applied to tablets comprising the agents of the present invention to prepare enteric coated formulations or for masking. Ethylcellulose, polyoxyethylene glycol, or such can be used as a coating agent.

Meanwhile, injections can be prepared by dissolving the agents of the present invention, which are chief ingredients, together with an appropriate dispersing agent, or dissolving or dispersing the agents in a dispersion medium. Both water-based and oil-based injections can be prepared, depending on the selection of dispersion medium. When preparing water-based injections, the dispersing agent is distilled water, physiological saline, Ringer's solution or such. For oil-based injections, any of the various vegetable oils, propylene glycols, or such is used as a dispersing agent. If required, a preservative such as paraben may be added at this time. Known isotonizing agents such as sodium chloride and glucose can also be added to the injections. In addition, soothing agents such as benzalkonium chloride and procaine hydrochloride can be added.

Alternatively, the agents of the present invention can be formed into solid, liquid, or semi-solid compositions to prepare external preparations. Such solid or liquid compositions can be prepared as the same compositions as described above and then used as external preparations. The semi-solid compositions can be prepared using an appropriate solvent, to which a thickener is added if required. Water, ethyl alcohol, polyethylene glycol, and the like can be used as the solvent. Commonly used thickeners are bentonite, polyvinyl alcohol, acrylic acid, methacrylic acid, polyvinylpyrrolidone, and the like. Preservatives such as benzalkonium chloride can be added to these compositions. Alternatively, suppositories can be prepared by combining the compositions with carriers, like oil bases such as cacao butter, or aqueous gel bases such as cellulose derivatives.

When the agents of the present invention are used as gene therapy agents, the agents may be directly administered by injection, or vectors carrying the nucleic acid may be administered. Such vectors include adenovirus vectors, adeno-associated virus vectors, herpes virus vectors, vaccinia virus vectors, retroviral vectors, and lentivirus vectors. These vectors allow efficient administration.

Alternatively, the agents of the present invention can be encapsulated into phospholipid vesicles such as liposomes, and then the vesicles can be administered. Vesicles carrying siRNAs or shRNAs are introduced into given cells by lipofection. The resulting cells are then systemically administered, for example, intravenously or intra-arterially. The cells can also be locally administered into tissues or such with intestinal inflammation. siRNAs exhibit a quite superior and specific post-transcriptional suppression effect in vitro; however, in vivo they are rapidly degraded due to serum nuclease activity, and thus, their was limited. There is therefore demand for the development of optimized and effective delivery systems. Atelocollagen, which is a representative example of an siRNA carrier, is a non-antigenic molecule obtained by digesting collagen molecule with pepsin. Atelocollagen has high bioaffinity, strongly interacts with nucleic acids, has no risk of inducing intestinal inflammation when administered into the body, and is biodegradable in vivo. Atelocollagen therefore has drawn attention as a useful carrier for in vivo gene vectors (Ochiya, T., Nature Med. 1999, 5(6), 707-10). However, the present invention's method for introducing pharmaceutical agents is not limited to the method using atelocollagen.

The agents of the present invention are administered to mammals including humans at required (effective) doses, within a dose range considered to be safe. Ultimately, the doses of the agents of the present invention can be appropriately determined by medical practitioners or veterinarians after considering the dosage form and administration method, and the patient's age and weight, symptoms, and the like. For example, adenoviruses are administered once a day at a dose of about 106 to 1013 viruses every one to eight weeks, although the doses vary depending on the age, sex, symptoms, administration route, administration frequency, and dosage form.

Commercially available gene transfer kits (for example: AdenoExpress™, Clontech) may be used to introduce siRNAs or shRNAs into target tissues or organs.

When the agents of the present invention are used, the type of disease and site to which the agents are applied are not particularly limited, as long as the disease develops intestinal inflammation; for example, the agents are applied to ulcerative colitis, Crohn's disease and such. The above diseases may occur in combination with other diseases.

The present invention also provides methods of screening for intestinal inflammation-suppressing agents, wherein the methods comprise selecting, from test samples, substances with the activity of inhibiting the production or accumulation of chondroitin sulfate proteoglycans. Intestinal inflammation-suppressing agents or candidate compounds for intestinal inflammation-suppressing agents can be efficiently obtained using the screening methods of the present invention.

Preferred embodiments of the screening methods of the present invention are methods of screening for intestinal inflammation-suppressing agents that comprise the step of selecting substances with any of the activities (a) to (d):

(a) promoting the degradation of chondroitin sulfate proteoglycans;
(b) inhibiting the synthesis of chondroitin sulfate proteoglycans;
(c) desulfating chondroitin sulfate proteoglycans; and
(d) inhibiting the sulfation of chondroitin sulfate proteoglycans.

Representative examples based on fundamental principles common in screening for these substances include methods comprising the following procedure: A preferred procedure uses tools (1) to (3) below, and is as follows: (1) and (2) are mixed in a test tube or culture dish, and the resulting effect is simply detected using (3).

(1) chondroitin sulfate proteoglycans (CSPGs) themselves, or glycosaminoglycans (GAG) chains, or cells synthesizing (producing) CSPGs or GAG chains
(2) test compounds (for example, enormous compound libraries owned by pharmaceutical companies)
(3) methods for detecting CSPG cleavage sites, the amount of CSPGs, or the amount of free glycosaminoglycans (GAGs)

Embodiments of the screening methods of the present invention are exemplified below. In the embodiments described below, the chondroitin sulfate proteoglycans, synthetases, compounds suppressing desulfation enzymes, sulfotransferases, degradation-promoting enzymes, and desulfation enzymes to be used include those derived from humans, mice, rats, and others, but are not limited thereto. Chondroitin sulfate proteoglycan portions are components such as glycosaminoglycan chains or core proteins, or portions thereof. The chondroitin sulfate proteoglycan portions are not particularly limited.

The test compounds to be used in the embodiments described below are not particularly limited, but include, for example, single compounds, such as natural compounds, organic compounds, inorganic compounds, proteins, and peptides, as well as compound libraries, expression products of gene libraries, cell extracts, cell culture supernatants, products of fermenting microorganisms, extracts of marine organisms, and plant extracts.

In the embodiments described below, the “contact” with test compounds is typically achieved by mixing the test compounds with chondroitin sulfate proteoglycans, portions thereof, synthetases, compounds suppressing desulfation enzymes, sulfotransferases, degradation-promoting enzymes, or desulfation enzymes, but the “contact” is not limited to this methods. For example, the “contact” can also be achieved by contacting test compounds with cells expressing these proteins or portions thereof.

In the embodiments described below, the “cells” include those derived from humans, mice, rats, and such, but are not limited thereto. Cells of microorganisms, such as Escherichia coli and yeasts, which are transformed to express the proteins used in each embodiment, can also be used. For example, the “cells that express chondroitin sulfate proteoglycans” include cells that express endogenous genes for chondroitin sulfate proteoglycans, and cells that express introduced foreign genes for chondroitin sulfate proteoglycans. Such cells that express foreign genes for chondroitin sulfate proteoglycans can typically be prepared by introducing host cells with expression vectors carrying a chondroitin sulfate proteoglycan gene as an insert. The expression vectors can be prepared using standard genetic engineering techniques.

The “chondroitin sulfate proteoglycan core proteins” described below include, for example, core proteins of matrix-type chondroitin sulfate proteoglycans, such as aggrecan, versican, neurocan, and brevican, and core proteins of membrane chondroitin sulfate proteoglycans, such as decorin, biglycan, fibromodulin, and PG-Lb. The “synthetases” include, for example, GalNAc4ST-1, GalNAc4ST-2, GALNAC4S-6ST, UA20ST, GalT-I, GalT-II, GlcAT-1, and XylosylT. The “sulfotransferases” include, for example, chondroitin D-N-acetylgalactosamine-4-O-sulfotransferase 1 (C4ST-1), chondroitin D-N-acetylgalactosamine-4-O-sulfotransferase 2 (C4ST-2), chondroitin D-N-acetylgalactosamine-4-O-sulfotransferase 3 (C4ST-3), D4ST, C6ST-1, and C6ST-2. The “degradation-promoting enzymes” include, for example, ADAMTS-1, ADAMTS-4, ADAMTS-5, chondroitinase ABC (ChABC), chondroitinase AC, chondroitinase B, and calpain I. The “desulfation enzymes” include, for example, chondroitin-4-sulfatase and chondroitin-6-sulfatase.

Embodiments of the screening methods of the present invention include methods comprising the step of selecting compounds that have the activity of promoting the degradation of chondroitin sulfate proteoglycans. An example of the above-mentioned methods of the present invention comprises the steps of:

(a) contacting test compounds with chondroitin sulfate proteoglycans or portions thereof;
(b) measuring the abundance of chondroitin sulfate proteoglycans or portions thereof; and
(c) selecting substances that reduce the abundances as compared with those determined in the absence of the test compounds.

In the above methods, first, test compounds are contacted with chondroitin sulfate proteoglycans or portions thereof.

In these methods, the amount of the chondroitin sulfate proteoglycans or portions thereof is then measured. The measurement can be conducted by methods known to those skilled in the art. For example, the amounts can be detected using labeled compounds or antibodies that bind to the chondroitin sulfate proteoglycans or portions thereof, and then measuring the amount of the label. Alternatively, the detection can be achieved by chromatography or mass spectrometry.

In these methods, compounds that reduce the abundance of the chondroitin sulfate proteoglycans or portions thereof as compared with in the absence of a test compound (the control) are then selected. Compounds resulting in a reduction can be used as therapeutic agents for intestinal inflammation.

Below is a brief illustrative example of the methods able to assess (measure) whether a test compound has the above activity (a): the activity of promoting the degradation of chondroitin sulfate proteoglycans.

Embodiment of the methods for screening for the above activity (a) of promoting the degradation of chondroitin sulfate proteoglycans:

A CS-GAG, such as chondroitin sulfate A (CS-A), CS-B, CS-C (Seikagaku Co., ICN, Sigma, and others), or human-derived proteoglycan (BGN, ISL, and others), is prepared, and 96-well plates are coated with it at a concentration of 10 μg/ml (using known methods, such as in Kawashima, H. et al., J. Biol. Chem. 2002, 277, 12921-12930). Various test compounds are added to each well of the plates. After two hours of reaction at 37° C., changes in CS-GAG are detected.

Detection methods include, for example, the simple WFA lectin (Wisteria floribunda lectin)-binding method. Since WFA lectin binds to the GalNAc residues of CS-GAG chains, it can easily detect CS-GAGs. Chondroitinase ABC is used as a positive control for test compounds. The principle behind this use of chondroitinase ABC is that its addition degrades CS-GAG chains, making it impossible for WFA lectin to bind them. More specifically, FITC-labeled WFA lectin (EY Co.) is added to the CS-coated wells before and after mixing the test compounds, and changes in the intensity of FITC fluorescence in the wells due to the CS-GAG degradation can be quantified and digitized very simply by using detection devices, such as fluorescence plate readers or fluorescence microscopes. Compounds whose addition most reduces fluorescent values may be determined to be novel therapeutic candidate compounds that fulfill the concept of the present invention.

In an alternative detection method, anti-CS antibody (clone CS56, Seikagaku Co.) can be used to directly label CS-GAGs. As with WFA lectin, large-scale screening can be carried out simply and in very short time by adding FITC-labeled anti-CS antibody to CS-coated wells and examining changes in fluorescence value.

In more specific detection methods, GAG content is accurately quantified and digitized by simply using the plates before and after mixing of test compounds in an sGAG Assay Kit (Wieslab Co.), an ELISA Kit for Sulphanated Glycosaminoglycans (Funakoshi Co.), or such.

More specifically, the reducing ends of free GAG chains can easily be fluorescently labeled by adding 2-aminobenzamide, 2-aminopyridine (2-AB and 2-AP, respectively; LUD Co. and others), or the like to the plates before and after mixing of test compounds, which enables more specific analysis using HPLC, MALDI-MS, LC-MS, or such to determine the types of sugar chains and even the content of each type of chain. These methods, which examine the properties of candidate compounds in detail, take screening to the next level.

Other embodiments of the screening methods of the present invention include methods comprising the step of selecting substances with the activity of inhibiting the synthesis of chondroitin sulfate proteoglycans. These methods of the present invention comprise, for example, the steps of:

(a) contacting test compounds with cells expressing chondroitin sulfate proteoglycans or portions thereof, extracts of these cells, or groups of substances including those enzymes and substrates constituting the process of chondroitin sulfate proteoglycan synthesis;
(b) measuring the amount of synthesized chondroitin sulfate proteoglycans or intermediates thereof in the above-mentioned cells, cell extracts, or group of substances; and
(c) selecting compounds that reduce the amount as compared to in the absence of the test compounds.

In the above methods, test compounds are first contacted with cells expressing chondroitin sulfate proteoglycans or portions thereof, extract of these cells, or groups of substances including those enzymes and substrates that constitute the process of chondroitin sulfate proteoglycan synthesis.

Next, the amount of synthesized chondroitin sulfate proteoglycans or intermediates thereof is measured. The measurement can be performed by those skilled in the art using known methods; for example, methods using labeled antibodies, mass spectrometry, and chromatography can be used.

Further, compounds that reduce (suppress) the synthesized amount as compared with in the absence of the test compounds (the control) are selected. Compounds resulting in a reduction (suppression) can be used as therapeutic agents for intestinal inflammation.

Below is a brief illustrative example of the methods able to assess (measure) whether a test compound has the above activity (b): the activity of inhibiting the synthesis of chondroitin sulfate proteoglycans.

Embodiment of the methods for screening for the above activity (b) of inhibiting the synthesis of chondroitin sulfate proteoglycans:

Cells and cell lines synthesizing chondroitin sulfate are known to researchers in the art. In human, for example, chondroitin sulfate is produced after 16 hours of cell culture by standard methods for culturing mononuclear cells isolated from peripheral blood collected from healthy subjects (Ulhlin-Hansen, L. et al., Blood 1993, 82, 2880; etc.). Alternatively, for more convenience, there are many examples of known cell; for example, the fibroblast cell line NIH3T3 (Phillip, H. A. et al., J. Biol. Chem. 2004, 279, 48640; etc.); the renal tubule-derived cancer cell line ACHN (Kawashima, H. et al., J. Biol. Chem. 2002, 277, 12921), the renal distal tubule-derived cell line MDCK (Borges, F. T. et al., Kidney Int. 2005, 68, 1630; etc.), and the vascular endothelial cell line HUVEC (Schick, B. P. et al., Blood 2001, 97, 449; etc.). Various test compounds are added during the process of culturing such cell lines for set periods, and changes in the amount of CS-GAG before and after culture can be easily evaluated by the above-described method of (a). Compounds that suppress the increase in the amount of CS-GAG after culture (which thus reflects the amount of synthesized CS-GAG) can be easily determined to be candidate therapeutic compounds that fulfill the concept of the present invention.

As a further option, cell lines constitutively expressing the genes for CS-GAG synthetases such as GalNAc4ST-1 and XylosylT can be prepared by introducing the genes into CHO cells, L cells, or such by well-known methods. The use of such cell lines that constitutively synthesize CS-GAG allows more clear determination of candidates for therapeutic compounds.

In another embodiment, the screening methods of the present invention include methods comprising the step of selecting substances with the activity of desulfating chondroitin sulfate proteoglycans. The above methods of the present invention comprise, for example, the steps of:

(a) contacting test compounds with chondroitin sulfate proteoglycans or portions thereof;
(b) measuring the amount of sulfation in the chondroitin sulfate proteoglycans or portions thereof; and
(c) selecting substances that reduce the amount of sulfation as compared with in the absence of the test compounds.

In the above methods, test compounds are first contacted with chondroitin sulfate proteoglycans or portions thereof.

Next, the amount of sulfation in the chondroitin sulfate proteoglycans or portions thereof is measured. The measurement can be conducted using methods known to those skilled in the art. For example, the amount of sulfation can be determined by using labeled compounds or antibodies that bind to the desulfated structures remaining in the chondroitin sulfate proteoglycans or portions thereof, and measuring the amount of the label. Alternatively, the measurement can be achieved by chromatography or mass spectrometry or such.

Then, in the present methods compounds that reduce the abundances of the chondroitin sulfate proteoglycans or portions thereof as compared with in the absence of the test compounds (the control) are selected. Compounds resulting in a reduction can be used as therapeutic agents for intestinal inflammation.

Below is a brief illustrative example of the methods able to assess (measure) whether a test compound has the above activity (c): the activity of desulfating chondroitin sulfate proteoglycans.

Embodiment of the methods for screening for the above activity (c) of desulfating chondroitin sulfate proteoglycans:

By using essentially the same method as described above in (a), human-derived proteoglycans (BGN Co., ISL Co., etc.) or such are prepared and coated on to 6-well plates at a concentration of 10 μg/ml (by known methods, such as those described in Kawashima, H. et al., J. Biol. Chem. 2002, 277, 12921-12930). Various test compounds are added to each well of the plates, and alterations in CS-GAG are detected after two hours of reaction at 37° C.

In this detection method, desulfated moieties can be easily detected using the reaction of either anti-proteoglycan Δdi4S antibody (clone: 2-B-6, which recognizes sulfated moieties at position 4) or anti-proteoglycan Δdi6S antibody (clone: 3-B-3, which recognizes sulfated moieties at position 6) (both from Seikagaku Co.) with the disaccharide structure of the desulfated fragments that remain in the core protein of proteoglycans after desulfation. Thus, FITC-labeled 2-B-6 or 3-B-3 antibody is reacted in such plates before and after mixed culture, and changes in the fluorescence value can be simply detected. Compounds whose addition increases the fluorescence intensity can be determined to be substances that promote desulfation, and are easily identified as novel candidate therapeutic compounds that fulfill the concept of the present invention.

Another embodiment of the screening methods of present invention includes methods comprising the step of selecting substances with the activity of inhibiting the sulfation of chondroitin sulfate proteoglycans. The above methods of the present invention comprise, for example, the steps of:

(a) contacting test compounds with cells expressing chondroitin sulfate proteoglycans or portions thereof, extracts of these cells, or groups of substances including those enzymes and substrates constituting the process of sulfation of chondroitin sulfate proteoglycans;
(b) measuring the activity of sulfation of chondroitin sulfate proteoglycans in the above-mentioned cells, cell extracts, or groups of substances; and
(c) selecting compounds that reduce the activity as compared with in the absence of the test compounds.

In the above methods, test compounds are first contacted with chondroitin sulfate proteoglycans or portions thereof.

Next, the amount of sulfation in the chondroitin sulfate proteoglycans or portions thereof is measured. The measurement can be conducted using methods known to those skilled in the art. For example, the amount of sulfation can be determined by using labeled compounds or antibodies that bind to the sulfated structures of the chondroitin sulfate proteoglycans or portions thereof, and measuring the amount of the label. Alternatively, the measurement can be achieved by chromatography or mass spectrometry and such.

Then, compounds that reduce the abundance of the chondroitin sulfate proteoglycans or portions thereof as compared with in the absence of the test compounds (the control) are selected. Compounds resulting in a reduction can be used as therapeutic agents for intestinal inflammation.

Below is a brief illustrative example of the methods able to assess (measure) whether a test compound has the above activity (d): the activity of inhibiting the sulfation of chondroitin sulfate proteoglycans.

Embodiment of the methods for screening for the above activity (d) of inhibiting the sulfation of chondroitin sulfate proteoglycans:

The cells and cell lines that promote the sulfation of chondroitin sulfates are the same as described above in (c). Various test compounds are mixed during a set period of culture of such cell lines, and the degree of sulfation before and after the culture can be easily determined by, for example, using an antibody that recognizes sulfation at position 4 (clone: LY111) or an antibody that recognizes sulfation at position 6 (clone: MC21C) (both from Seikagaku Co.). Fluorescence values may be compared between before and after the culture by using fluorescently labeled antibodies. Alternatively, the same detection method as described above in (c) can be conducted using 2-B-6 or 3-B-3 antibodies before and after culture. Compounds that suppress an increase in the sulfation after cell culture (an increase in the fluorescence value for LY111 or MC21C), or compounds that promote the progression of desulfation after cell culture (an increase in the fluorescence value for 2-B-6 or 3-B-3) can be easily determined to be candidate therapeutic compounds that fulfill the concept of the present invention.

As a further option, cell lines that constitutively express sulfotransferase genes such as C4ST-1 and C6ST-1 can be prepared by introducing the genes into CHO cells, L cells, or such by well-known methods. The use of such cell lines that constitutively add sulfate groups allows more clear determination of candidates for therapeutic compounds.

Other preferred embodiments of the present invention are methods of screening for intestinal inflammation-suppressing agents in which compounds that reduce the expression level of a gene encoding a CSPG core protein, synthetase, desulfation enzyme-suppressing protein, or sulfotransferase of the present invention, or compounds that increase the expression level of a gene for an enzyme that desulfates CSPGs or promotes the degradation of CSPGs, are selected; wherein the method comprises the steps of:

(a) contacting test compounds with cells expressing a gene encoding a CSPG core protein, synthetase, desulfation enzyme-suppressing protein, sulfotransferase, degradation-promoting enzyme, or desulfating enzyme;
(b) determining the expression level of the gene in the cells;
(c) comparing the expression level with that in the absence of the test compounds (the control); and
(d) selecting compounds that reduce the expression level of the gene of the CSPG core protein, synthetase, desulfation enzyme-suppressing protein, or sulfotransferase as compared with the control, or compounds that increase the expression level of the gene of the CSPG desulfating enzyme or the CSPG degradation-promoting enzyme as compared with the control.

In the above methods, test compounds are first contacted with cells expressing a gene encoding a CSPG core protein, synthetase, desulfating enzyme-suppressing protein, sulfotransferase, degradation-promoting enzyme, or desulfating enzyme.

Next, the expression level of the gene encoding the core protein, synthetase, desulfating enzyme-suppressing protein, sulfotransferase, degradation-promoting enzyme, or desulfating enzyme is measured. Herein, “expression of the gene” includes both transcription and translation. Gene expression level can be measured by methods known to those skilled in the art.

For example, mRNAs are extracted from cells expressing any one of the above-described proteins by conventional methods, and these mRNAs can be used as templates in Northern hybridization, RT-PCR, DNA arrays, or such to measure the transcription level of the gene. Alternatively, protein fractions are collected from cells expressing a gene encoding any of the above-described proteins, and expression of the protein can be detected by electrophoresis such as SDS-PAGE to measure the level of gene translation. Alternatively, the level of gene translation can be measured by detecting the expression of any of the above-described proteins by Western blotting using an antibody against the proteins. Such antibodies for use in detecting the proteins are not particularly limited, as long as they are detectable. For example, both monoclonal and polyclonal antibodies can be used.

Next, the expression level is compared with that in the absence of the test compounds (the control).

Then, when the gene encodes a CSPG core protein, synthetase, desulfating enzyme-suppressing protein, or sulfotransferase, compounds that reduce (suppress) the expression level of the gene as compared with a control are selected. The compounds resulting in a reduction (suppression) can be agents for suppressing intestinal inflammation or candidate compounds for treating intestinal inflammation.

Alternatively, when the gene encodes a CSPG desulfating enzyme or an enzyme promoting CSPG degradation, compounds that increase (enhance) the expression level of the gene as compared with a control are selected. Compounds resulting in an increase (enhancement) can be agents for suppressing intestinal inflammation or candidate compounds for treating intestinal inflammation.

An embodiment of the screening methods of the present invention is a method in which compounds that reduce the expression level of a gene encoding a CSPG core protein, synthetase, desulfation enzyme-suppressing protein, or sulfotransferase of the present invention, or compounds that increase the expression level of a gene for a CSPG degradation-promoting enzyme or a CSPG desulfating enzyme, can be selected using the expression of a reporter gene as an indicator. The above methods of the present invention comprise, for example, the steps of:

(a) contacting test compounds with cells or cell extracts containing a DNA structured such that a reporter gene is operably linked to a transcriptional regulatory region of a gene encoding a CSPG core protein, synthetase, desulfating enzyme-suppressing protein, sulfotransferase, degradation-promoting enzyme, or desulfating enzyme;
(b) measuring the expression level of the reporter gene;
(c) comparing the level with the that in the absence of the test compounds (the control); and
(d) selecting compounds that reduce the expression level of the reporter gene as compared with the control when the reporter gene is operably linked with a transcriptional regulatory region of the gene encoding a CSPG core protein, synthetase, desulfating enzyme-suppressing protein, or sulfotransferase; or selecting compounds that increase the expression level of the reporter gene as compared with the control when the reporter gene is operably linked with a transcriptional regulatory region of a gene encoding a CSPG degradation-promoting enzyme or a CSPG desulfating enzyme.

In the above methods, test compounds are first contacted with cells or cell extracts containing DNAs structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme.

Herein, “operably linked” means that a reporter gene is linked with a transcriptional regulatory region of a gene encoding a CSPG core protein, synthetase, desulfating enzyme-suppressing protein, sulfotransferase, degradation-promoting enzyme, or desulfating enzyme, such that expression of the reporter gene is induced upon binding of transcriptional factors to the transcriptional regulatory region. Therefore, the meaning of “operably linked” also includes cases where a reporter gene is linked with a different gene and produces a fusion protein with a different gene product, as long as expression of the fusion protein is induced upon the binding of transcriptional factors to the transcriptional regulatory region of the gene encoding the CSPG core protein, synthetase, desulfating enzyme-suppressing protein, sulfotransferase, degradation-promoting enzyme, or desulfating enzyme. Those skilled in the art can obtain the transcriptional regulatory regions of genes encoding CSPG core proteins, synthetases, desulfating enzyme-suppressing proteins, sulfotransferases, degradation-promoting enzymes, or desulfating enzymes that are present in the genome, based on the cDNA nucleotide sequences of the genes encoding the CSPG core proteins, synthetases, desulfating enzyme-suppressing proteins, sulfotransferases, degradation-promoting enzymes, or desulfating enzymes.

The reporter genes for use in these methods are not particularly limited, as long as their expression is detectable. The reporter genes include, for example, the CAT gene, the lacZ gene, the luciferase gene, and the GFP gene. The “cells containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme” include, for example, cells introduced with vectors carrying such structures as inserts. Such vectors can be prepared by methods well known to those skilled in the art. The vectors can be introduced into cells by standard methods, for example, calcium phosphate precipitation, electroporation, lipofection, and microinjection. The “cells containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme” include cells in which the structure has been integrated into the chromosomes. A DNA structure can be integrated into chromosomes by methods generally used by those skilled in the art, for example, gene transfer methods using homologous recombination.

The “cell extracts containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme” include, for example, mixtures of cell extracts included in commercially available in vitro transcription-translation kits and DNAs structured such that a reporter gene is operably linked with the transcriptional regulatory region of the gene encoding a CSPG core protein, synthetase, desulfating enzyme-suppressing protein, sulfotransferase, degradation-promoting enzyme, or desulfating enzyme.

“Contact” can be achieved by adding test compounds to a culture medium of “cells containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme”, or by adding test compounds to the above-described commercially available cell extracts containing the DNAs. When the test compound is a protein, contact may also be achieved, for example, by introducing a DNA vector expressing the protein into the cells.

In the above methods, the expression level of the reporter gene is then measured. The expression level of the reporter gene can be measured by methods known to those skilled in the art, depending on the type of the reporter gene. When the reporter gene is the CAT gene, its expression can be determined, for example, by detecting the acetylation of chloramphenicol by the gene product. When the reporter gene is the lacZ gene, its expression level can be determined by detecting the color development of chromogenic compounds due to the catalytic action of the gene expression product. Alternatively, when the reporter gene is the luciferase gene, its expression level can be determined by detecting the fluorescence of fluorogenic compounds due to the catalytic action of the gene expression product. Furthermore, when the reporter gene is the GFP gene, its expression level can be determined by detecting the fluorescence of the GFP protein.

In the above methods, the expression level of the reporter gene is then compared with that in the absence of the test compounds (the control).

In the present methods, compounds that reduce (suppress) the expression level of a reporter gene as compared with a control are then selected, where the reporter gene is operably linked with a gene encoding a CSPG core protein, synthetase, desulfating enzyme-suppressing protein, or sulfotransferase. Compounds resulting in a reduction (suppression) can be agents for suppressing intestinal inflammation or candidate compounds for treating intestinal inflammation.

Alternatively, when the reporter gene is operably linked with a gene encoding a CSPG degradation-promoting enzyme or CSPG desulfating enzyme, compounds that increase (enhance) the reporter gene expression level as compared with a control are selected. Compounds resulting in an increase (enhancement) can be agents for suppressing intestinal inflammation or candidate compounds for treating intestinal inflammation.

The intestinal inflammation-suppressing agents that are found by the screening methods of the present invention are preferably therapeutic or preventive agents for inflammatory bowel diseases.

The present invention also provides kits comprising various agents, reagents, and the like, which are used to conduct the screening methods of the present invention.

The kits of the present invention can be prepared, for example, by selecting adequate reagents from the above-described various reagents, depending on the screening method to be conducted. The kits of the present invention may contain, for example, the chondroitin sulfate proteoglycans of the present invention. The kits of the present invention may further contain various reagents, vessels, and the like to be used in the methods of the present invention. The kits may appropriately contain, for example, anti-chondroitin sulfate proteoglycan antibodies, probes, various reaction reagents, cells, culture media, control samples, buffers, and instruction manuals containing a description of how to use the kits.

Preferred embodiments of the present invention are the methods of screening for intestinal inflammation-suppressing agents, comprising the step of detecting whether the production or accumulation of chondroitin sulfate proteoglycans is inhibited. Thus, the kits for screening for the intestinal inflammation-suppressing agents of the present invention may contain, for example, oligonucleotides such as probes for the genes encoding CSPG core proteins, and primers to amplify certain regions of these genes; and antibodies recognizing CSPGs (anti-chondroitin sulfate proteoglycan antibodies), which can be used to detect chondroitin sulfate proteoglycans.

The above-described oligonucleotides specifically hybridize to, for example, DNAs of the genes encoding the versican core protein of the present invention. Herein, “specifically hybridize to” means that the oligonucleotides do not significantly cross-hybridize to DNAs encoding other proteins under standard hybridization conditions, and preferably under stringent hybridization conditions (for example, the conditions described in Sambrook, J. et al. “Molecular Cloning” 2nd Ed., Cold Spring Harbour Laboratory Press, New York, USA, 1989). The oligonucleotides are not necessarily perfectly complementary to the nucleotide sequences of the versican core protein genes of the present invention, as long as they allow specific hybridization.

The hybridization conditions in the present invention include, for example, conditions such as “2×SSC, 0.1% SDS, and 50° C.”, “2×SSC, 0.1% SDS, and 42° C.”, and “1×SSC, 0.1% SDS, and 37° C.”, and more stringent conditions such as “2×SSC, 0.1% SDS, and 65° C.”. “0.5×SSC, 0.1% SDS, and 42° C.”, and “0.2×SSC, 0.1% SDS, and 65° C.”. More specifically, for methods using Rapid-hyb buffer (Amersham Life Science), prehybridization is carried out at 68° C. for 30 minutes or more; then a probe is added, and after one hour or more of hybrid formation at 68° C., washing is carried out three times with 2×SSC/0.1% SDS at room temperature for 20 minutes, then three times with 1×SSC/0.1% SDS at 37° C. for 20 minutes, and finally twice with 1×SSC/0.1% SDS at 50° C. for 20 minutes. Alternatively, for example, prehybridization is carried out in ExpressHyb Hybridization Solution (CLONTECH) at 55° C. for 30 minutes, and then a labeled probe is added thereto, and after one hour or more of incubation at 37-55° C., washing is carried out three times in 2×SSC/0.1% SDS at room temperature for 20 minutes and then once in 1×SSC/0.1% SDS at 37° C. for 20 minutes. More stringent conditions can be achieved, for example, by increasing the temperature for prehybridization, hybridization, or second washing. For example, temperatures for prehybridization and hybridization can be 60° C., and more stringent conditions can be achieved by increasing the temperature to 68° C. In addition to conditions such as the salt concentration of the buffers and the temperature, those skilled in the art can also determine conditions using other conditions including the nucleotide sequence composition of the probe, the probe length and concentration, and reaction time.

The oligonucleotides can be used as probes and primers in the above-described screening kits of the present invention. When the oligonucleotides are used as primers, they are typically 15 to 100 bp long, and preferably 17 to 30 bp long. Such primers are, for example, those of SEQ ID NO: 71 or 72, but they are not particularly limited as long as they can amplify at least a portion of a DNA of the above-described genes of the present invention.

The present invention also provides therapeutic or preventive methods for diseases accompanying intestinal inflammation, which comprise the step of administering the agents of the present invention to individuals (for example, to patients and such).

The individuals subjected to the therapeutic or preventive methods of the present invention are not particularly limited, as long as they are organisms that can develop a disease accompanying intestinal inflammation; however, humans are preferred.

In general, administration to individuals can be achieved, for example, by methods known to those skilled in the art, such as intraarterial injections, intravenous injections, and subcutaneous injections. The administered dose varies depending on the patient's weight and age, and the administration method or such; however, those skilled in the art (medical practitioners, veterinarians, pharmacists, and the like) can appropriately select a suitable dose.

The present invention also relates to the uses of agents of the present invention in producing intestinal inflammation-suppressing agents.

All prior-art documents cited herein are incorporated by reference herein.

### EXAMPLES

Herein below, the present invention will be specifically described with reference to Examples, but the technical scope of the present invention is not to be construed as being limited thereto.

### Example 1

### Therapeutic Effects of Versican siRNA in Ulcerative Colitis Model Mice: Clinical Features

An ulcerative colitis model was prepared by allowing C57BL/6JcL mice (female, 6 weeks old; CLEA Japan Inc.) to freely drink high-concentration chlorine water containing 3% dextran sulfate sodium (DSS; Wako Pure Chemical Industries Ltd.) for eight days.

This DSS-induced ulcerative colitis model has excellent reproducibility, and is thus used widely in experimental systems for mouse ulcerative colitis (Sasaki, N., J Inflamm. 2005, 2, 13). At the same time when the mice were fed with 3% DSS water, 200 μl of a versican siRNA cocktail (5′-ATGAAAGGCATCTTATGGATGTGCTCA-3′ (SEQ ID NO: 67), 5′-ATTACTAACCCATGCACTACATCAA-3′ (SEQ ID NO: 68), 5′-GGCAGCCACCAGCAGGTACACTCTG-3′ (SEQ ID NO: 69), and 5′-CTGCTCAACAGGCTTGTTTGGATAT-3′ (SEQ ID NO: 70), 1 μg/head; GeneWorld Ltd.) or 10×PBS-diluted atelocollagen (Koken Co.) premixed with PBS was injected into the peritoneal cavities of the mice. The groups of mice treated as described above were named the “versican siRNA group” (n=6) and “control group” (n=4). The weight and the disease activity index (DAI) score were recorded during seven days of 3% DSS water feeding (Kihara, M., Gut. 2003, 52, 713-9). The evaluation criteria for DAI are shown below.

TABLE 1

| Index | Weight loss | Stool hardness | Bloody stool |
|---|---|---|---|
| 0 | Unaltered | Normal | Normal |
| 1 | 1-5% | | Occult blood (+) |
| 2 | 5-10% | Loose stool | Occult blood (++) |
| 3 | 10-20% | | Occult blood (+++) |
| 4 | >20% | Diarrhea | Bleeding |

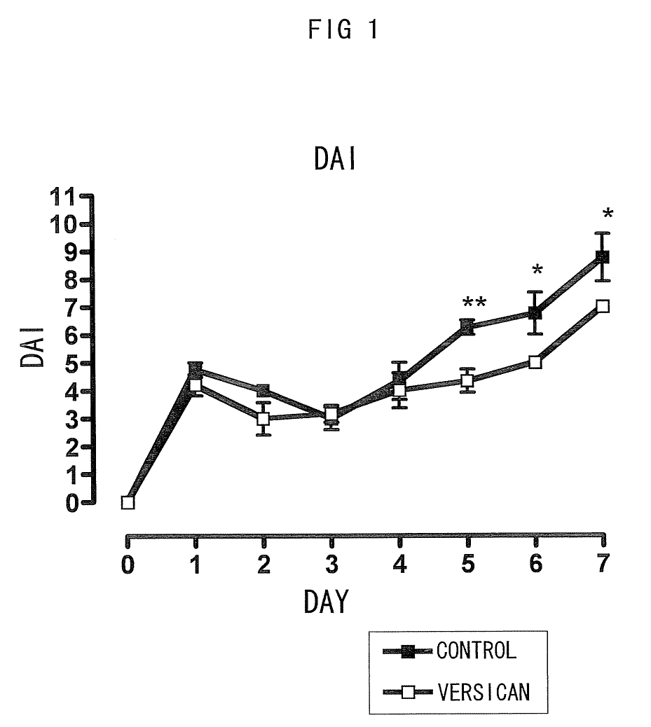

The weight and percent weight loss of each mouse were recorded, taking the weight on the first day of DSS water feeding (day 0) as 1. The result showed that there was no significant difference between the versican siRNA-treated group and the control group. In addition, the DAI for each mouse was recorded. The result showed that the DAI in the versican siRNA-administered group was significantly lower from day 5 to day 7 as compared with that of the control group (p<0.05; t test). This result showed that the suppression of versican gene expression by versican siRNA produces an inflammatory activity-suppressing effect (FIG. 1).

### Example 2

### Therapeutic Effects of Versican siRNA in Ulcerative Colitis Model Mice: Macroscopic Features

The ulcerative colitis model was prepared by allowing C57BL/6JcL mice (female, 6 weeks old; CLEA Japan Inc.) to freely drink high-concentration chlorine water comprising 3% dextran sulfate sodium (DSS; Wako Pure Chemical Industries Ltd.) for seven days. At the same time when the mice were feed with 3% DSS water, 200 μl of versican siRNA (1 μg/head; GeneWorld Ltd.) or 10×PBS-diluted atelocollagen (Koken Co.) premixed with PBS was injected into the peritoneal cavities of the mice. The groups of mice treated as described above were named the “versican siRNA-treated group” (n=6) and “control group” (n=4). The mice were grown for eight days and receiving 3% DSS water. Then, the mice in each group were sacrificed, and their large intestines were collected and the lengths were determined.

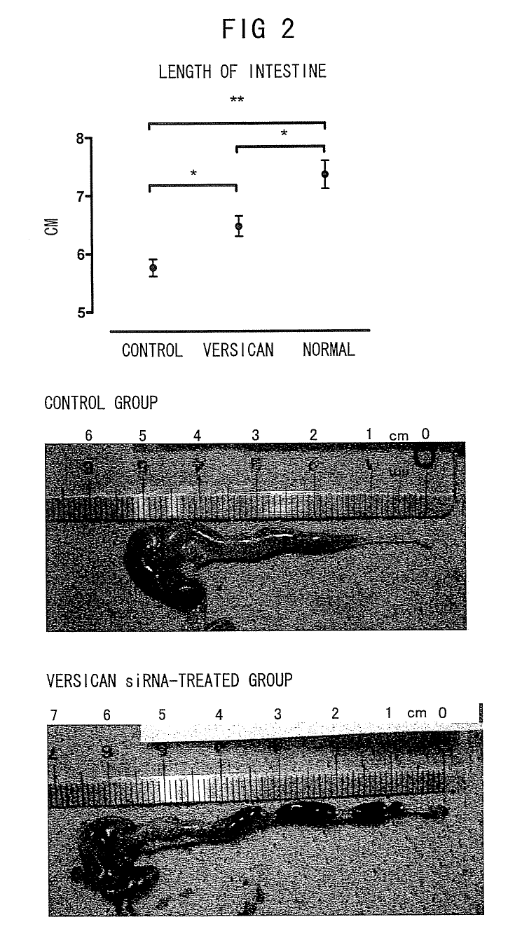

The result showed that the length of the intestines was significantly conserved in the versican siRNA-administered group as compared with the control group (p<0.05; t test). Hence, it can be assumed that atrophy of the large intestine due to fibrous degeneration is suppressed as a result of suppressed expression of the versican gene (FIG. 2).

### Example 3

### siRNA Suppression of Versican Expression in Ulcerative Colitis Model Mice

In this Example, the effect of administering versican siRNA in suppressing versican expression was evaluated by PCR using a typical mouse model of ulcerative colitis, the dextran sulfate sodium-induced ulcerative colitis mouse model.

The ulcerative colitis model was prepared by allowing C57BL/6JcL mice (female, 6 weeks old; CLEA Japan Inc.) to freely drink high-concentration chlorine water comprising 3% dextran sulfate sodium (DSS; Wako Pure Chemical Industries Ltd.) for seven days. At the same time when the mice were fed with 3% DSS water, 200 μl of versican siRNA (1 μg/head; GeneWorld Ltd.) or 10×PBS-diluted atelocollagen (Koken Co.) premixed with PBS was injected into the peritoneal cavities of the mice. The groups of mice treated as described above were named the “versican siRNA group” and “control group”. The mice were grown for eight days and receiving 3% DSS water. Then, the mice in each group were sacrificed, and their large intestines were collected and the lengths were determined.

After measuring the lengths of the large intestines collected, a portion of the intestines was placed in 1.5-ml tubes and frozen with liquid nitrogen. 1 ml of RNA-Bee (Tel-Test Inc.) was added to every 50 mg of tissues to produce a homogenized suspension. Then, 200 μl of chloroform (Sigma Aldrich Japan) was added to each suspension. After mixing gently, the suspensions were cooled on ice for about five minutes, and then centrifuged using a centrifuge (Centrifuge 5417R, Eppendorf) at 12,000 rpm and 4° C. for 15 minutes. After centrifugation, 500 μl of the supernatants were transferred to different 1.5 ml-tubes, and an equal volume (500 μl) of isopropanol (Sigma Aldrich Japan) was added to each tube. After mixing, 1 μl of glycogen (Invitrogen) was added to the tubes, which where then cooled on ice for 15 minutes. After cooling on ice for 15 minutes, the mixtures were centrifuged at 12,000 rpm and 4° C. for 15 minutes. Then, the RNA precipitates were washed three times with 1000 μl of 75% ethanol (Sigma Aldrich Japan), air-dried for 30 minutes to one hour, and dissolved in 50 μl of Otsuka distilled water (Otsuka Pharmaceutical). The RNAs were then diluted 100 times with Otsuka distilled water and the concentrations of extracted RNA in the samples were determined using UV plates (Corning Costar) in a plate reader (PowerWave XS, BioTek Inc.).

The concentrations of the yielded RNA samples were adjusted to 500 ng/20 μl. The samples were then heated at 68° C. for three minutes in a block incubator (Astec), and then cooled on ice for ten minutes. After cooling, 80 μl of previously prepared RT PreMix solution [composition: 18.64 μl of 25 mM MgCl₂ (Invitrogen), 20 μl of 5× buffer (Invitrogen), 6.6 μl of 0.1 M DTT (Invitrogen), 10 μl of 10 mM dNTP mix (Invitrogen), 2 μl of RNase inhibitor (Invitrogen), 1.2 μl of MMLV reverse transcriptase (Invitrogen), 2 μl of random primer (Invitrogen), and 19.56 μl of sterile distilled water (Otsuka distilled water, Otsuka Pharmaceutical)] was added to the samples. The resulting mixtures were incubated in a block incubator at 42° C. for one hour and at 99° C. for five minutes, and then cooled on ice. cDNAs were prepared in 100 μl.

PCR was carried out using the synthesized cDNAs in the following composition: 1 μl of the cDNAs was combined with 2 μl of PCR buffer [composition: 166 mM (NH₄)₂SO₄ (Sigma Aldrich Japan), 670 mM Tris (pH8.8) (Invitrogen), 67 mM MgCl₂·6H₂O) (Sigma Aldrich Japan), and 100 mM 2-mercaptoethanol (Wako)], 0.8 μl of 25 mM dNTP mix (Invitrogen), 0.6 μl of DMSO (Sigma Aldrich Japan), 0.2 μl of primer (forward 5′-GACGACTGTCTTGGTGG-3′ (SEQ ID NO: 71), reverse 5′-ATATCCAAACAAGCCTG-3′ (SEQ ID NO: 72), GeneWorld), 15.7 μl of Otsuka distilled water, and 0.1 μl of Taq polymerase (Perkin Elmer). The resulting mixtures were reacted in an Authorized Thermal Cycler (Eppendorf) with 35 cycles of 94° C. for 45 seconds, 56° C. for 45 seconds, and 72° C. for 60 seconds. After the reaction, 2 μl of loading dye (Invitrogen) was added to the yielded PCR products. 1.5% of UltraPure Agarose (Invitrogen) gel was prepared, and the PCR products were electrophoresed using Mupid-2 plus (Advance) at 100 V for 20 minutes. After the electrophoresis, the gel was shaken for 20 to 30 minutes in a staining solution of ethidium bromide (Invitrogen) 10,000-times diluted with 1× LoTE [composition: 3 mM Tris-HCl (pH7.5) (Invitrogen) and 0.2 mM EDTA (pH7.5) (Sigma Aldrich Japan)]. The gel was then photographed using an Exilim (Casio) installed in I-Scope WD (Advance) to observe gene expression.

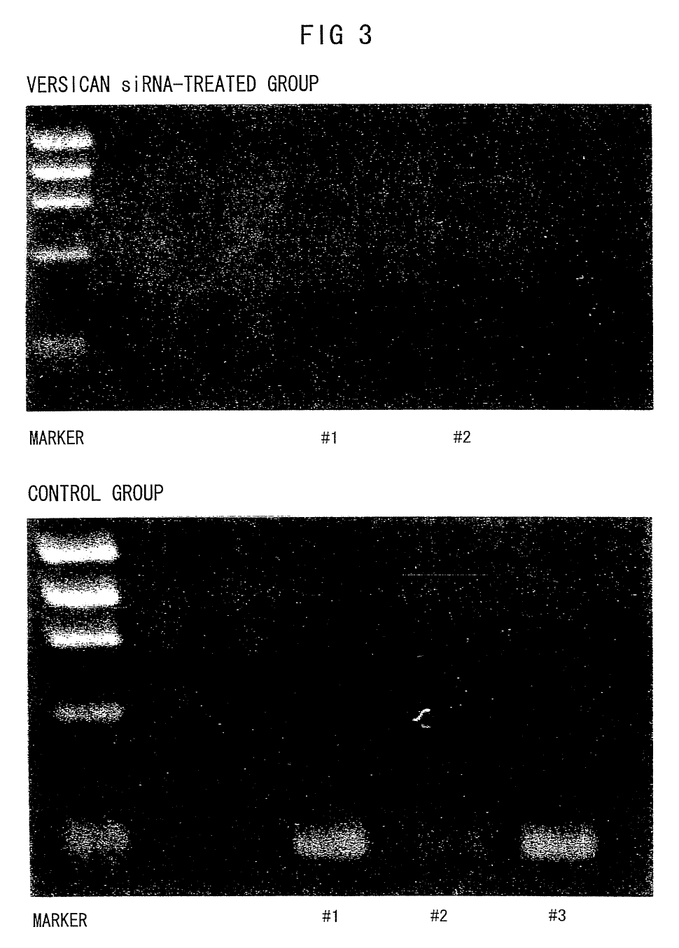

The result showed that the expression of versican mRNA was observed in the control group but not in the versican siRNA-treated group. This demonstrates that the expression of versican is suppressed by administering siRNA (FIG. 3).

### Example 4

### Therapeutic Effects of Versican siRNA in Ulcerative Colitis Model Mice: Histological Features

The ulcerative colitis model was prepared by allowing C57BL/6JcL mice (female, 6 weeks old; CLEA Japan Inc.) to freely drink high-concentration chlorine water comprising 3% dextran sulfate sodium (DSS; Wako Pure Chemical Industries Ltd.) for eight days. At the same time when the mice were fed with 3% DSS water, 200 μl of versican siRNA (1 μg/head; GeneWorld Ltd.) or 10×PBS-diluted atelocollagen (Koken Co.) premixed with PBS was injected into the peritoneal cavities of the mice. The groups of mice treated as described above were named the “versican siRNA group” and “control group”. The mice were grown for eight days and receiving 3% DSS water. Then, the mice in each group were sacrificed, and their large intestines were collected and the lengths were determined. After measuring the lengths of the large intestines collected, a portion of the intestines was embedded in OCT compound (Sakura Co.), an embedding medium for cryosectioning. Cryosections were prepared using liquid nitrogen and sliced into 6-μm sections using a cryostat (Microedge Inc.).

The obtained sections were fixed with acetone (Wako Pure Chemical Industries Ltd.) for 10 minutes, and then washed with phosphate buffer. An anti-F4/80 antibody (clone A3-1, rat monoclonal antibody, 2 μg/ml; CALTAG Laboratories), anti-ER-TR7 antibody (rat monoclonal antibody, 1 μg/ml; BMA), or anti-chondroitin sulfate antibody (clone CS-56, mouse monoclonal antibody, Seikagaku Co.) was added as the primary antibody, and the sections were allowed to react at room temperature for 1 hour. Then, the secondary antibody reaction was conducted using a peroxidase-labeled anti-rat IgG (at 1:200 dilution: used for the anti-F4/80 antibody and anti-ER-TR7 antibody) and Histofine Mouse Stain kit (Nichirei Bioscience; used for the anti-chondroitin sulfate antibody), and subsequently, DAB substrate (Nichirei Bioscience) was added for color development. Then, the nucleus was stained by Lillie-Mayer hematoxylin (Muto Pure Chemicals Co.). The samples were observed under a light microscope (Leica Microsystems). The antibody binding was visualized as brown signals.

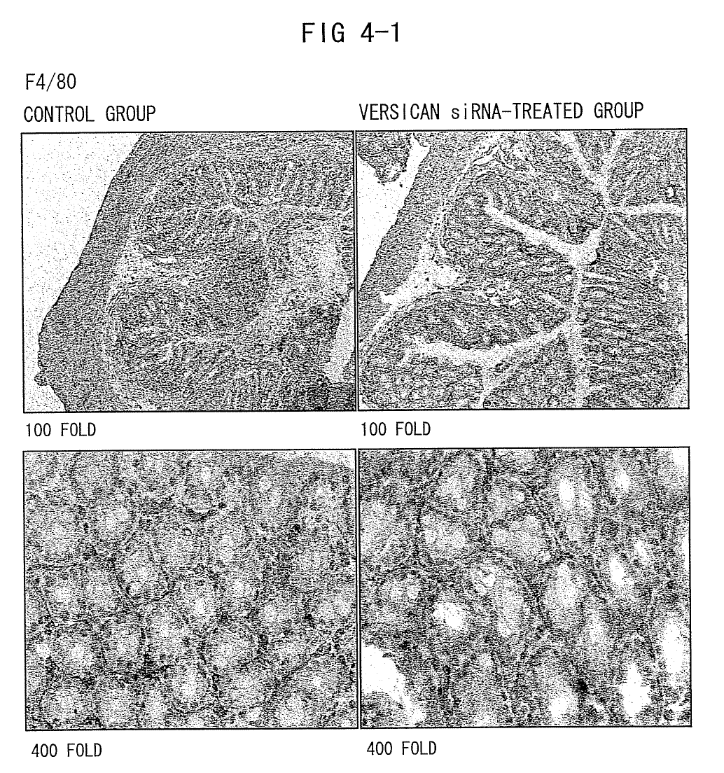

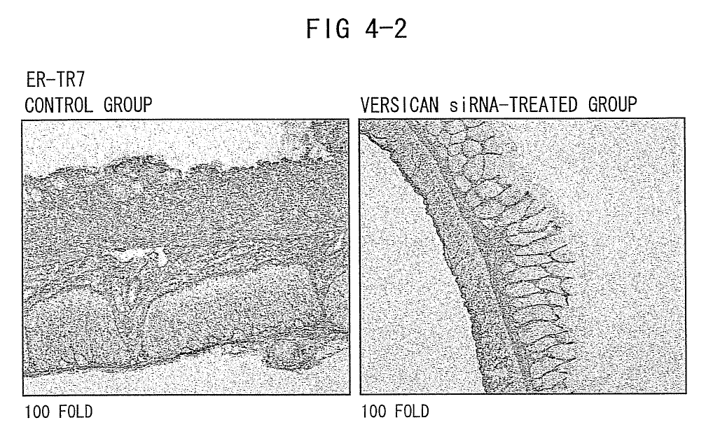

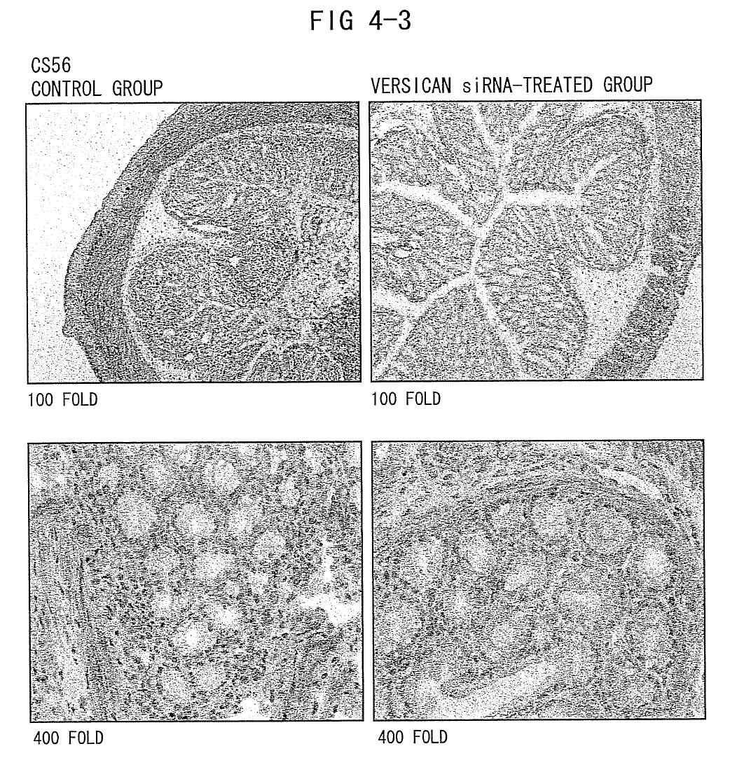

The result showed that as compared with the control group, the versican siRNA-treated group has an intact morphology of mucosal epithelia and goblet cells and no ulcerative lesion. There was little infiltration of macrophages (F4/80 positive cells) and reticular fibers/fibroblasts (ER-TR7 positive cells) in the lamina propria. Furthermore, the expression of chondroitin sulfate (CS56) was found to be suppressed in the versican siRNA-treated group as compared with the control group. These results demonstrate that the accumulation of chondroitin sulfate, infiltration of inflammatory cells, and fibrosis can be suppressed by administering versican siRNA to suppress the versican expression (FIG. 4).

### Example 5

### Therapeutic Effects of Administering ADAMTS-4 Peptide in Colitis Model Mice

Examples 1 to 4 describe siRNA treatment that suppresses versican itself, which is a CSPG. Meanwhile, this Example describes that a similar therapeutic effect can be obtained by administering a recombinant peptide of a protein called ADAMTS-4 (a disintegrin and metalloproteinase with thrombospondin motifs) which has an activity of cleaving the core protein of versican. It is meaningful that the concept of suppressing excess accumulation of CSPG (in this case, versican) could be confirmed by two different types of methods (CSPG gene silencing and a peptide that directly cleaves CSPG).

Mouse DSS colitis was induced by the same method as described in Example 1. The sequence of the ADAMTS-4 peptide used in the treatment was NH2-DRARSCAIVEDDGLQSAFTA-COOH (SEQ ID NO: 73) (a domain having the metalloproteinase activity of mouse ADAMTS-4; synthesized as a peptide of positions 336 to 355; GeneWorld Ltd.; 1 μg/head). Vehicle (PBS) was used in the control group. On day 8, the mice of both groups were sacrificed, and sections were prepared from large intestine tissues using the same method as described in Example 4 and were immunohistologically assessed.

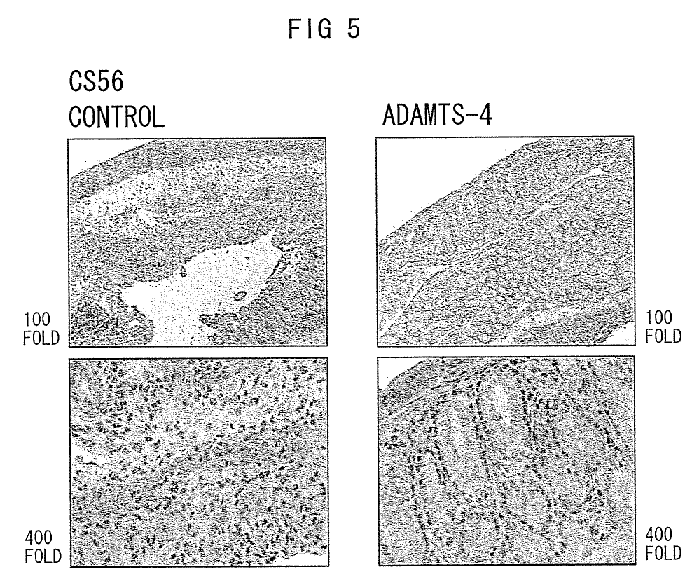

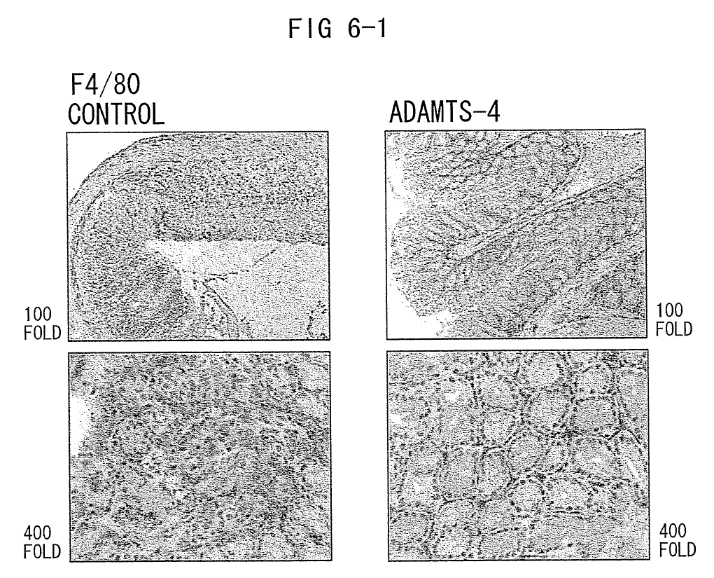

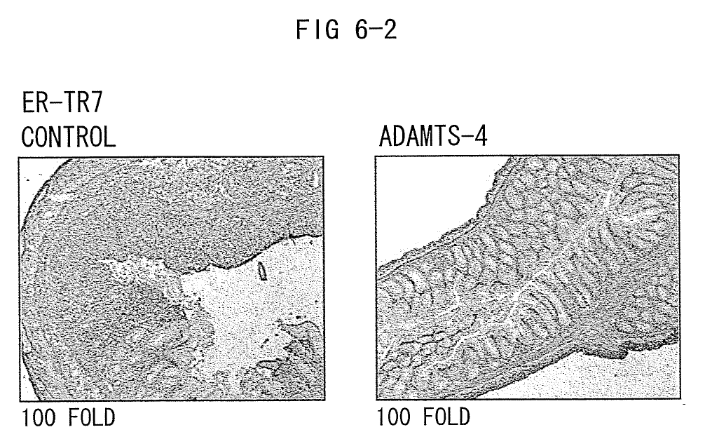

The result is shown in FIG. 5. Administration of the ADAMTS-4 metalloproteinase domain-equivalent peptide was found to reduce the deposition of CSPG in large intestine mucosa (CS56 staining). In addition, the infiltration of macrophages (F4/80) and fibroblasts (ER-TR7) into mucosa was significantly suppressed (FIG. 6). These histological findings suggest that administering the functional ADAMTS-4 peptide can suppress excess accumulation of CSPG and thereby inhibit the infiltration of inflammatory cells and fibrotic lesion.

### INDUSTRIAL APPLICABILITY

The ADAMTS-4 peptide and versican siRNA comprising the core site sequence of versican, which is one of the chondroitin sulfate proteoglycans, are used as an example to evaluate the effects of the accumulation of chondroitin sulfate proteoglycans (CSPG) in the present invention. They suppress the intestinal inflammation-associated accumulation of chondroitin sulfate proteoglycans and thus intestinal inflammation, and are therefore effective in treating or preventing inflammatory bowel diseases. When administered, the versican siRNA or ADAMTS-4 peptide suppresses versican expression and exerts an effect of suppressing the accumulation of chondroitin sulfate proteoglycans in areas surrounding the intestinal inflammation site. The intestinal inflammation-suppressing agents of the present invention are thus extremely useful in suppressing intestinal inflammation. Methods for treating or preventing inflammatory bowel diseases, which comprise administering an intestinal inflammation-suppressing agent of the present invention that has an effect of suppressing accumulation of chondroitin sulfate proteoglycans, can effectively improve lesions as a result of the novel action mechanism and pharmacological therapy. Thus, these methods are potential therapeutic methods which are useful and excellent for the improvement of patients' QOL and medical treatment.

All publications, patents, and patent applications cited herein are incorporated herein by reference in their entirety.

1. An intestinal inflammation-suppressing agent which comprises as an active ingredient a substance that inhibits the production or accumulation of a chondroitin sulfate proteoglycan.

2. The agent of claim 1, wherein the substance has an activity of promoting the degradation of a chondroitin sulfate proteoglycan.

3. The agent of claim 1, wherein the substance has an activity of inhibiting the synthesis of a chondroitin sulfate proteoglycan.

4. The agent of claim 1, wherein the substance has an activity of desulfating a chondroitin sulfate proteoglycan.

5. The agent of claim 1, wherein the substance has an activity of inhibiting the sulfation of a chondroitin sulfate proteoglycan.

6. The agent of claim 1, wherein the production or accumulation of a chondroitin sulfate proteoglycan is inhibited in the large intestine or the small intestine.

7. The agent of claim 1, which is used for treating or preventing an inflammatory bowel disease.

8. The agent of claim 7, wherein the inflammatory bowel disease is ulcerative colitis.

9. The agent of claim 7, wherein the inflammatory bowel disease is Crohn's disease.

10. A method of screening for an intestinal inflammation-suppressing agent, which comprises selecting from a test sample a substance with an activity of inhibiting the production or accumulation of a chondroitin sulfate proteoglycan.

11. The method of claim 10, which comprises the step of selecting a substance with the activity of any of:
(a) promoting the degradation of a chondroitin sulfate proteoglycan;
(b) inhibiting the synthesis of a chondroitin sulfate proteoglycan;
(c) desulfating a chondroitin sulfate proteoglycan; and
(d) inhibiting the sulfation of a chondroitin sulfate proteoglycan.

12. The method of claim 10, wherein the intestinal inflammation-suppressing agent is used for treating or preventing an inflammatory bowel disease.

13. A method of treating or preventing a disease accompanying intestinal inflammation, which comprises administering to a patient in need thereof a substance that inhibits the production or accumulation of a chondroitin sulfate proteoglycan.


SEQUENCE LISTING (74 sequences)

SEQ ID NO: 1 - DNA, 7382 bp, Mus musculus
  CDS (133)..(6531)
ccccccgcgcagagcatccctgctgcagcgcagcaagacccggggctggcagccccaggaatccctagctgcttcgcagggataaaggactgaagttcttggaggagcgagtccaactcttcaagctgaactatgaccactttactcttggtctttgtgactctgagggtcatcgctgcagtgatctcagaagaagttccagaccatgacaactcactgagcgtgagcatccctcaaccatccccattgaaggtcctcctagggtcttccctcaccatcccctgctacttcatcgaccccatgcatcctgtgaccactgccccctccactgcccccctcaccccaagaatcaagtggagccgtgtttccaaggaaaaggaggtggtactgctggtggccactgaaggacaggttcgagtcaacagcatctaccaagacaaggtgtcgctccccaactatccagccatccccagcgatgctaccttggagatccagaaccttcgctccaatgactctgggatctaccgctgtgaagtgatgcacggcatcgaggacagcgaagccaccctggaggtcatagtgaaaggtattgtgttccactacagagctatttccacacgctacaccctggactttgatcgagcacagcgggcttgcctacagaacagcgccatcatcgccaccccagaacaactgcaggctgcctatgaggatggcttccaccagtgcgatgcaggctggctggctgaccagacagtcagataccccatccacacgccccgggaaggttgctatggtgacaaggacgagttccctggagtgagaacctacggaatccgggacaccaatgagacctatgatgtgtactgcttcgctgaggagatggagggtgaggtcttttatgcgacatccccagagaaattcaccttccaggaggcagccaacgagtgccggaggctgggggcacgcttggccaccacaggccagctctacctggcctggcagggcggtatggacatgtgcagcgctggctggctggcggaccgcagcgtgcgctaccccatctccaaggctcggcccaactgcgggggcaacctcctgggtgtaaggactgtctatctacacgccaaccagacaggctatcctgatccctcatcccgatatgacgccatctgttacacaggtgaagattttgtagacatcccagaaaacttcttcggggtgggtggtgaagacgacatcaccatccagacggtgacctggccagacctggagctgcccttgccccgtaatgtcacagagggagaggccctgggcagcgtgatcctcacggcaaagcccatctttgacctgtcccccactatctcagagccgggggaggccctcacacttgcccctgaagtggggagcacagccttcccagaggctgaggagagaactggagaagccaccagaccctggggctttcctgcagaagtcacacgtgggccggactctgccactgcctttgccagtgaggacctggtagtgcgagtgaccatctctccaggtgcagctgaagtccctggtcagccccacttgccagggggagttgtattccactatcgcccaggctccaccagatactctctgacatttgaggaggcacagcaggcctgcatgcacaccggggccatcattgcctctcctgagcagctccaagctgcctatgaggcaggctatgagcagtgtgatgctggctggctgcaggaccagaccgtcagataccccattgtgagcccacgaaccccatgtgtgggtgacaaagacagcagcccaggagtcaggacctaccgcgtgcgcccatcatcagaaacctatgatgtctactgctacgtggacaagcttgagggggaagtgttcttcgccacacgcctcgagcagttcaccttccaggaagcgcgggccttctgtgcggctcaaaatgccaccctggcctccaccggccagctctatgctgcctggagccagggtctggacaagtgctatgctggctggctggcagatggcaccctccgataccccatcatcacccctcggcctgcctgtggtggggacaaacctggcgtgagaactgtctacctctaccccaaccaaaccggcctccctgacccactgtcaaagcaccatgccttctgcttccgaggtgtgtcagtggcgccctctccaggagaagaagagggtagtacacccacatcaccctctgacatagaggactggatcgtcactcaggtggggcctggtgtggatgctgtccccttggagccaaagacaacagaagtgccatatttcaccactgagccaagaaaacagactgaatgggagccagcctacaccccagtgggcacatccccacagccagggatccctcccacatggcttcccaccctcccagcagcagaggaacacacagaaagcccctctgcctctgaagagccctctgcctcagcagtcccttccacctcagaggagccatacacatcttcatttgcagtgccgagcatgacagagctgccaggctctggggaggcgtcgggcgcacctgacctcagtggtgacttcacaggcagtggagatgcttcaggacgccttgactccagtgggcagccttcagggggcattgaaagtggccttccctcaggtgaccttgactccagtggcctcagccccacagtgagctcaggcctgcctgtagaaagtggttctgcctcaggagatggagaagtcccctggtcccatactcccacagttggcaggttgccctctggaggtgagagccccgaaggctctgcctctgcctctggaacaggagaccttagtgggctgccttcaggaggagaaattacagaaacttctacttctggggcagaagaaaccagtggacttccttctggaggagacggtctagaaacttctacctctggagtagatgatgtcagtggaattcctactggaagagaaggtctagagacttctgcctctggagtagaggacctcagtggacttccttctggagaagaaggttcagaaacatctacctctggaatagaggacatcagtgtacttccaactggaggagaaagtctagaaacctctgcttctggagtgggagacttgagtggacttccctcaggaggagaaagtctagaaacatctgcttcaggtgcagaggatgtcactcagcttcctactgaaagaggaggtctagagacttctgcctctggagtagaagacatcactgttcttcctactggaagagaaagtctagaaacttctgcctctggagtagaggatgtcagtggacttccttctggaagagaaggtctagagacttctgcctctggaatagaggacattagtgtgtttcctactgaagcagaaggtctggacacttctgcctctgggggatatgttagtgggattccttctggaggagatggtacagaaacctctgcttctggagtagaggatgtgagtggtcttccatctggaggagagggtctagaaacttctgcctctggagtggaagatcttggtccttctactagagatagtctagagacatctgcttcaggagtagatgttactgggtttccttctggaagaggggacccagagacctctgtttctggggtaggtgatgacttcagtggacttccttctggaaaagaaggcctggagacctcagcttctggagctgaggacctcagtggcttgccctctggaaaagaagacttggtagggtctgcttctggggccttggactttggcaaactacctcctggaactctaggaagtggtcaaactccagaagtaaatggctttccctctggatttagtggtgagtattctggagcagacattggaagtggcccatcctctggcctgcctgactttagtggacttccatctggctttccaactgtctcccttgtggacagtaccttagtggaagtgatcacagccaccacttccagtgaactggaaggaagggggaccattggcatcagtggttcaggagaagtatcagggctgcccctgggtgaattggacagtagtgcggacattagtggtctcccttcaggaactgaactcagtggccaagcatctggatctcctgatagcagtggagaaacatctggattttttgatgttagtggacagccatttgggtcttctggcgtcagcgaggaaacatctgggattcctgaaatcagtgggcagccatcagggactcctgacaccactgcgacatctggagtgactgagcttaatgaactgtcctctggacaaccagatgtcagtggagatgggtctggaattctctttggcagtggacagtcctctggtataacatctgtgagtggagaaacctctgggatttctgatctcagtgggcagccctcagggttcccagtgttcagtggaacagcaaccagaacccctgacctggcttctggcaccataagtggcagtggagagtcttctggcattacatttgtggacaccagttttgttgaagtgacccctaccacatttagggaagaagaagggttaggatctgtggaactcagtggctttccttctggggagacggaactgtctggcacatctgggacggtggacgtcagtgaacaatcttctggagcaattgattccagtggactcacatcccccactccagagttcagtggcctcccaagtggagtagctgaggtcagtggtgaattctctggagttgagactgggagcagcttgccctcaggagcatttgatggcagtggacttgtctcaggtttccccactgtgtctcttgtagacagaactttggtggaatctataactcaggctcctactgctcaagaagctggagaaggaccttcgggcattttggaattcagtggtgcccattctgggacaccagacatatctggggagctttctgggtctctggacctaagcacattgcagtctgggcagatggaaaccagcacggagacaccaagctctccatattttagtggagacttttccagcaccactgatgtaagtggagaatccatagctgccacaactggcagtggggaaagctctgggcttccggaagttactttaaacacctcagagttagtggagggtgtgactgaacccactgtttcccaggaacttggccatggtccttctatgacatacatctcccggctctctgaggccagtggggacgcctcagcatccggggaccttggtggcgctgtaacaaacttcccagggtctggagtagaagcttcagtcccagaagccagcagtgacctgtctgcttaccctgaggctggtgtgggagtgtctgctgcccctgaggccagcagtaaactgtctgagttcccagatctgcatggaatcacttctgccttccatgaaacagatctggaaatgacaaccccaagcacagaggtaaacagcaacccatggacctttcaggaaggcaccagggagggatcggccgctccggaagtgagtggagaatctagcactacctccgacatagacacaggcacttcaggggtgccttctgccacacccatggcttctggagacaggactgaaatcagcggagaatggtctgatcacacctcagaggtgaatgttgccatcagcagcaccatcacagagtccgagtgggcccagcctacccggtaccctacagagacacttcaagaaatcgaatccccaaatccctcatactcaggagaggagacccagacagcagaaacaaccatgtccctgacagatgcccccaccctctcttcttcagaagggtcaggggagacggagtcaaccgttgcagaccaggagcaatgtgaggaggggtggaccaagttccagggtcactgttaccgccactttcctgaccgagagacctgggtggatgcggagagacggtgtcgggagcagcagtcacatctgagcagcattgtcactcctgaggaacaggagttcgtcaacaaaaatgctcaagactaccagtggatcggtctgaatgacaggactatcgaaggggacttccgctggtctgacggacactctctgcaatttgagaagtggcgtccaaaccagcctgacaacttctttgccaccggagaggactgtgtggtgatgatctggcatgagagaggcgaatggaacgacgtcccctgcaattaccagctgcccttcacgtgtaaaaagggcaccgtggcctgtggagaccccccagtggtggagcatgctagaaccctcgggcagaagaaagatcgctacgagatcagctccctggtgcggtaccagtgcactgagggctttgtccagcgccacgtgcccaccatccggtgccagcccagcgggcactgggaagagcctcgaatcacctgcacagaccccaacacctacaagcacaggctacagaagcggacgatgagacccacacggaggagccgccccagcatggcccactgagaggagcttccataatgtgcccaggatgctgagcccagcggccagccaggctgaccgtgcatcccacccacatggtgtcttcttgtcgctttttgtcatataaggaatccattaaagaaggaaaaaaaacccacattgtgtgtgccccactccccgcatcacccaaactgcatctaatttatccccttctcccccccccccaaaaaaaaaaagaaaagaaatgcccaagcaagtcactgtaaccccgtggtacctcagctttgagacttttttgatttccccacccgtgccctttcccagggaccagtgcaggcacagggtgagaaggtaaggggttaaattaaataaagaagattcttttttgttgttgtttcctgactttgcccaagaacagtacaatggttggttacttcgcctccagggagagctaggggcccagaggaggagctgaaagaagacaggaccaggaagggaggagtagggcggtcacagagcttggaggactcagaagaggcaaagtacagcttagtgtgttggacaaaaggaattgaggtctgtgccatctgtgagggaaggacctggggcagggacaggctggctgaagagaagcagagccctctcctagggacctttcatctgggtcaaccaacctagcaggtgtcatttctcggctggactgggtaggggcacttccttcccaggggtctccgccgtccccttccctctccccaccccgggacggtgcaggcaccagtgttccgtgcacctatttatatttttgaaaactaaagattatgataattataataataaagacattggaagagatctaaaaaaaaaaaaaaaaaaaa

SEQ ID NO: 2 - protein, 2132 aa, Mus musculus
MTTLLLVFVTLRVIAAVISEEVPDHDNSLSVSIPQPSPLKVLLGSSLTIPCYFIDPMHPVTTAPSTAPLTPRIKWSRVSKEKEVVLLVATEGQVRVNSIYQDKVSLPNYPAIPSDATLEIQNLRSNDSGIYRCEVMHGIEDSEATLEVIVKGIVFHYRAISTRYTLDFDRAQRACLQNSAIIATPEQLQAAYEDGFHQCDAGWLADQTVRYPIHTPREGCYGDKDEFPGVRTYGIRDTNETYDVYCFAEEMEGEVFYATSPEKFTFQEAANECRRLGARLATTGQLYLAWQGGMDMCSAGWLADRSVRYPISKARPNCGGNLLGVRTVYLHANQTGYPDPSSRYDAICYTGEDFVDIPENFFGVGGEDDITIQTVTWPDLELPLPRNVTEGEALGSVILTAKPIFDLSPTISEPGEALTLAPEVGSTAFPEAEERTGEATRPWGFPAEVTRGPDSATAFASEDLVVRVTISPGAAEVPGQPHLPGGVVFHYRPGSTRYSLTFEEAQQACMHTGAIIASPEQLQAAYEAGYEQCDAGWLQDQTVRYPIVSPRTPCVGDKDSSPGVRTYRVRPSSETYDVYCYVDKLEGEVFFATRLEQFTFQEARAFCAAQNATLASTGQLYAAWSQGLDKCYAGWLADGTLRYPIITPRPACGGDKPGVRTVYLYPNQTGLPDPLSKHHAFCFRGVSVAPSPGEEEGSTPTSPSDIEDWIVTQVGPGVDAVPLEPKTTEVPYFTTEPRKQTEWEPAYTPVGTSPQPGIPPTWLPTLPAAEEHTESPSASEEPSASAVPSTSEEPYTSSFAVPSMTELPGSGEASGAPDLSGDFTGSGDASGRLDSSGQPSGGIESGLPSGDLDSSGLSPTVSSGLPVESGSASGDGEVPWSHTPTVGRLPSGGESPEGSASASGTGDLSGLPSGGEITETSTSGAEETSGLPSGGDGLETSTSGVDDVSGIPTGREGLETSASGVEDLSGLPSGEEGSETSTSGIEDISVLPTGGESLETSASGVGDLSGLPSGGESLETSASGAEDVTQLPTERGGLETSASGVEDITVLPTGRESLETSASGVEDVSGLPSGREGLETSASGIEDISVFPTEAEGLDTSASGGYVSGIPSGGDGTETSASGVEDVSGLPSGGEGLETSASGVEDLGPSTRDSLETSASGVDVTGFPSGRGDPETSVSGVGDDFSGLPSGKEGLETSASGAEDLSGLPSGKEDLVGSASGALDFGKLPPGTLGSGQTPEVNGFPSGFSGEYSGADIGSGPSSGLPDFSGLPSGFPTVSLVDSTLVEVITATTSSELEGRGTIGISGSGEVSGLPLGELDSSADISGLPSGTELSGQASGSPDSSGETSGFFDVSGQPFGSSGVSEETSGIPEISGQPSGTPDTTATSGVTELNELSSGQPDVSGDGSGILFGSGQSSGITSVSGETSGISDLSGQPSGFPVFSGTATRTPDLASGTISGSGESSGITFVDTSFVEVTPTTFREEEGLGSVELSGFPSGETELSGTSGTVDVSEQSSGAIDSSGLTSPTPEFSGLPSGVAEVSGEFSGVETGSSLPSGAFDGSGLVSGFPTVSLVDRTLVESITQAPTAQEAGEGPSGILEFSGAHSGTPDISGELSGSLDLSTLQSGQMETSTETPSSPYFSGDFSSTTDVSGESIAATTGSGESSGLPEVTLNTSELVEGVTEPTVSQELGHGPSMTYISRLSEASGDASASGDLGGAVTNFPGSGVEASVPEASSDLSAYPEAGVGVSAAPEASSKLSEFPDLHGITSAFHETDLEMTTPSTEVNSNPWTFQEGTREGSAAPEVSGESSTTSDIDTGTSGVPSATPMASGDRTEISGEWSDHTSEVNVAISSTITESEWAQPTRYPTETLQEIESPNPSYSGEETQTAETTMSLTDAPTLSSSEGSGETESTVADQEQCEEGWTKFQGHCYRHFPDRETWVDAERRCREQQSHLSSIVTPEEQEFVNKNAQDYQWIGLNDRTIEGDFRWSDGHSLQFEKWRPNQPDNFFATGEDCVVMIWHERGEWNDVPCNYQLPFTCKKGTVACGDPPVVEHARTLGQKKDRYEISSLVRYQCTEGFVQRHVPTIRCQPSGHWEEPRITCTDPNTYKHRLQKRTMRPTRRSRPSMAH

SEQ ID NO: 3 - DNA, 2291 bp, Mus musculus
  CDS (257)..(1363)
aagaggtaatccagaactcaaggagcgcgggcgcagcagctgtgagcattaggtgctgtggccaagcgctgggacaaggatctgccgctaccagcgcatcctctctcctctccagctctgtcccgcactcgctgcacccttccccaggccaccgcgcttcctatgtgatcttccggggcaacgcggagccctttctcacagctcagcagtgaatttccaccccgaaaactgcagtaagccgcctttcaaggacaagatgttgataaatatgaaaggcatcttatggatgtgctcaaccttattactaacccatgcactacatcaagccaaaatggaaaccagcccacctgttaaaggctctctgtctggaaaagtggtcctaccttgtcatctttcaaccttacctaccttaccacccaattacaacacgagtgaatttctcagaatcaaatggtctaagatggaagtggacaaaaatggaaaagatataaaggagacgactgtcttggtggcccagaacggaaatatcaagattggtcaggactacaaggggcgagtgtccgtgcctacacatcccgatgatgtaggtgatgcttccctcaccatggtcaaactccgggctagtgatgcaggcgtctaccgatgtgatgtcatgtatgggattgaagacactcaggacaccatgtcactggctgtggatggtgttgtgtttcactacagggcagccaccagcaggtacactctgaactttgccgctgctcaacaggcttgtttggatatcggggcggtcatagcaagcccagagcagctgtttgccgcctatgaggatggatttgagcagtgtgatgcaggatggctgtctgatcaaactgtcagatatcccatacgggctccccgagagggctgttacggagacatgatggggaaggaaggggttcggacctatggattccgctctccccaggaaacctatgatgtgtattgttatgtggatcatctggatggcgatgtgttccacatcactgctcccagtaagttcaccttcgaggaggccgaagcagagtgtacaagcagggatgcgaggctggcgactgttggagaacttcaggcagcttggagaaatggctttggccaatgcgattacggctggctgtcggatgccagcgtgcggcaccctgtgactgtggccagggcccagtgtggaggaggtctacttggggtgagaaccctgtatcgttttgagaaccagacatgcttccctctccctgatagcagatttgatgcctactgctttaaacgtaagtgtttgatatctcttttaaaaatacagattaaaaagctgcagcaaatatatacccactgaggttccagtggatgtcgggatggattctgtattctgtactaagtgcatggaggacctacagtgcagtgacacaaacagacttaataccaccattatggttaacacagtagggagtttggcaagtgggattttttttgggaggtggggagataacaacgtcagagtggggtcttaactgcaccccagaattccaaaaatcacagaacacaccctgaattgaaatttcttattaattgaagcctttcaatgcaatttcactcttaatataacaatgttaagacaacagcagctgagttaaacctttgaatcctgttttattttttgatatgattcttattataaaaaggagacaatttaagcttaagtcattgcaggtgagtatttgtgctgaaattaataatgattgtgttcaattcctacctaaaaatgcatcatactatcaaataatacgattcacatatctcattttgtttcaagtaatagaataggcgccttttggcgattaatcttgctttgaattaattaaagtacaatacagcctccttgcatgaattggctatgctcagttagatttgccttgtaaaattaattttaggtagcattcattacatatatatatatgtatatatatatattagacattcttaaattattttaaaatattagtcacatacagttttatagttgtagagtgcttaactaaactaataactagtcaatattttattgtaatatgtactttaggatcggtaagatgttagctaacaaatgcctccatactttagcaacagaggccaagtctgacatcgagtatcagaacaatgaaaagccaatgtaacacatgaagtcttttaaaccagctttctaattccttttgttagatcaatgggtaattaaaatagcttgaaaatgtaaaaaaaaaaaaaaa

SEQ ID NO: 4 - protein, 368 aa, Mus musculus
MLINMKGILWMCSTLLLTHALHQAKMETSPPVKGSLSGKVVLPCHLSTLPTLPPNYNTSEFLRIKWSKMEVDKNGKDIKETTVLVAQNGNIKIGQDYKGRVSVPTHPDDVGDASLTMVKLRASDAGVYRCDVMYGIEDTQDTMSLAVDGVVFHYRAATSRYTLNFAAAQQACLDIGAVIASPEQLFAAYEDGFEQCDAGWLSDQTVRYPIRAPREGCYGDMMGKEGVRTYGFRSPQETYDVYCYVDHLDGDVFHITAPSKFTFEEAEAECTSRDARLATVGELQAAWRNGFGQCDYGWLSDASVRHPVTVARAQCGGGLLGVRTLYRFENQTCFPLPDSRFDAYCFKRKCLISLLKIQIKKLQQIYTH

SEQ ID NO: 5 - DNA, 2583 bp, Mus musculus
  CDS (162)..(2339)
cggcagtggctggcaagaaacaattctgcaaaaataatcatacccagcctggcaattgtctgctcctcggtccattgctccgccgccgtccacagtcgcttgcaaggggaaggcactgaatttaccgcggccagaacatccctcccagccggcagtttacaatgctgcgaactaaggatctcatctggactttgtttttcctgggaactgcagtttccctgcaggtagatattgttcccagccaaggagaaatcagcgttggagagtccaaattcttcctgtgtcaagtggcaggagatgccaaagataaggacatctcctggttctcccccaatggggagaagctgagcccaaaccagcagcggatctcagtggtgtggaatgatgacgactcctctaccctcaccatctacaacgccaacatcgacgatgccggcatatacaagtgtgtggtcaccgctgaggacggcacccagtctgaggccactgtcaacgtgaagatcttccagaagctcatgttcaagaatgcaccaaccccacaggagtttaaggaaggggaggatgctgtgattgtctgtgatgtggtcagctccctgcctccaaccatcatctggaaacacaaaggccgagatgtcattctgaaaaaagacgtccggttcatagtcctgtccaacaactacctgcagatcaggggcatcaagaaaacagatgagggtacttaccgctgtgagggcaggatcctggcccgcggggaaatcaacttcaaggacattcaggtcattgtgaatgtaccacccactgtccaggccagacagagcatcgtgaatgccactgccaacctgggccagtctgtcaccctggtgtgtgatgctgatggatttccagagcccaccatgagctggacaaaggatggggaacccatagagaacgaggaggaagatgagagaagcagatcttcagtgagtgacagctccgaggtgaccatcaggaatgtggataaaaacgacgaggccgaatacgtctgcatcgcagagaacaaggctggcgagcaggatgcctccatccacctcaaggtctttgcaaagcccaaaatcacctatgtagagaatcagacggccatggaactagaggagcaagtcactctgacctgtgaagcctccggagaccccatcccgtccatcacgtggagaacatccacccggaacatcagcagtgaagaacaggatctggatgggcacatggtggttcgcagccatgctcgtgtgtcctccttgaccctgaagagcatccagtacagagatgctggagaatacatgtgcactgccagcaacaccatcggccaggactcccagtccatcgacctcgaatttcaatatgctcccaagctccagggccctgtggctgtctacacctgggaagggaaccaagtgaacatcacctgtgaggtctttgcctatcccagtgccacaatctcctggttccgagatggtcagttgctgccaagctccaactacagcaatatcaagatctacaacaccccatctgctagctatctggaggtgaccccagattcagaaaatgactttggaaactacaactgtacagcagtgaaccgtattggacaggagtccttggaattcatccttgttcaagcagacacaccgtcttctccatccattgaccgggtggaaccatactccagcacagcgcaggtgcagtttgatgagccagaggccacagggggcgtgcccattctcaagtacaaggctgagtggaagtcgctgggcgaagaatcctggcatttcacgtggtatgatgccaaagaagccaacatggagggcattgtcaccatcatgggcctgaaacctgagacgacgtactcggatcgactggcggccctcaacggcaaggggctgggcgagatcatgcagccatctgagtccaagacacagccagttccggaactcagtgcacccaagctggaagggcagatgggagaggacgggaactccatcaaggtgaacctgatcaagcaggatgacggaggctcccccatcagacactatctggtcaagtacagagcgctcgcctctgagtggaaaccggaaatcaggctcccatccggcagtcaccacgtcatgctcaagtccctggactggaacgcagagtatgaagtctatgtggtagctgaaaaccagcaaggaaaatccaaggcagctcactttgtgttcaggacctcagcccagcccacggccatcccagccaccctgggcggaagctccacctcctacaccttggtctcattgcttttctctgcggtgactcttcttctgctctgatagaaacttgaacatgcatgtgtgcgagctctcatgtgcgcatgcgcacacgcacacacacgcacacgcacacgcacacgcacgcacacgcacacacacacacacacagacacacacacgcacacacacacgcacatgcacacgcgcacacacgcacacgcacacgcacacgcacgcacacacacacacacacacaccattgtatttgattaaaaagcccagttcctataaaaaaaaaaaaaaa

SEQ ID NO: 6 - protein, 725 aa, Mus musculus
MLRTKDLIWTLFFLGTAVSLQVDIVPSQGEISVGESKFFLCQVAGDAKDKDISWFSPNGEKLSPNQQRISVVWNDDDSSTLTIYNANIDDAGIYKCVVTAEDGTQSEATVNVKIFQKLMFKNAPTPQEFKEGEDAVIVCDVVSSLPPTIIWKHKGRDVILKKDVRFIVLSNNYLQIRGIKKTDEGTYRCEGRILARGEINFKDIQVIVNVPPTVQARQSIVNATANLGQSVTLVCDADGFPEPTMSWTKDGEPIENEEEDERSRSSVSDSSEVTIRNVDKNDEAEYVCIAENKAGEQDASIHLKVFAKPKITYVENQTAMELEEQVTLTCEASGDPIPSITWRTSTRNISSEEQDLDGHMVVRSHARVSSLTLKSIQYRDAGEYMCTASNTIGQDSQSIDLEFQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDRVEPYSSTAQVQFDEPEATGGVPILKYKAEWKSLGEESWHFTWYDAKEANMEGIVTIMGLKPETTYSDRLAALNGKGLGEIMQPSESKTQPVPELSAPKLEGQMGEDGNSIKVNLIKQDDGGSPIRHYLVKYRALASEWKPEIRLPSGSHHVMLKSLDWNAEYEVYVVAENQQGKSKAAHFVFRTSAQPTAIPATLGGSSTSYTLVSLLFSAVTLLLL

SEQ ID NO: 7 - DNA, 3153 bp, Mus musculus
  CDS (151)..(2802)
  misc_feature (3043)..(3043): n is a, c, g, or t
gaggctcccggcgagctggcgcccctgtctgggtcccgcgcgcccggccctgctcgcgcccgcgcatcgcgccgcagtctcggtctgcggctgcgggacgtgacggcgtgcgcggaggggacctcgcaagttcttccatcagtgtgcagaatgataccactgcttctgtccctgctggccgctctggtcctgacccaagcccctgccgccctcgctgatgacctgaaagaagacagctcggaggatcgagccttccgcgtgcgcatcggtgccgcgcagctgcggggcgtgctgggcggtgccctggccatcccatgccacgtccaccacctgcggccgccgcgcagccgccgggccgcgccgggttttccccgggtcaagtggaccttcctgtccggggaccgggaggtagaggttctggtggctcgcgggctgcgcgtcaaggtaaacgaagcctaccggttccgcgtggcgctgcctgcctaccccgcatcgctcacggatgtgtctctagtattgagcgaactgcggcccaatgattccggggtctatcgctgcgaggtccagcacggtatcgacgacagcagtgatgctgtggaggtcaaggtcaaaggggtcgtcttcctctacagagagggctctgcgcgctatgctttctccttcgctggagcccaggaagcctgcgctcgcataggagcccgaatcgccaccccggagcagctctatgctgcctacctcggcggctatgagcagtgtgatgcaggctggctgtccgaccaaactgtgaggtaccccatccagaacccacgagaggcctgctctggagacatggatggctatcctggcgtgcggaactacggagtggtgggtcctgatgatctctatgatgtctactgttatgccgaagacctaaatggagaactgttcctaggcgcccctcccagcaagctgacatgggaggaggctcgggactactgtctggaacgtggtgcacagatcgctagcacaggccagctgtacgcagcctggaatggtggcctggacagatgtagccctggctggctggctgatggcagcgtgcgctatcccatcatcacacccagccaacgctgtgggggcggcctgccaggagtcaagaccctcttcctctttcccaaccagactggcttccccagcaagcagaaccgcttcaatgtctactgcttccgagactctgcccatccctctgcttcctctgaggcctctagcccagcctcagatggacttgaggccattgtcacagtgacagaaaagctggaggaactgcagctgcctcaggaagcgatggagagcgagtctcgtggggccatctactccatccccatctcagaagatgggggaggaggaagctccaccccagaagacccagcagaggcccccaggactccgctagaatcggaaacccaatccattgcaccacctaccgagtcctcagaagaggaaggcgtagccctggaggaagaagaaagattcaaagacttggaggctctggaggaagagaaggagcaggaggacctgtgggtgtggcccagagagctcagcagccctctccctactggctcagaaacagagcattcactctcccaggtgtccccaccagcccaggcagttctacagctggatgcgtcaccttctcctgggcctccaaggttccgtggaccgcctgcagagactttgctccccccgagggagtggagcgccacatctactcctggtggggcaagagaagtagggggggaaactgggagccctgagctctctggggttcctcgagagagcgaggaggcagggagctccagcttggaggatggcccttccctacttccagctacatgggcccctgtgggtcccagggagctggagaccccctcagaagagaagtctggaagaactgtcctggcaggcacctcagtgcaggcccagccagtgctgcccaccgacagtgccagccacggtggagtggctgtggctccctcatcaggtgactgtatccccagcccctgccacaatggtgggacatgcttggaggagaaggagggtttccgctgcctatgtttgccaggctatgggggggacctgtgcgatgttggccttcatttctgcagccctggctgggaggccttccagggagcctgctacaagcacttttccacacgaaggagttgggaggaggcagaaagtcagtgccgagcgctaggtgctcatctgaccagcatctgcacccctgaggagcaagactttgtcaatgatcgataccgggagtaccagtggattgggctcaatgacaggaccatcgagggtgacttcttgtggtcagatggtgcccctctgctctatgaaaactggaaccctgggcagcctgacagctacttcctgtctggggagaactgtgtggtcatggtgtggcatgaccagggacagtggagtgatgtgccctgcaactaccatctatcctacacctgcaagatggggcttgtgtcctgtgggcctccaccacagctacccctggctcaaatatttggtcgccctcggctgcgctacgcggtggatactgtgcttcgatatcgatgccgagacgggctggctcagcgcaacctgccgttgatccgctgccaggagaatgggctttgggaggcccctcagatttcctgtgtaccccggaggcctggccgtgctctgcgctccatggacgccccagaaggaccacggggacagctctcgaggcacaggaaggcaccgttgacaccgccctccagtctctagggagcctggaagactgctgcccccagcaggaccctctcacatcaactgccagtgctcttccccatgatagggggtgacgtgagaggggtgggactgaaattcagaggacagcgctcgaaggggtttctgggaaacacttgggtggctccgccccctcacacaagggcctcaggttttacccggtaagtccctaagtgcctcaactgccctctcatgtcagctgcctccttgtccctcgatntcgtnaggggacactgtgctattcgatcttgattgtcgaagagtttttaggatggagtaccagcaaaaccaggtggaaataaagttgtctgaacccaaagaaaaaaaaaa

SEQ ID NO: 8 - protein, 883 aa, Mus musculus
MIPLLLSLLAALVLTQAPAALADDLKEDSSEDRAFRVRIGAAQLRGVLGGALAIPCHVHHLRPPRSRRAAPGFPRVKWTFLSGDREVEVLVARGLRVKVNEAYRFRVALPAYPASLTDVSLVLSELRPNDSGVYRCEVQHGIDDSSDAVEVKVKGVVFLYREGSARYAFSFAGAQEACARIGARIATPEQLYAAYLGGYEQCDAGWLSDQTVRYPIQNPREACSGDMDGYPGVRNYGVVGPDDLYDVYCYAEDLNGELFLGAPPSKLTWEEARDYCLERGAQIASTGQLYAAWNGGLDRCSPGWLADGSVRYPIITPSQRCGGGLPGVKTLFLFPNQTGFPSKQNRFNVYCFRDSAHPSASSEASSPASDGLEAIVTVTEKLEELQLPQEAMESESRGAIYSIPISEDGGGGSSTPEDPAEAPRTPLESETQSIAPPTESSEEEGVALEEEERFKDLEALEEEKEQEDLWVWPRELSSPLPTGSETEHSLSQVSPPAQAVLQLDASPSPGPPRFRGPPAETLLPPREWSATSTPGGAREVGGETGSPELSGVPRESEEAGSSSLEDGPSLLPATWAPVGPRELETPSEEKSGRTVLAGTSVQAQPVLPTDSASHGGVAVAPSSGDCIPSPCHNGGTCLEEKEGFRCLCLPGYGGDLCDVGLHFCSPGWEAFQGACYKHFSTRRSWEEAESQCRALGAHLTSICTPEEQDFVNDRYREYQWIGLNDRTIEGDFLWSDGAPLLYENWNPGQPDSYFLSGENCVVMVWHDQGQWSDVPCNYHLSYTCKMGLVSCGPPPQLPLAQIFGRPRLRYAVDTVLRYRCRDGLAQRNLPLIRCQENGLWEAPQISCVPRRPGRALRSMDAPEGPRGQLSRHRKAPLTPPSSL

SEQ ID NO: 9 - DNA, 3977 bp, Mus musculus
  CDS (232)..(2784)
gaattcgcggccgcgtcgaccggatggcgcagttgaactgcgctgctgagctcgcggccgcctgcgcgcgctggggggactcgtctgggctactcccccacctcgcgttggacgaccggtcctggacacaccgcttgcgaggcaagttgaacattgcagagaaggatcttaaggctacacccgacttgccacacttgccatccagctgaagaaccaaaggctgttggagagatggcagtgacatcccaccacatggtccctgtgtttgtcctgatgagcgcctgcctggccaccgcaggtccagagcccagcacccggtgtgaactgtcaccgatcagtgcctctcatccagtccaggccctgatggagagcttcaccgttctgtctggctgtgccagcagaggcaccactgggctgccaagggaggttcacatcctaaacctccgcagtacagaccaaggactaggccagccgcagagagaggttaccctgcatctgaaccccattgcctccgtgcacactcaccacaagcctgttgtgttcctgctcaactccccacagcccctggtgtggcatgtgaagacagagagactggctgctggtgtccccagactcttcctggtttcggagggttctgtggtccagttttcatcaggaaacttctccttgacagcagaaacagaggaaaggagtttccctcaagaaaatgagcatctgctacactgggcccaaaaggaatatggagcagtgacttcattcaccgaacttaagatagcaagaaatatctatattaaagtgggagaagatcaagtgttccctcccacgtgtaacatagggaaaaattttctctcgctcaattaccttgcggagtaccttcaacccaaagccgccgaaggttgtgtcctggccagtcagccccacgagaaggaagtgcatatcattgagttaatctcccccaactccaatccttacagcaccttccaggtggatataataattgacatacgacctgctcgagaggatcctgaggtggtcaaaaacctcgtcctgatcttgaagtgcaaaaaatctgtcaactgggtgatcaagtcttttgacgtcaagggaaacttgaaagttattgctcctgacagtattggctttggaaaagagagtgaacgatccatgacagtgaccaaattggtaagaaatgactacccttccacccaagagaatctgatgaagtgggcattggacaatggctacagcccagtgacgtcatacaccatagctcctgtggccaatagatttcatcttcggcttgagaacaacgaggagatgagagatgaggaagtccacaccattcctcctgagcttcggatcctgctgggccctgaccacctgcctgccctggacagcccacccttccaaggggaaatcccaaatggaggtttcccctttccattcccggatatccccaggagaggctggaaggagggagaagataggatcccccggccaaaggaacccatcattcccagagttcaattgcttccagaccacagagagccagaagaagtgcaagggggcgtgaatatcgccctgtcagtcaaatgtgacaatgaaaagatggtggtagctgtagacaaagattctttccagaccaatggctactcggggatggagctcaccctgttggatccttcctgcaaagccaagatgaatggtacccactttgttctggagtctccgctgaatggctgtggtactagacatcggaggtcagccccagatggtgtggtttactataactctattgtggtgcaggctccatcccctggggatagcagtggctggccagacggctacgaagatttggagtcgggtgataatggatttcctggagacacagatgaaggagaaactgcccccctgagccgtgctggagtggtagtgtttaactgcagcttgcggcagctgaggagtcccagtggcttccaggaccagctcgatggaaatgctaccttcaatatggagctgtataacacagacctctttctggtgccctccccgggggtcttctctgtggcagagaatgagcatgtatatgttgaggtgtctgtcactaaggctgaccaagatctgggatttgccatccaaacctgctttatctctccatactcaaacccagacagaatgtctgattacaccatcatcgagaacatctgtccgaaggatgactctgtgaagttctacagctccaagagagtgcacttccccatcccacatgctgaagtggacaagaagcggttcagctttgtgttcaagtccgtgttcaacacctccctgctcttcctgcactgcgagctgacgctgtgctctaggaacaagggctcccagaagctgccaaagtgtgtgactcctgatgacgcctgcacctctctagatgccaccatgatctggaccatgatgcagaataagaagacattcaccaagcccctggccgtggtcctccaagtagattataaagaaaatgttccaaacatgaaggagtccagtccggttcctcctcctccacagattttccacggcctggacacgctcaccgtgatgggcattgcgtttgcagcatttgtgatcggagcactcctgacgggggccttgtggtatatctactcccacacaggggagacggcacgaaggcagcaagtccctacctcgccaccagcctcggagaacagcagcgcagcccacagcatagggagcactcagagcactccctgctctagcagcagcacggcctaggtggacagatggacgccgctgcctaggtggacagacggatgcctgccaccgccggggcagggccagatgccgatgctgggtgtccagactcagaaggcttgatctgggttccctgtaaagagagagtgaatttcagtatacagacagcctgctctacccaccccttccttaccacggcccacgtaaatgtgaccctggacatctgtcacacgaaagctaagctggtggccttccctgccagcccctcgcaggatgggggtttcaatgtgaaacatatgccagtttttgttctttttttttttttattttttatgctgctttgtccaggtgtccaaacatccatcatttggggtcgtatgttttaaagagtaaaggagacggtgaagggatgtcagctagagcgtagagccagggagagacagccagggattctcgcctagctgaaccaagatgtaaaataggagaaatgctccctccatagccaatctgcatctcgctctggatgcccactcctgacctgccacgccctacatggtcccctgtgtagcttgctagccttgggggcctgtcacttgccaaagtaacattcctctcttgggccaaaatgaatgctgactcctgatactccgcacaagcacaccaactccaaaagtgccttaaagccaggtttgtgtctcctttcaggagagagcgtgtaacgtcagatttgtgcctgtctcgaacattctgtcctaatcatacttaaacatcaggcaggtggggttcagtggtaagcgaagggattattagcaaggtaatgcctgtgtatggtcatataggcacaaatgcagaacacccagcacattggagagacgaccacttcctcctttaaaatgtcattgcttccagagtcggaacctgagttatatgtggtagattatattgactccaaccatcagcctttctgatgagcctttagagaagcctagagagcccgggcatctgatgatgtcacagtgctgcctcagatgatacagagacagggagtttcaggtgaacttcttcagcagaataaggtgatgccactctgacctttgctgagctcagtgactcttcagtgttaaaactgaacccagaggggattctccatgccagatccgctgcaaattaaataataccaattttctagagtccgtcgacgcggccgcgaattc

SEQ ID NO: 10 - protein, 850 aa, Mus musculus
MAVTSHHMVPVFVLMSACLATAGPEPSTRCELSPISASHPVQALMESFTVLSGCASRGTTGLPREVHILNLRSTDQGLGQPQREVTLHLNPIASVHTHHKPVVFLLNSPQPLVWHVKTERLAAGVPRLFLVSEGSVVQFSSGNFSLTAETEERSFPQENEHLLHWAQKEYGAVTSFTELKIARNIYIKVGEDQVFPPTCNIGKNFLSLNYLAEYLQPKAAEGCVLASQPHEKEVHIIELISPNSNPYSTFQVDIIIDIRPAREDPEVVKNLVLILKCKKSVNWVIKSFDVKGNLKVIAPDSIGFGKESERSMTVTKLVRNDYPSTQENLMKWALDNGYSPVTSYTIAPVANRFHLRLENNEEMRDEEVHTIPPELRILLGPDHLPALDSPPFQGEIPNGGFPFPFPDIPRRGWKEGEDRIPRPKEPIIPRVQLLPDHREPEEVQGGVNIALSVKCDNEKMVVAVDKDSFQTNGYSGMELTLLDPSCKAKMNGTHFVLESPLNGCGTRHRRSAPDGVVYYNSIVVQAPSPGDSSGWPDGYEDLESGDNGFPGDTDEGETAPLSRAGVVVFNCSLRQLRSPSGFQDQLDGNATFNMELYNTDLFLVPSPGVFSVAENEHVYVEVSVTKADQDLGFAIQTCFISPYSNPDRMSDYTIIENICPKDDSVKFYSSKRVHFPIPHAEVDKKRFSFVFKSVFNTSLLFLHCELTLCSRNKGSQKLPKCVTPDDACTSLDATMIWTMMQNKKTFTKPLAVVLQVDYKENVPNMKESSPVPPPPQIFHGLDTLTVMGIAFAAFVIGALLTGALWYIYSHTGETARRQQVPTSPPASENSSAAHSIGSTQSTPCSSSSTA

SEQ ID NO: 11 - DNA, 1767 bp, Mus musculus
  CDS (207)..(1271)
gactaatgttggggtaaacacagaaagcccattataacatgataagttcattaaaagaggtcagaatatcgtgcttcagatcacagaagcggtaacgagcagcaccctgctcagaggagaagtgaggggagacaggggcaggagacccagaatctcacgtaacctttaaacttcttcagaaggtcgtgaaaatacatgagataatcatgaaggcaactctcatcttcttccttctggcacaagtctcttgggctggaccatttgaacagagaggcttatttgacttcatgctagaagatgaggcttctggcataatcccttatgaccctgacaatcccctgatatctatgtgcccctaccgatgccagtgtcatcttcgagtggtgcagtgttctgatctgggtttggacaaagtgccctgggattttccacccgacacaaccttgctagacctgcaaaacaacaaaattacagagatcaaagaaggggccttcaagaacctgaaggacttgcataccttgatccttgtcaacaacaagatcagcaaaatcagtccagaggcattcaaacctctcgtgaagttggaaaggctttacctgtctaagaaccaactaaaggaactgcctgaaaaaatgcccagaactctccaggaacttcgtgtccatgagaatgagatcaccaagctgcggaaatccgacttcaatggactgaacaatgtgcttgtcatagaactgggcggcaacccactgaaaaactctgggattgaaaacggagccttccagggactgaagagtctctcatacattcgcatctcagacaccaacataactgcgatccctcaaggtctgcctacttctctcactgaagtgcatctagatggcaacaagatcaccaaggttgatgcacccagcctgaaaggactgattaatttgtctaaactgggattgagcttcaacagcatcaccgttatggagaatggcagtctggccaatgttcctcatctgagggaactccacttggacaacaacaaactcctcagggtgcctgctgggctggcacagcataagtatatccaggtcgtctaccttcacaacaacaacatctccgcagttgggcaaaatgacttctgccgagctggacacccctctcgaaaggcttcctactcggctgtgagtctttacggcaaccctgtccggtattgggaaatctttccaaacaccttcagatgtgtctatgtgcgttctgccattcaacttggaaactacaagtaaccctcagacggcctaattcttataatctggaaaaacaccccaatatgtcaatatcattgctaaaaagaaaaaatactaaaaaagaaagaaagaatgctagattctgaaaattcaagcacactgtgcatgccttttccacatgacttattatgcaagctgaatatacagattgaataattgcctacaataaaaatattttacttgtagtattcagaatcacttttaaagttgtctgtagttgtgaactgagttatcaaagtctgatgtaatcataaatgtcaaccacttagtaaagcgttaaaaggaacagaatacaaagtctctgtaattcatagagctaaattaactcttttgacataaagtcaaatgccgccgaactctagcaatgtattaatctcctttattattggtgaagccttatttgagtatgtaagttcaatatgggctatgtaaaattttcactgtcatattgaaaaaaataaaattttaaaaatgc

SEQ ID NO: 12 - protein, 354 aa, Mus musculus
MKATLIFFLLAQVSWAGPFEQRGLFDFMLEDEASGIIPYDPDNPLISMCPYRCQCHLRVVQCSDLGLDKVPWDFPPDTTLLDLQNNKITEIKEGAFKNLKDLHTLILVNNKISKISPEAFKPLVKLERLYLSKNQLKELPEKMPRTLQELRVHENEITKLRKSDFNGLNNVLVIELGGNPLKNSGIENGAFQGLKSLSYIRISDTNITAIPQGLPTSLTEVHLDGNKITKVDAPSLKGLINLSKLGLSFNSITVMENGSLANVPHLRELHLDNNKLLRVPAGLAQHKYIQVVYLHNNNISAVGQNDFCRAGHPSRKASYSAVSLYGNPVRYWEIFPNTFRCVYVRSAIQLGNYK

SEQ ID NO: 13 - DNA, 1474 bp, Mus musculus
  CDS (152)..(556)
gcctctgtctccctctctccacgaactgcccaggagcgagcagctgctcccggttggccctgacggacagacaaaccgacagcctgacaacctagtccaccaactaagcagcctgcacctggctgcttgtccctccccaggaacattgaccatgtgtcccctgtggctactcaccttgctgctggccctgagccaggccttgccctttgagcagaagggtttctgggacttcaccttggatgatgggctgctcatgatgaatgatgaggaggcttcaggttcagacaccacttcaggtgtccccgacctggactctgtcacacctaccttcagtgccatgtgtcctttcggttgccactgccacctgcgggttgttcagtgctctgacttgggtctgaagactgtgcccaaggagatctcacctgacaccacactgctagacctgcagaacaatgacatttctgagcttcgcaaggatgacttcaaaggcctccagcacctctacgtaagcctcctcctgcctcctgccttttctgaggagccaggaggagtttgatgttcctatgacttggaagaagggtactcacaaatgaggttgggaagaggggtgggaaccgatggagtcctgagataagtctggagctggctgtaatgagaacgcccaaaaccatgcattatatgtatgggtacaagacgaaatctgtcaggtcccatttttttttgacagaaatctttacgttttgttgactcatctgaacaacacagaaagtgtattccatcatagctcttagagaaactgtctagcaaagtatgctaggcagtgatggcattaccagggcagcttggagctaacctcagctccttgccctacactctactcaccaacacatactgatctccatgagaccattaaaggttcagatgtttcccaagtgccatcagtagcaagttcaggaggcagaggagaggggaggtggagtggggggctggggtgctcaccctggttgcctgccccgttttaggccctggtcttggtaaacaataagatctccaagatccatgagaaggcctttagccctctgcggaagctgcaaaaactctacatctccaagaaccacctggtggagattcctcccaacctgcccagctccctggtagaactacgaatccatgacaaccgtatccgcaaagtgcccaagggcgtgttcagcgggctccggaacatgaactgcattggtgagactagtctgtaccttagcatcaggcataaaggacacaggtagaggatatctctttcagccagggcagctgggtccatgtagtcatggaaaagccctgtgtgcctcagaatgttccagatgtttgttttgggacatgaagaaaaagactgtggtggctataaaggctcggttgaagaaagagtctcaaatatagtctaaaaaagaccagtatgtttctgaaaaaaaaaaaaaaa

SEQ ID NO: 14 - protein, 134 aa, Mus musculus
MCPLWLLTLLLALSQALPFEQKGFWDFTLDDGLLMMNDEEASGSDTTSGVPDLDSVTPTFSAMCPFGCHCHLRVVQCSDLGLKTVPKEISPDTTLLDLQNNDISELRKDDFKGLQHLYVSLLLPPAFSEEPGGV

SEQ ID NO: 15 - DNA, 2860 bp, Mus musculus
  CDS (78)..(1208)
gctgccacattctccaacccaaggagaccagacagaaggacgtggtcactctgaacagttcaacccaagagacaaaaatgcagtgggcctccgtcctgctgctggctgggctctgctcactttcccagggccagtatgatgaagactctcactggtggatccaatacctccgaaaccagcagtccacctactacgacccctatgacccttacccctatgagccctctgagccttacccctatggagtggaagaaggcccagcctatgcctatggtgcaccacctccaccagagccccgtgattgtccccaagaatgcgactgtccccccaacttccccacagccatgtactgtgacaaccgcaacctcaagtacctgccttttgtgccctcccgcatgaagtacgtctacttccagaacaaccagatctctgccatccaggaaggtgtctttgacaatgccactgggctcctctgggtcgctctacatggcaaccagattaccagtgacaaggtaggcaggaaagtcttctccaagctgaggcacttggagaggctgtacttggaccacaacaacctgacccggatgcccggcccgctgccccgatccctgagagagctccacctggaccacaaccagatctcacgggtccccaacaatgctttggagggcctggagaacctcacggccttatatctccaccacaatgagatccaggaagtggggagttccatgagaggcctccggtccctgatcctactagacctgagttataaccaccttcggagggtacccgacggtctgccctcagccctggagcagctgtacctagaacacaacaatgtctacaccgtccctgacagctacttccgggggtcacccaagctgctgtacgtccggttgtctcacaacagtctcactaacaacggccttgctaccaacaccttcaactccagcagccttcttgagctagacttgtcttacaatcagctgcagaagatccctcctgtcaacaccaacctggagaatctttacctccagggcaacaggatcaatgagttctccatcagcagcttctgcacggtggtggacgtcatgaacttctccaagctgcaggtgctacgcctggatgggaacgagatcaagcgcagcgcaatgccggtggacgccccactctgcctgcgcctcgccaacctcatcgagatctgaggggcctcgccttgggctcctggctccgggctccgagtaccgtgcaccgtgcaccgggctcggggtacatgccctgccaccccgctgcatgtttggcttttgctggatggtctgggacacgcatgtgacagaagtccacaggatcttattcaatctccttccaacaggcagagttaggtgggatcaggggccaggccagtttctgcagggggatgaatttggtggtaaaagaaatatagctatagagtctagccccaaaatcttcttgcttggacagtagcatataaccagttccaatttgaccttttttgagctgtgctcaacagcatggccagctgcttctgcagttgctctgggctcctgctctttgctcctcaaaacatcacaacacacctcttgcccagtcacctcctcccagccccagctcaccctctctgccctctttcactggggcctcttctaatgtcttaggcagtcaggagacacccacaccctaagggcacactcttatctgaggctaaacggatggctaaaatcacacacttacccccacctcacctgtttaaagtcaccatattgaacaccataatgatgagctgttaacataggggataaccgactctataatggaggatctatcagaggagggaagtgtccagtggtcattatcagtatccatagtaagatggggaagagacacacctcaggatagcagaactgaaggggcagccccctttccagtttcatctgacacaacatgatctgcaccccttcttgggcttttagtatcgaaggggcaagcgtggttttcaaaacatgagaaagagcctctgctcatcctttggtctagtatgaagggttgttacgcaaatggcagaggcagactccacccgccgaggacctacccactaacccagactagggggtgatctcaatagctcgtttgtccataatgctagagcatcaggaaccagccctgtggagatgctcactggggtgcaagaagccctgccaggcccaagtcctgccgacactagacctggcttcctctgcattcacactatgcagtccgccttatgcccagtggtgcctgtgatttgtagtttgtctaagaacagccccatcttctcctttttaaaggtgatattgcctaggtacaaccaacagcttcttctagaccccactgggatagagccaccagggtagcgagtggcaagaagcagttgtaagatgctagcctgaggtgggtccctgccttagacccctgactctgtgtgcacttgggagcaagcttctctggaaaggacagggtggggcggggtgcttagactgtgctacgggttccagaactgcaatccacaaaagccaaaccagcttgtttcaaccggggagtgccacctgcggagcaaggctgccctggccagggttcttgggaagcacctgcatgtatcaccatcctgcctctgaagagccctcagcatgaagcaagcatgaatggcggccaacctaaccaataaagttattttataagtgcatctgcaaggatggaatggttggtggcagccctcggcatcgcctcggggtacctagacacacaggccttgaaccgtgtcagctctttataaagagtagcctatataaaaactcaagtt

SEQ ID NO: 16 - protein, 376 aa, Mus musculus
MQWASVLLLAGLCSLSQGQYDEDSHWWIQYLRNQQSTYYDPYDPYPYEPSEPYPYGVEEGPAYAYGAPPPPEPRDCPQECDCPPNFPTAMYCDNRNLKYLPFVPSRMKYVYFQNNQISAIQEGVFDNATGLLWVALHGNQITSDKVGRKVFSKLRHLERLYLDHNNLTRMPGPLPRSLRELHLDHNQISRVPNNALEGLENLTALYLHHNEIQEVGSSMRGLRSLILLDLSYNHLRRVPDGLPSALEQLYLEHNNVYTVPDSYFRGSPKLLYVRLSHNSLTNNGLATNTFNSSSLLELDLSYNQLQKIPPVNTNLENLYLQGNRINEFSISSFCTVVDVMNFSKLQVLRLDGNEIKRSAMPVDAPLCLRLANLIEI

SEQ ID NO: 17 - DNA, 1723 bp, Mus musculus
  CDS (125)..(1093)
gacagttggaagatgacagttgaccagaattcctatccattggtcaggggcaaataccaaggactctgtatgctcagtcactctgtgcgacttcatcaagtcggagccaaagaaatcttgaaagatgggaatgctagcaagagttgccctgggacttatcatcattgatgctgtattggctgcaccaactacagagctctttaactatgactcagaagtgtatgatgccatcttggaggacacagggaccttttataactatgaacacattcctgacaaccatgtagagaacgagaaagtgtctgagaggctttctgggaacagagagctcctgaccccaggcccacagctaggggacaaccaggatgaagacaaggatgaagaatctactcccaggctgatcgatggctcttccccacaggagccagaattcccggggcttctgggtccacacaccaacgaagactttccaacttgtctcctgtgtacttgtataagtaccactgtatactgtgatgaccatgaactggacgctattcctccactgcccaagaagactacatatttctattcacgttttaacagaattaaaaagatcaacaaaaatgactttgcaagcttaaatgatttaaaaaggatcgatctgacatcaaatttaatatcggagattgatgaagatgcattccgaaagctgcctcacctccaagagctggttctgcgtgacaataagataaagcagcttccagaattgccaaacactttgacatttattgatatcagcaacaatagactgggaagaaaaggcattaaacaagaagcttttaaagatatgtatgatctccaccatctctatatcaccgataacagcttggaccacatccctctaccactcccagaaagtctgcgggctcttcacctccagaataatgacattctggagatgcatgaagatactttctgcaatgttaaaaatttaacttatgttcgtaaggcattagaggatattcgactggatggaaaccctatcaacctcagcagaactcctcaagcctacatgtgtctacctcgtttgcccattgggagttttatctaatgttaggatgctaggcaaatatcagaattgtgatgctttctgtagccaagagaagcatatatctcttcagaagcaaactcagtatatttgagatgcgtttaaaacatgtggcacaatgatataaacaataagctaaaggtgttgagataaaataagagttcaataatttaattaaacatgattaggtataaattaacaaaatatatagacagtaagttaaataaaatgtatgccaactagtcatcaaatctcatcaagaattgtaggtgttcatgtcaagttaaatatgtaagaaataaaatattggtaatgaatactttagatacgttgctaagatgtagacattttaattaaacttctctagtctttattttcactcatgtatgtttcccacctgattcagttaagagcagaaaaaaatattaaatgtgtttgaaagatatatggtcttgtgaaaaagtatttcttataattgtagtgtgcaaataactatgtaatagcagtatacattccttcagtttctaatgacatctatcatctataagaacatcaactcattttgcaaacttggttagttgacagctacctgtattcctctacatacaaaaaataaaatatgagatatagagc

SEQ ID NO: 18 - protein, 322 aa, Mus musculus
MGMLARVALGLIIIDAVLAAPTTELFNYDSEVYDAILEDTGTFYNYEHIPDNHVENEKVSERLSGNRELLTPGPQLGDNQDEDKDEESTPRLIDGSSPQEPEFPGLLGPHTNEDFPTCLLCTCISTTVYCDDHELDAIPPLPKKTTYFYSRFNRIKKINKNDFASLNDLKRIDLTSNLISEIDEDAFRKLPHLQELVLRDNKIKQLPELPNTLTFIDISNNRLGRKGIKQEAFKDMYDLHHLYITDNSLDHIPLPLPESLRALHLQNNDILEMHEDTFCNVKNLTYVRKALEDIRLDGNPINLSRTPQAYMCLPRLPIGSFI

SEQ ID NO: 19 - DNA, 4909 bp, Mus musculus
  CDS (390)..(3296)
gcagctccgagctaggtgctatcgcaaggccagagcgcacagcccggcggagagagcagatccttgctcagatcgagtcaaatcggggccaaggcggaggacgaagagtccaggctcctattctggacttgttccccagctccgggggcgcttctaggtcctgcagcagccaggagtgcggagccaccaactcggtgctggaatgaaaaaattcccgcgcgccagtgcagaatctttctaagtgacccggagcttcgggtgctagctctgcacgaactttcccatcaaagtgatcgtgaattttaagcatcaggagcaggccagcgaagctctacgcgtctaaacgtctatccagaccaagagttctctgcggtgcagggtgcggtgccatgcagccaaaagtccctttggggtcacgcaagcagaagccctgctccgacatgggggacgtccagcgggcagcgagatctcggggctctctgtccgcacacatgctgttgctgctcctcgcttccataacaatgctgctatgtgcgcggggcgcacacgggcgccccacggaggaagatgaggagctggtcctgccctcgctggagcgcgccccgggccacgattccaccaccacacgccttcgtctggacgcctttggccagcagctacatctgaagttgcagccggacagcggtttcttggcgcctggcttcaccctgcagactgtggggcgcagtcccgggtccgaggcacaacatctggaccccaccggggacctggctcactgcttctactctggcacggtgaacggtgatcccggctctgccgcagccctcagcctctgtgaaggtgtgcgtggtgccttctacctacaaggagaggagttcttcattcagccagcgcctggagtggccaccgagcgcctggcccctgccgtgcccgaggaggagtcatccgcacggccgcagttccacatcctgaggcgaaggcggcggggcagtggcggcgccaagtgcggcgtcatggacgacgagaccctgccaaccagcgactcgcgacccgagagccagaacacccggaaccagtggcctgtgcgggaccccacgcctcaggacgcgggaaagccatcaggaccaggaagcataaggaagaagcgatttgtgtccagcccccgttatgtggaaaccatgctcgtggctgaccagtccatggccgacttccacggcagcggtctaaagcattaccttctaaccctgttctcggtggcagccaggttttacaagcatcccagcattaggaattcaattagcctggtggtggtgaagatcttggtcatatatgaggagcagaagggaccagaagttacctccaatgcagctctcacccttcggaatttctgcaactggcagaaacaacacaacagccccagtgaccgggatccagagcactatgacactgcaattctgttcaccagacaggatttatgtggctcccacacgtgtgacactctcgggatggcagatgttggaactgtatgtgaccccagcaggagctgctcagtcatagaagatgatggtttgcaagccgccttcaccacagcccacgaattgggccatgtgtttaacatgccgcacgatgatgctaagcactgtgccagcttgaatggtgtgactggcgattctcatctgatggcctcgatgctctccagcttagaccatagccagccctggtcaccttgcagtgcctacatggtcacgtccttcctagataatggacacggggaatgtttgatggacaagccccagaatccaatcaagctcccttctgatcttcccggtaccttgtacgatgccaaccgccagtgtcagtttacattcggagaggaatccaagcactgccctgatgcagccagcacatgtactaccctgtggtgcactggcacctccggtggcttactggtgtgccaaacaaaacacttcccttgggcagatggcaccagctgtggagaagggaagtggtgtgtcagtggcaagtgcgtgaacaagacagacatgaagcattttgctactcctgttcatggaagctggggaccatggggaccgtggggagactgctcaagaacctgtggtggtggagttcaatacacaatgagagaatgtgacaacccagtcccaaagaacggagggaagtactgtgaaggcaaacgagtccgctacaggtcctgtaacatcgaggactgtccagacaataacggaaaaacgttcagagaggagcagtgcgaggcgcacaatgagttttccaaagcttcctttgggaatgagcccactgtagagtggacacccaagtacgccggcgtctcgccaaaggacaggtgcaagctcacctgtgaagccaaaggcattggctactttttcgtcttacagcccaaggttgtagatggcactccctgtagtccagactctacctctgtctgtgtgcaagggcagtgtgtgaaagctggctgtgatcgcatcatagactccaaaaagaagtttgataagtgtggcgtttgtggaggaaacggttccacatgcaagaagatgtcaggaatagtcactagtacaagacctgggtatcatgacattgtcacaattcctgctggagccaccaacattgaagtgaaacatcggaatcaaagggggtccagaaacaatggcagctttctggctattagagccgctgatggtacctatattctgaatggaaacttcactctgtccacactagagcaagacctcacctacaaaggtactgtcttaaggtacagtggttcctcggctgcgctggagagaatccgcagctttagtccactcaaagaacccttaaccatccaggttcttatggtaggccatgctctccgacccaaaattaaattcacctactttatgaagaagaagacagagtcattcaacgccattcccacattttctgagtgggtgattgaagagtggggggagtgctccaagacatgcggctcaggttggcagagaagagtagtgcagtgcagagacattaatggacaccctgcttccgaatgtgcaaaggaagtgaagccagccagtaccagaccttgtgcagaccttccttgcccacactggcaggtgggggattggtcaccatgttccaaaacttgcgggaagggttacaagaagagaaccttgaaatgtgtgtcccacgatgggggcgtgttatcaaatgagagctgtgatcctttgaagaagccaaagcattacattgacttttgcacactgacacagtgcagttaagaggcgttagaggacaaggtagcgtggggagggctgatacactgagtgctggagggatccagtgagtcaaaccagtaagcagtgaggtgtggcaaggaggtgtgtgtaggggatacatagcaaaggaggtagatcaggacactaccctgccagttacattctgataaggtagttaatgaggcacagtagcatctgaaagaccatacagagcactaaggagccccaaagcactattagtatctcttttcttatatctatcgcccaaataattttcagagtctggcagaagccctgttgcactgtactgactagatacttcttatcacaaagattgggaaaggcaaagcagaaagatggtaagactgggtttcaaacaaggcttggtttctatcactggaggcaaggaggaggggacaaacaagatcattattcgaagtcgctggttgctgtggttttacggaaggttgatgcatcattcctatcaacagtgaaaagttcagcttgttcaacgtgacagaaaggctcatctccgtgaaagagctcctgatttcttcttacaccatctcagttcttaactataattcatgttgaggtagaaacaattcatctatttataaaatgtacattggaaaaaaaaaagtgaagtttatgaggtacacataaaaactgaaggaaacaatgagcaacatgcctcctgctttgcttcctcctgaggtaaacctgcctggcgattgaggttgtttaagattatccatggctcacaagaggcagtaaaataatacatgttgtgccagagttagaatggggtatagagatcagggtcccatgagatggggaacatggtgatcactcatctcacatgggaggctgctgcagggtagcaggtccactcctggcagctggtccaacagtcgtatcctggtgaatgtctgttcagctcttctactgagagagaatatgactgtttccatatgtatatgtatatagtaaaatatgttactatgaattgcatgtactttataagtattggtgtgtctgttccttctaagaaggactatagtttataataaatgcctataataacatatttatttttatacatttatttctaatgataaaacctttaagttatatcgcttttgtaaaagtgcatataaaaatagagtatttatacaatatatgttaactagaaataataaaagaacacttttgaatgtgtatgcctattttctggagtgggattaacttctgggcaagaaatctgatgagacacaaacattggacttcaagacagttttaaattttgggtaaatgaactgtatttcctgtttatagacgtactaataaaaaagaagttgatgatgtctttagtggtaagattgttactaatgtggttggcaaattgctgtaaagagccagatagtaagcatttatggcattgtaggctatctttcctgccacaaccatgtgacagtgagtgctttgtaggactgagagcagccataaatgacatgtaaatgataaactgtggctgtgctttaataaaactttatttacaaaaacaaaaaaaaaaaaaaaaaaaaaaaaaaaaaaaaaaaaaaaaaaaaaaaaaa

SEQ ID NO: 20 - protein, 968 aa, Mus musculus
MQPKVPLGSRKQKPCSDMGDVQRAARSRGSLSAHMLLLLLASITMLLCARGAHGRPTEEDEELVLPSLERAPGHDSTTTRLRLDAFGQQLHLKLQPDSGFLAPGFTLQTVGRSPGSEAQHLDPTGDLAHCFYSGTVNGDPGSAAALSLCEGVRGAFYLQGEEFFIQPAPGVATERLAPAVPEEESSARPQFHILRRRRRGSGGAKCGVMDDETLPTSDSRPESQNTRNQWPVRDPTPQDAGKPSGPGSIRKKRFVSSPRYVETMLVADQSMADFHGSGLKHYLLTLFSVAARFYKHPSIRNSISLVVVKILVIYEEQKGPEVTSNAALTLRNFCNWQKQHNSPSDRDPEHYDTAILFTRQDLCGSHTCDTLGMADVGTVCDPSRSCSVIEDDGLQAAFTTAHELGHVFNMPHDDAKHCASLNGVTGDSHLMASMLSSLDHSQPWSPCSAYMVTSFLDNGHGECLMDKPQNPIKLPSDLPGTLYDANRQCQFTFGEESKHCPDAASTCTTLWCTGTSGGLLVCQTKHFPWADGTSCGEGKWCVSGKCVNKTDMKHFATPVHGSWGPWGPWGDCSRTCGGGVQYTMRECDNPVPKNGGKYCEGKRVRYRSCNIEDCPDNNGKTFREEQCEAHNEFSKASFGNEPTVEWTPKYAGVSPKDRCKLTCEAKGIGYFFVLQPKVVDGTPCSPDSTSVCVQGQCVKAGCDRIIDSKKKFDKCGVCGGNGSTCKKMSGIVTSTRPGYHDIVTIPAGATNIEVKHRNQRGSRNNGSFLAIRAADGTYILNGNFTLSTLEQDLTYKGTVLRYSGSSAALERIRSFSPLKEPLTIQVLMVGHALRPKIKFTYFMKKKTESFNAIPTFSEWVIEEWGECSKTCGSGWQRRVVQCRDINGHPASECAKEVKPASTRPCADLPCPHWQVGDWSPCSKTCGKGYKKRTLKCVSHDGGVLSNESCDPLKKPKHYIDFCTLTQCS

SEQ ID NO: 21 - DNA, 3673 bp, Mus musculus
  CDS (392)..(2929)
ggggagaacccggggaagacccacagatacacagaaacgagagagacagaagaggagagagacagagacaaagagacagcagcaggaggaggaggaggaggaggcaaagacagggcgagcactgaggcaaagccagacagctgagcagagggagaagccaaggagtcacagacaaagacctgggacagaaaggcaaaggaggaagttttggagcagaaagaacccgtgggcacttttcctgagcccaggggctgttttctttttcatcttctctgaaagtccctagcccatttaaaaactttgcctcagattttggcacaagccccaaagcccttctggctttaacgaggagccttccacagttagggtgtggagcattttggtgccgcagatggcctcaatccatcccagctgcagcccgggtaccatgtcccagatgggcttgcatcccaggaggggcttgactgggcactggctgcaaagattccaaccctgcttgccgcttcacactgtgcagtggcggaggctgctgctgctggccttcctcctgtccttagcgtggcccgccagccccctcccccgggaggaggagatcgtgtttccagagaagctcaatggcagtagcatcctacctggatcaggcgttcctgccaggctgctgtaccgattgccagcctttggggagatgttgctactagaactagaacaggaccctggggtgcaggtagagggtttgactgtacagtacctgggccaggcacctgagatgctgggtggggcagagccaggtacctacctgactggcaccatcaatggagatccggagtcggtggcatctctgcactgggacgggggagccctattaggggtactgcagtaccgtggggccgaactccacctccagcctctggaaggaggcgcccttaactctgctgggggaccgggggctcacatcctacgccggaagagtcctgccagcagccaaggtcccatgtgcaccgtcaaggctccttctgggagcccgagtcccatttcccgcagaaccaagcgcttcgcttctctgagtagattcgtggagacactggtggtagcagatgacaagatggcagcattccatggtacagggttaaagcgctacctgctgacggttatggcagctgccgctaaagcctttaaacacccaagcatccgaaaccctgtcaacttggtggtgacgcgcctggtgatcctggggtccggccaggaagggccccaagtggggccaagtgccgcccagaccctacgcagcttctgcacctggcagcggggcctcaacacccctaacgactcagatcctgaccactttgacacagccattctgttcacccggcaggacctgtgtggggtctccacttgtgacaccctgggtatggctgatgtgggcacagtgtgtgatccagctaggagctgtgctattgtggaagatgatgggctccagtcagccttcactgctgctcatgaactgggccatgtcttcaacatgctccatgataactccaagccatgcactaacttgaatgggcaggggggttcctctcgccatgtcatggctcctgtcatggcccatgtggaccctgaagagccctggtcgccctgcagtgcccgattcatcactgacttcctggacaatggttatgggcactgcctcttagacaaaccggaggctcccctccatctaccagcgacttttcctggcaaggactatgacgctgaccgccaatgccaactgaccttcggtcctgactcaagccattgtccacagctgccaccgccctgtgctgccctctggtgctctggccacctcaatggccatgccatgtgccagacgaagcactcaccttgggctgatggcactccctgcgggtcttcacaggcctgcatgggtggccgctgtctgcacgtggaccagctcaaggacttcaatgttcctcaggctggaggctggggcccctggggaccatggggtgactgctccaggacttgtgggggtggtgtccagttctcctcccgggattgcacgaggcccgtcccccggaacggtggcaagtattgtgagggccgccggactcgcttccgctcctgcaacacggagaactgcccacacggctcagcattgaccttccgtgaagagcagtgtgctgcctacaaccaccgaaccgacctcttcaagagctttccagggcccatggactgggttccgcgctacacaggtgtggcccctcgagaccaatgcaaactcacctgccaggcccgggcactgggctactactacgtattggagccccgggtggcagatgggactccctgctccccagacacctcctctgtctgtgtccagggccgctgtatccatgctggctgtgaccggatcattggctccaaaaagaaatttgacaagtgcatggtgtgcggcggggatggctctcgctgcagcaagcagtcgggctccttcaaaaaattcaggtatggatacagcgatgtggtcacgatccctgcgggggccacccatatccttgtacggcagcagggggggtctggtctcaagagcatctacctggccctgaagctttctgacggttcttacgccctcaatggtgaatacacgctgatgccctccccaacagatgtggttcttcctggggcagtcagcttgcgctacagcggagccacagcagcctcagagacactgtctggacatgggccgctggcccagcccttgacgctgcaagtcctggtggctggcaacccacagaatgcacgtctgcggtacagtttctttgtcccgcggccagtcccttcaacaccacgccctcctccccaagactggctgcaacgcagggcagagatactgaagatccttcggaagcgtccctgggcaggccggaaataacctcactgtcccggctgccctttttgggcgccggggcctcggactcatctgggagaatgagcaggcttctgcaactgcctcctgctaaaacacagtagggaggtgtagagggtgagatctgcctgcctcactgccccaaaccgcaggctggccctgccctggcttcctgccctgggaggcagtgatgtcttggtgaatggaaaggggctaggtgacagtaccctatctactaaactgccccctctaccctgcaggtcacaggaggaatggggggaagacagggtgggtcctgggccctagttgtatttatttggtatttattcatttttatttagcaccaggaaaggggactagggtcttggggaaactcacctattatagccctaacctagctatgaaatccagggtgttggtgacaaatatgagtggtgtgtgtgtgtgtgtgtgtgtgtgtgtgtgtgtttatgtatgaggtacaacctgccctgctttcctctccctaatttttttttttttctgggaaaaggaaagtcaaaggtaggactgccttcagggagtaagggatgattgtgtttttaaattgaagtttgctatttatatgctctttttggagtcagacaaatgtgggttatattctggccccgcatctttgagcattagttttctcatgtgccaataataatcccttagaaattggttgtaaggattaaatgatgttaataaagaactagcatagagcctctc

SEQ ID NO: 22 - protein, 845 aa, Mus musculus
MASIHPSCSPGTMSQMGLHPRRGLTGHWLQRFQPCLPLHTVQWRRLLLLAFLLSLAWPASPLPREEEIVFPEKLNGSSILPGSGVPARLLYRLPAFGEMLLLELEQDPGVQVEGLTVQYLGQAPEMLGGAEPGTYLTGTINGDPESVASLHWDGGALLGVLQYRGAELHLQPLEGGALNSAGGPGAHILRRKSPASSQGPMCTVKAPSGSPSPISRRTKRFASLSRFVETLVVADDKMAAFHGTGLKRYLLTVMAAAAKAFKHPSIRNPVNLVVTRLVILGSGQEGPQVGPSAAQTLRSFCTWQRGLNTPNDSDPDHFDTAILFTRQDLCGVSTCDTLGMADVGTVCDPARSCAIVEDDGLQSAFTAAHELGHVFNMLHDNSKPCTNLNGQGGSSRHVMAPVMAHVDPEEPWSPCSARFITDFLDNGYGHCLLDKPEAPLHLPATFPGKDYDADRQCQLTFGPDSSHCPQLPPPCAALWCSGHLNGHAMCQTKHSPWADGTPCGSSQACMGGRCLHVDQLKDFNVPQAGGWGPWGPWGDCSRTCGGGVQFSSRDCTRPVPRNGGKYCEGRRTRFRSCNTENCPHGSALTFREEQCAAYNHRTDLFKSFPGPMDWVPRYTGVAPRDQCKLTCQARALGYYYVLEPRVADGTPCSPDTSSVCVQGRCIHAGCDRIIGSKKKFDKCMVCGGDGSRCSKQSGSFKKFRYGYSDVVTIPAGATHILVRQQGGSGLKSIYLALKLSDGSYALNGEYTLMPSPTDVVLPGAVSLRYSGATAASETLSGHGPLAQPLTLQVLVAGNPQNARLRYSFFVPRPVPSTPRPPPQDWLQRRAEILKILRKRPWAGRK

SEQ ID NO: 23 - DNA, 3002 bp, Mus musculus
  CDS (18)..(2810)
ccggcgggcagcgcactatgcggctcgagtgggcgtccttgttgctgctactgctgctgctgagcgcgtcctgcctgtccctggccgctgacagccccgccgcggcacctgcccaggataaaaccaggcagcctcaggctgcagcagcggccgccgagccggaccagccgcagggggaggaaacacgggagcgaggccatttacaacccttggccgggcagcgcaggagcggcgggctggtccataatatagaccaactctactctggcggtggcaaagtgggctaccttgtctacgcgggcggccggaggttcctgctggacctggagagagatgacacagtgggtgctgctggtagcatcgttactgcaggaggagggctgagcgcatcctctggccaccggggtcactgtttctacagaggcaccgtggacggcagccctcgatccctagctgtctttgacctctgcgggggtctcgatggcttctttgcagtcaagcatgcgcgctacactctaaagccactcctgcgtgggtcctgggcagagtatgaacgaatttatggggatggatcttcccgcatcctgcatgtctacaaccgcgagggctttagcttcgaggccctgccgccacgcgccagttgcgagactcctgcatccccatctgggccccaagagagcccctcggtgcacagtagatctaggagacgctcagcgctggccccgcagctgctggaccactcagctttctcgccatctgggaacgcgggacctcagacttggtggaggcgtaggcgccgttccatctccagggcccgccaggtggagctcctcttggtggctgactcgtccatggccaggatgtatgggcggggcctgcagcattacctgctgaccctggcctccatcgccaacaggctgtacagtcatgcaagcattgagaaccacatccgcctggcggtggtgaaggtggtggtgctgacggacaaggacacgagtctggaggtgagcaagaatgcggccacgaccctcaagaacttttgcaaatggcagcaccaacataaccagctaggggatgatcacgaagagcactacgatgcagccatcctgttcacccgagaggatttatgtgggcatcattcatgtgacaccctgggaatggcagacgttgggaccatatgttctccggagcgcagctgtgcagtgattgaagatgatggcctccatgcagccttcactgtggctcatgaaattgggcatctacttggcctttctcatgacgattccaaattctgtgaagagaacttcggtactacagaagacaagcgtttaatgtcttcaatccttaccagcatcgatgcatccaagccctggtccaaatgcacgtcagccaccatcacagaattcctggatgatggtcatggtaattgtttgctagacctaccacggaagcagattttgggtcccgaggaactcccaggacagacctacgatgccacccagcagtgcaacttgacatttgggcctgagtactcggtgtgccctggcatggatgtctgtgcgcggctgtggtgtgctgtggtgcgccaaggccaaatggtgtgtctgaccaagaagctgccggctgtggagggcactccctgtgggaagggaagagtctgccttcaaggcaaatgtgtggacaaaactaagaaaaaatattactcgacatcaagccatggaaattgggggtcctggggcccctggggtcagtgttctcgctcatgcgggggaggagtgcagtttgcctaccgccattgtaataaccctgcacctcgaaacagtggccgctactgcacagggaagagggccatataccgttcctgcagtgttacaccctgcccacccaatggtaaatcttttcgccatgagcagtgtgaagccaaaaatggctatcagtctgatgcaaaaggagtcaaaacatttgtagaatgggttcccaaatatgcaggtgtcctgccggcagatgtgtgcaagcttacctgcagagctaagggcacaggctactatgtggtcttttctccaaaggttacggatgggactgaatgcaggccgtacagcaactctgtgtgtgtccgaggacggtgtgtgagaactggatgtgacggcattattggctcaaagctacaatatgacaagtgtggagtgtgcggaggggataactccagttgtacaaagattatcggaaccttcaataaaaaaagcaagggttatactgacgttgtgaggatccctgaaggagcaacccacataaaagtccgacagttcaaagccaaagaccagactagattccctgcctacttagccctgaagaagaaaactggcgagtaccttatcaatggcaagtacatgatttccacttcagagaccatcatcgacatcaatggtaccgtcatgaactacagtggatggagccacagagatgattttttacatgggatgggctattcagccacaaaagaaatcctgatcgtgcagatccttgccacagacccaactaaagcgctaggcgtccgttacagcttttttgttcccaagaagaccactcaaaaagtaaactctgtcatcagccatggcagcaacaaggtgggaccacactctacacagctgcagtgggtgacaggtccatggctggcctgctccaggacctgtgacacaggctggcacactaggaccgtgcagtgccaggatggaaacaggaaattagctaaaggatgccttctctctcagaggccttctgcatttaagcaatgtctgctgaagaaatgttagcctgtggtttactctaatgcacaaaaaaacaacaggaggatcatcgcagatacagctgtggtgaagacaaggcctacccaaagcacagaaagtcatgccttcatgtcattgtcaccacgagtcgaattatgggcagaatctgctctctgcgaccaaaaggtttactctacttggtgaatgatggtaccgtga

SEQ ID NO: 24 - protein, 930 aa, Mus musculus
MRLEWASLLLLLLLLSASCLSLAADSPAAAPAQDKTRQPQAAAAAAEPDQPQGEETRERGHLQPLAGQRRSGGLVHNIDQLYSGGGKVGYLVYAGGRRFLLDLERDDTVGAAGSIVTAGGGLSASSGHRGHCFYRGTVDGSPRSLAVFDLCGGLDGFFAVKHARYTLKPLLRGSWAEYERIYGDGSSRILHVYNREGFSFEALPPRASCETPASPSGPQESPSVHSRSRRRSALAPQLLDHSAFSPSGNAGPQTWWRRRRRSISRARQVELLLVADSSMARMYGRGLQHYLLTLASIANRLYSHASIENHIRLAVVKVVVLTDKDTSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEENFGTTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRVCLQGKCVDKTKKKYYSTSSHGNWGSWGPWGQCSRSCGGGVQFAYRHCNNPAPRNSGRYCTGKRAIYRSCSVTPCPPNGKSFRHEQCEAKNGYQSDAKGVKTFVEWVPKYAGVLPADVCKLTCRAKGTGYYVVFSPKVTDGTECRPYSNSVCVRGRCVRTGCDGIIGSKLQYDKCGVCGGDNSSCTKIIGTFNKKSKGYTDVVRIPEGATHIKVRQFKAKDQTRFPAYLALKKKTGEYLINGKYMISTSETIIDINGTVMNYSGWSHRDDFLHGMGYSATKEILIVQILATDPTKALGVRYSFFVPKKTTQKVNSVISHGSNKVGPHSTQLQWVTGPWLACSRTCDTGWHTRTVQCQDGNRKLAKGCLLSQRPSAFKQCLLKKC

SEQ ID NO: 25 - DNA, 3077 bp, Mus musculus
  CDS (59)..(2200)
ccacgcgtccggccagccccagggaccgcagcaatctcccaaactccccttccccaggatgacagaggagttaatcacccctgtgtactgcaccggggtgtcagcccaagtacagaagaagcgggacaaggagctgggcttgggccgccatgaaaacgccatcaagtacctgggccaggattatgaaacgcttcgggcaagatgcctgcagagtggggtcctcttccaagacgaggccttccctccggtttctcatagcctgggcttcaaggaactgggtcctcattcctctaaaacctatggcatcaaatggaagcggcctacggaactgatgtcaaacccccagttcatcgtggatggagccacccggacagacatctgccagggagcactgggggactgttggctcctggctgccattgcctccctcaccctcaacgagactattctgcaccgagtggttccctacggccagagctttcaggatggctacgctggcatctttcatttccagctgtggcagttcggggagtgggtcgacgtggtcatagatgacttgctgcccaccaaggacgggaagctggtgttcgtgcactctgcccagggcaacgagttctggagcgcactgctggagaaagcctatgctaaagtgaatggcagctatgaggctctttcgggaggctgcacctcagaggccttcgaggactttaccggtggggtcactgagtggtacgacctgcagaaggcccccagcgacctctaccagatcattctaaaggccctggaacgaggctccttgctgggctgctccattaatatctccgatatccgtgatttagaggctattacttttaagaacctggtgaggggccatgcgtactctgtgacgggcgccaagcaggtaacttaccagggccagcgggtgaacctaatccggatgcggaacccctggggtgaagtggagtggaaaggaccctggagtgacagctcctatgagtggaacaaagtggacccctatgaacgagagcagctgagggtcaagatggaggatggggagttctggatgtcgttccgagacttcatccgtgaattcaccaaactggaaatctgcaaccttacaccggacgcccttaagagcaggaccctccggaattggaataccacattttacgagggcacctggcgtcggggaagcaccgctggaggctgcaggaactacccagctaccttctgggtaaacccccagttcaagatccggttggaggaggtggatgacgcagacgactatgacaaccgggagtcgggctgcagcttcttgctggccctcatgcagaaacaccgccgcagggagcgtcgctttggccgggacatggagaccatcggttttgcagtgtaccaggtccctcgggagctggcgggtcagcctgtgcacttgaagcgtgatttcttcctggccaacgcttctcgggcgcagtcagagcacttcatcaaccttcgggaagtcagcaaccgtatccgcctgccgcccggggagtacatagtggtgccctccaccttcgagcccaacaaagaaggcgacttcctgctgcgcttcttctcagagaagaaggctgggacccaggaactagatgaccagatccaggccaacctccctgatgagaaagttctctctgaagaggagattgatgacaacttcaagaccttgttcagcaagttggcaggggatgacatggagatcagcgtcaaggagctacagaccatcttgaacaggatcattagcaaacacaaagacctgcgcactaatggcttcagcctggagtcgtgccgcagcatggtgaacctcatggatcgagatggcaacgggaagctgggtctggtggagttcaacatcctgtggaaccgcatccgaaattacctgaccatcttccggaagtttgacctggacaagtctggcagcatgagtgcctatgagatgaggatggccatcgaggctgcaggcttcaagcttaacaagaagctgcatgaactcataatcacccgctactcggagcccgacctggccgtggactttgacaactttgtgtgctgtcttgtgcggctggagaccatgttccggtttttcaaacttctggacacagacctggatggtgttgtgacctttgatctatttaagtggctccagctgactatgtttgcctgaagtggggaatcgctccgctccagttgcctttgttcttccattcttcctctgccaagccacgcctccccgccaagccacgcctccccgccgtgccactccagactacaccagcttctggtgccttcctcagaacaggaaggaaggcacccttgtcctcctgttccccttgtcctcctgtcctttcacctccccactacctctgttcgactgctctgggcagtcacggggccctccctgcccctgtagtcagactgggcaccctgacccctgctcagggctaagaccaggaagacagctccctgctcatccctgcctcggggctggggcaggggcaggggcaggggcaggggcaggcacccctgcccctccccctcgctgctactgtccggatgtgagtcttgcctaatctgtggaggctctagcccctgtcatcccctagagctgcagacttcataaccactagctagctccctgtctgcacccgggtgttgaaggcctcacctcactgtgggagccccacagccagactcaccttcctgtgccttctgtgcagcagccaagatccctctgcaaaccaaactacacctctcctagcctgtgtgcccaccaggaacttcctggccccgagggtgctcaggccctccccatccccgacccctctccctctctttttttgtatatatactggtcattttcaagggaatgcctgagggatgagtgtactggttatggaggaactgggggtggggtagggaggtcccgtgtttcagtgcagtgagaaaataaaatgaaaggctgtaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - protein, 713 aa, Mus musculus
MTEELITPVYCTGVSAQVQKKRDKELGLGRHENAIKYLGQDYETLRARCLQSGVLFQDEAFPPVSHSLGFKELGPHSSKTYGIKWKRPTELMSNPQFIVDGATRTDICQGALGDCWLLAAIASLTLNETILHRVVPYGQSFQDGYAGIFHFQLWQFGEWVDVVIDDLLPTKDGKLVFVHSAQGNEFWSALLEKAYAKVNGSYEALSGGCTSEAFEDFTGGVTEWYDLQKAPSDLYQIILKALERGSLLGCSINISDIRDLEAITFKNLVRGHAYSVTGAKQVTYQGQRVNLIRMRNPWGEVEWKGPWSDSSYEWNKVDPYEREQLRVKMEDGEFWMSFRDFIREFTKLEICNLTPDALKSRTLRNWNTTFYEGTWRRGSTAGGCRNYPATFWVNPQFKIRLEEVDDADDYDNRESGCSFLLALMQKHRRRERRFGRDMETIGFAVYQVPRELAGQPVHLKRDFFLANASRAQSEHFINLREVSNRIRLPPGEYIVVPSTFEPNKEGDFLLRFFSEKKAGTQELDDQIQANLPDEKVLSEEEIDDNFKTLFSKLAGDDMEISVKELQTILNRIISKHKDLRTNGFSLESCRSMVNLMDRDGNGKLGLVEFNILWNRIRNYLTIFRKFDLDKSGSMSAYEMRMAIEAAGFKLNKKLHELIITRYSEPDLAVDFDNFVCCLVRLETMFRFFKLLDTDLDGVVTFDLFKWLQLTMFA

SEQ ID NO: 27 - DNA, 2105 bp, Mus musculus
  CDS (418)..(1671)
gagccttcccggcgcgtgagccggatccggtggcaccgcggggaagagacaggaccgggcggtggcggcagagacagggggacgcacccggtgcagaagatccaataggagcacgccgccgcaacctctcccgcgcgctccggtcgccgactctacgccgatcgcccactccccgcaccttggactacaccgggaaaaagacgcagcctggcccgaactggggcggagatccttcttgcttcccaagagctcgggagggaagattctcccgccgccgagaaccccgccggactggaggaacccgtggcctggagagcgctggctgtgccagaccccagcctgatggatgtctggtgtggataatgagggaagaacgtgccttttacacccaagaggtgaccccggagcgtgccccggatgaccccacaactcggaacgatgcggctagcctgcatgttctcgtccatcctgctgtttggagctgcgggcctgctcctcttcatcagcctccaggaccctatagagctcagcccccagcaagttccaggtataaagttcagcatcaggccccagcaaccccagcatgatagccacttgaggatatccacagaaaagggcacacgagattcacccagcgggtcgccaagaggcctccagctgcaagcgcctgaccaacctcgacctcacccgaaggcagcgggatctcctttgcgcctccggcagcgcaggcggagactgctcatcaaaaagatgccagccgcagggactaaccaaggcaacaactcgtccgaaacctttatccagccgagaccccgcaccatggacagtcgttgggtcagcctgcaccagacccaacaggagcgcaagcgtgtgatgcgagaagcctgcgctaaatacagggccagcagcagccgcagagctgtcactccccgccacgtctcccgcatcttcgtggaggaccgccaccgtgtactgtactgtgaagtacccaaggcaggctgctccaactggaagagggtgctcatggtgctggcagggttagcctcatccacggcagatatccaacacaacaccgtccactatggcagcgcccttaagcgcctggatacttttgaccggcagggcatagtgcaccgcctcagtacctacaccaagatgctctttgtccgggaaccctttgagcggctggtctctgctttccgagacaagtttgagcatcctaacagctactatcatcctgtctttggcaaggctatcctggcccggtaccgcgccaacgcctcgcgggaggcactgcggactggctccggtgtgcagttccccgagttcgtccagtacctgttggatgtccaccggcccgtgggcatggacatccactgggaccatgttagccggctgtgcagcccctgcctcatcgactatgactttgtgggcaagttcgagagcatggaagacgatgccaacttcttcctgcgtctcatccatgcgcccgggaacctgactttcccgaggttcaaggacaggcactccgaggaggcgcggaccacatcgagaatcacccatcagtacttcgctcagctctcctcgctgcagcgacagcgaacctacgacttctactacatggattacctgatgttcaactactccaaacctttctcggacctgtactgagggcggggcctgctggtcaggggcggggtctgccggtcatgcccactcacctgcgcataggcggcctccggggactgagctctgaggatgtgaggccttgtggctgtggccctagggtgggccacagaggcccagacaatggaccttgacccttgtcccacacccatttcctcattgggttggctgagtttgagacggagcacgactcggatggatgctttaagaactcagctgagctatgctgagctatgctgtccagggaagcctgagacccagaagagggccccagcgtcgagggatgtcctacatccccttatcctttgccttgtaccaaaccacgtggtttgctgcttttctatgacccagggtcatctgaataaagcacatggttttcagagcaaaaaaaaaaaaaaaaaaaaaaaaaaaaaaaaaaa

SEQ ID NO: 28 - protein, 417 aa, Mus musculus
MTPQLGTMRLACMFSSILLFGAAGLLLFISLQDPIELSPQQVPGIKFSIRPQQPQHDSHLRISTEKGTRDSPSGSPRGLQLQAPDQPRPHPKAAGSPLRLRQRRRRLLIKKMPAAGTNQGNNSSETFIQPRPRTMDSRWVSLHQTQQERKRVMREACAKYRASSSRRAVTPRHVSRIFVEDRHRVLYCEVPKAGCSNWKRVLMVLAGLASSTADIQHNTVHYGSALKRLDTFDRQGIVHRLSTYTKMLFVREPFERLVSAFRDKFEHPNSYYHPVFGKAILARYRANASREALRTGSGVQFPEFVQYLLDVHRPVGMDIHWDHVSRLCSPCLIDYDFVGKFESMEDDANFFLRLIHAPGNLTFPRFKDRHSEEARTTSRITHQYFAQLSSLQRQRTYDFYYMDYLMFNYSKPFSDLY

SEQ ID NO: 29 - DNA, 1369 bp, Mus musculus
  CDS (40)..(1281)
attcagacatgaagagagacacagtggtctgaagtggtcatgaaagccaaacaagtcttcttttctgtcctgctgtttgggacagcagggcttctgctcttcatgtacttgcaagcatggattgaagaacatcatacagggaaaatagagaagaaaagggatcagaaaggagtatcggtgactacgggaaaaatccagaaacagatcacgaatcagaactctgaggttcacatgcctgaagatctgaagaagaaagggggagatctgctcaacctagggagtccaacaagggttttaaggaagatcagccattcacaaagggagaacggagcttacagatcaactgaagcacatcaaggagctaaaattgaagtttttcagaaacccatccagatggactggccactggtcactcagcccttaaacaaaagtttggtccaaggcaacaaatggaagaaagcagatgcaacccaagagaagcgtcggtcattccttcatgagttttgcaagaaatatggtagagtaaatgatcccaagttcaacctttttcatatagtatctaggatatatgtagaagacaaacacaaaatcctgtactgtgaagtaccaaaagctggctgctctaattggaaaagaattctgatggtcctaaatggattggcttcctctgcatacaatatctcccatgatactgtgcactatgggaagcatctgaaaacactggatagttttgacttaaaaggagtacacatgcgtttgaatacatataccaaagctgtgtttgttagagatcccatggaaagattagtctccgcatttagggataaatttgagcatcccaatagttactaccatccggtgtttggaaaggcaattatcaagaaatatcgaccaaatgcctctgcagaagcattaaataatggatctggagtcaaattcaaagaattcgcctactatttgctggatgctcaccgtccagtaggaatggatattcactgggaaagagtcagcaaactgtgttatccgtgtttgatcaactatgactttgtagggaagtttgagaccttaggagaggatgccaattactttctacagttgattggtgctccaaaagagttgacatttccaaactttaaggataggcactcctctgatgaaagaaccaatgcccacgtggtaaggcagtatttaaaggacctgagcacagccgaaagacagctcatctatgacttctatcacttggactatttgatgtttaattacacaactccacatttgtaatttgcattcatttttctaaagccctacatagatttaatgatgatggcctcaaataagctactgtaattgtcctacaattctctgtatg

SEQ ID NO: 30 - protein, 413 aa, Mus musculus
MKAKQVFFSVLLFGTAGLLLFMYLQAWIEEHHTGKIEKKRDQKGVSVTTGKIQKQITNQNSEVHMPEDLKKKGGDLLNLGSPTRVLRKISHSQRENGAYRSTEAHQGAKIEVFQKPIQMDWPLVTQPLNKSLVQGNKWKKADATQEKRRSFLHEFCKKYGRVNDPKFNLFHIVSRIYVEDKHKILYCEVPKAGCSNWKRILMVLNGLASSAYNISHDTVHYGKHLKTLDSFDLKGVHMRLNTYTKAVFVRDPMERLVSAFRDKFEHPNSYYHPVFGKAIIKKYRPNASAEALNNGSGVKFKEFAYYLLDAHRPVGMDIHWERVSKLCYPCLINYDFVGKFETLGEDANYFLQLIGAPKELTFPNFKDRHSSDERTNAHVVRQYLKDLSTAERQLIYDFYHLDYLMFNYTTPHL

SEQ ID NO: 31 - DNA, 4264 bp, Mus musculus
  CDS (561)..(2246)
ggagtcaggttgacgcgccctgggtgcggggtgctaggcgcggggctgggcgcgcgctcccgcggagtcggagctgcagaggcttccgagatcgtcttgggcttttcaggtcacctagcattggtatgaatctgctcctcaagaagagaaaatattctgaaggcatgtccgtttacctgtcaacgaggaacccataatgtcttctcatctccagtgtggctccatgctctctcctgatgctactccaactttctttggtgccatgggaaaaacctgaagcacgagcagagtcacctccaaatctgtgctcacttcattttgttgcatggctggtgcggaacagactattggaatttgctcttgtgtgttgcccttcgtacctgaaccaaagactgtgcctcattctctagtgcattgctgcctgctgtcttcctcgttcatttctgttgcttgtgccttcctactgtcttctgtcatcacaggacacccttaaagtttctcactggactcctcgagggctggcccatggctctctaagccagctgtccaaggtctctaccatgaggcattgcattaattgctgcgtccagctgttccccgaagacacacacaaacagcaagttgcctgccaaggaggcccccatcacagtcatcaggcgtgccccacttgcaaaggagaaaacaaaattctgtttcgtgtggacagtaagcagatgaacttgcttgctgttctcgaagtgaggactgagggcaatgaaaactggggtgggtttttgcgcttcaggaaggggaagcgatgtagcctggtctttggattgataataatgaccttggtgatggcttcttacatcctttccggggctcatcaggaacttctgatttcctcccctttccattatgggggctttcccagcaaccccagcgtgatggatggtgagaaccccagtgatgtgaaagagcatcactaccagccttctgtaaataacatctcctatgtaaaggactatccgagcattaaactgattatcgacagcattgctgccaggattgagttcacaaccaggcagctcccggacttacaagatctcaagagacaagagttgcatatgttttctgtaatccccagcaaattcctcccgactagcaagagcccttgttggtatgaggagttctcgggcaggaacaccactgacccctacctgaccaattcctacgtgctctactccaagcggttccgctctacctttgacgctctgcgcaaggttttctggggccacctgtcccacgtgcagggcaagcacttccgcctgcgctgcctgccccacttctacatcattgggcagcccaagtgtggaaccactgacctgtacgaccgcctccggctgcatccagaagtgaaattctcagccatcaaggagccgcactggtggacccggaagcgctttggaattgtccgcctgcgggacggactacgagaccgctaccctgtggaagattacctggacctctttgacttggctgcacaccagatccatcaaggactgcaggctgcctctgcagagcagcccagcaagatgaataagatcattattggagaggccagcgcctctacaatgtgggataacaatgcctggaccttcttctatgacaacagcacagacggcgagccaccattcctgacccaagacttcatccatgcctttcaaccagaagccaagctcattgttatgctcagggatcctgtggagaggttgtactcagactatctctacttcgcaagttccaataaatctgcagatgacttccacgagaaagtgacggaggctctgcagctgtttgaaaattgcatgctggattattcactgcgcgcctgcgtctacaacaacaccctgaacaatgccatgcccgtgaggctccaggttggcctgtatgctgtgtacctcctggattggctgactgtcttcagtaaggagcagttcctcattctccgtctggaagaccacgcatccaatgtcaagtataccatgcacaaggtcttccagtttctaaacctggggcctctgagtgagaagcaggaagctctgatgaccaagagccctgcatccaacacacggcgtcctgaggatcgtagtctgggacccatgtggcccatcacccagaagattctgcgtgaattctatgggcctttcaacacaaggctggcacaggtcttggatgacgaagcatttgcctggaagacaacgtgagagctgagtcccttctgcagaagctgggcccactgactctgtcattgccatgactttaaaagtctccaggcggagaactgtcacccacagcgtggagaaagccgctggagccccacccaatcctcttctcatgtccaaggcacctggtgtgggaactattttaaagctttccttgccatgttcagcagctctgctcttgattgcaaggctcgtccacctgaatccttggaaacgacactgggggtaggcttctgtctgttcccagagtgccttggatctcaggagggcagtggatctctggcacccagtgggtaggaagaggaagctacgggaggccatagaccagccttgccttccttcctccaggagagtttgtctgggtgtagggttggacagctctgggatccgattaaggccatgttctggggctgcccctcacgtgttatatcccagcaggccttgcataaatcgacaaccccatgtggacagagaacagagctgcttggagaatggaggctctttcgtttttgttcgtattcccaagtcagcccttcggtgacatcagaaatgagccctgttccctttgctgtgctcagaagaaactcagaagtcagcttttggcagtaggctcgtctctctgacctattctgcccatacactttgtttatgcccagcgtagagaaggagcgttttaaaacaaactgcttctcgcatttgactggccctgcctctcttttctgctcccttggatgttttccttaaggacttcagtttatatttggaaagggaagttttgctgttaatgccagaacagaacacctcaaggcatcgaacagtggaaaaggaaggaagtgaagccctccccatttgtgggggtcactggagagtgtggggatgtttttggttctggctggttacaggctcccccctgtcttcctgttggaggaatctttatccacattctctgtcatgtgcagccaaagttctgtaactcactaagggtgagtctcaagacacccttagaatgtcctcagaaacacatctcacaaactaagctcgggccacatgtggtgtcactaggagatagctttgatttttatgaacctttccaggcctgctagcagcttggttcgcaaaggatatgctgaaagtcttgtcattttaaaaaaaaaaaaagaccccagggaataccagagagaacaacttaaacccacttggcacacaggacaaggtggggatgtcacaaagcttttcattggagcgttgccaagggatgctgcctgtgaagctgtgttctcaggtcaggatcctgttcaccagcgtcagaaagtgtacacaccacatgcccgactgtcacttggacgtgtgagaaaagctgggctttggcagcaggaggctgtgcagttgctcgactgtgtgagttctgctgcggtccacccttgttggagatgggactctcagctcattggaaggttccgggagagctctgggcatcagcatggactgggctcgttctgctactgaaggggctgtgtgtcatgtggcctgtgttccaggacaattctcccttctctcacggtggccacttggctcggcagccagtgggaaaatctagcctgagtacaaggatgctttgggaagaaatgacgagagagagagagagagagagagagagagagagagagagagagagagcgagagagagcgcaagctcaagcgcaagcgtgtgagaaagagcacgagagagtatttatttggaataccgtgtgtgtgtgtgtgtgtgtgtgtgtgtgtgtgtgtgtgtgtgtgtgtgcgtgcgcgcgcgagtgtgcgcgcgaacactcgtgcttatactgagcagggccctctcagctgtcttgttgatggggcttctcctccattagagacatgatgcctttatgaattaatttctccaacccttcgcctgtcatttccaggacatatcttactgccccacaccaaccacgactctaagagattttttctgacgcactccc

SEQ ID NO: 32 - protein, 561 aa, Mus musculus
MRHCINCCVQLFPEDTHKQQVACQGGPHHSHQACPTCKGENKILFRVDSKQMNLLAVLEVRTEGNENWGGFLRFRKGKRCSLVFGLIIMTLVMASYILSGAHQELLISSPFHYGGFPSNPSVMDGENPSDVKEHHYQPSVNNISYVKDYPSIKLIIDSIAARIEFTTRQLPDLQDLKRQELHMFSVIPSKFLPTSKSPCWYEEFSGRNTTDPYLTNSYVLYSKRFRSTFDALRKVFWGHLSHVQGKHFRLRCLPHFYIIGQPKCGTTDLYDRLRLHPEVKFSAIKEPHWWTRKRFGIVRLRDGLRDRYPVEDYLDLFDLAAHQIHQGLQAASAEQPSKMNKIIIGEASASTMWDNNAWTFFYDNSTDGEPPFLTQDFIHAFQPEAKLIVMLRDPVERLYSDYLYFASSNKSADDFHEKVTEALQLFENCMLDYSLRACVYNNTLNNAMPVRLQVGLYAVYLLDWLTVFSKEQFLILRLEDHASNVKYTMHKVFQFLNLGPLSEKQEALMTKSPASNTRRPEDRSLGPMWPITQKILREFYGPFNTRLAQVLDDEAFAWKTT

SEQ ID NO: 33 - DNA, 1718 bp, Mus musculus
  CDS (374)..(1564)
acaaagcccggggggtccacgctggcgctggaggcgagccgggagcgagcccggggcgcggcgggacttggggcgcagggctgctacgggacgcggggctgctagcaggacgcgcgaccaggcgtgggttgcgatcgccacccccacggcggggcccgggacattttgggccctttggggccgcagcgagcggtggggaccaagacgagcagagtaccctgagcccggcaacttcggcccctccccgcccccacccggctgccctcagcgcgtccctctccatgtgcagacagccggctaggcgctccggctggcggcggatgggtgacctcttcctggccggggcgggctgttcgaggcggcggagccggcgatgaagaagaagcagcagcagcatcccggcggcggcacggatccctggccccatggggcccctgtggggggcgcccctccgtgcctgggcagctgcaagcgccggatcccgctgctgcctttcctgcgcttctccctccgggactacggtttctgcatggctaccctggtggtcttctgcctgggctccctcttctaccagctcagcgggggaccccctcgcttcctgctcgacctgcggcagtatttgggaaattccacttacttggatgaccatggaccacctcctagtaaggtccttcccttccccagccaggtggtgtacaacagagttggcaagtgtggcagtcgtaccgtggtcttgcttctgagaatcttgtcagagaagcacggcttcaatttggtcacatcagacattcacaacaagacccggcttactaaaaatgaacaaatggaactgattaagaatataagtaccgccgaacagccctatttattcacgagacatgtccacttcctcaacttctccaggtttggaggagaccagcctgtctacatcaacatcatcagagaccccgtcagtagatttctgtctaactatttcttccgtcgctttggagattggagaggggaacagaatcacatgatccgcacccccagcatgaggcaggaggagcgctacctggacatcaatgagtgcattctggaaaactaccctgagtgttccaaccccaggctgttttacatcatcccatatttctgtggacagcaccccagatgtagggagcccggcgagtgggcgctggagcgggccaagctgaacgtgaacgaaaacttcctgctcgtggggatcctggaggagctggaagatgtgctgctcttactggaaagatttttacctcattacttcaagggtgtgctgagcatctacaaggacccagagcacaggaaagcttggcaacatgactgtgaccgtgaggaagactgtcccctcccccgaggcggtgcagattctctaccagcgaatgagatacgagtacgagttctaccactacgtcagagaacaattccacctgctcaagcgcaagcttggactgaagtctcgcgtgagcggacctcccgtgagaccacagttcttcatccccacgcccctggagacggaggagccaatcgacgacgaggagcaagatgacgaaaaatggctagaagatatttataagaggtgatacctgcgccagattgtggcttctgtggcctcatccccccttttacaaagttctttggggaagagaaattctgaagggactttcattaatgctcgagtgcattaaaaagaacaaaacatt

SEQ ID NO: 34 - protein, 396 aa, Mus musculus
MKKKQQQHPGGGTDPWPHGAPVGGAPPCLGSCKRRIPLLPFLRFSLRDYGFCMATLVVFCLGSLFYQLSGGPPRFLLDLRQYLGNSTYLDDHGPPPSKVLPFPSQVVYNRVGKCGSRTVVLLLRILSEKHGFNLVTSDIHNKTRLTKNEQMELIKNISTAEQPYLFTRHVHFLNFSRFGGDQPVYINIIRDPVSRFLSNYFFRRFGDWRGEQNHMIRTPSMRQEERYLDINECILENYPECSNPRLFYIIPYFCGQHPRCREPGEWALERAKLNVNENFLLVGILEELEDVLLLLERFLPHYFKGVLSIYKDPEHRKAWQHDCDREEDCPLPRGGADSLPANEIRVRVLPLRQRTIPPAQAQAWTEVSRERTSRETTVLHPHAPGDGGANRRRGAR

SEQ ID NO: 35 - DNA, 5091 bp, Mus musculus
  CDS (318)..(1595)
ggcggcacccaaacagctaccccgtgcggagacccaaactttctactgccacttggagtctcgcggccgccgcctccgccccgcgcgtccggggcctgcccgtcagtccgtcggtccgcgtggagcagctcgggcgccgccgtgctcccgatccccgcagctgcagcgccgcagtcctggcccggacgcccgggcaagttctccagagttaaaagcgaagttcgggcgaggcgcgggccgagctgcctctgagcgcccccggcgtccccagtgcgcccagccccgccgggggcgccggtgacccttcggtgccagccatgtcgtccatcctgcccttcacccccccgatcgtgaagcgcctgctgggttggaagaagggcgagcagaacgggcaggaggagaagtggtgcgagaaggcggtcaagagcttggtgaagaagctcaagaagacggggcagttggacgagctggagaaggccatcaccacgcagaacgtgaacaccaagtgcattaccatccccaggtcactggatggtcggctgcaggtgtcccatcggaaggggctccctcacgttatctactgccgcctgtggcgatggcccgacctgcacagccaccatgaattacgggccatggagctctgtgagtttgccttcaacatgaagaaggatgaagtgtgtgtaaatccttaccactatcagagagtagagacgccagttctacctccagtgttggtgccacgccacaccgagatcccggccgagttccccccactggatgactacagccattccattcccgagaacactaacttccctgctggcattgagccccagagcaatattccagaaaccccacctcctggctacctgagtgaagatggagaaaccagtgaccaccagatgaaccacagcatggacgcaggttctccaaacctctccccgaatccgatgtccccagcacacaataacttggacctacagccagtcacctactgtgagccggccttctggtgctccatctcctactacgagctgaaccagcgagttggggagacattccacgcctcacagccatccatgacagtagatggcttcactgacccctccaactcggagcgcttctgcctgggcctactgtccaatgtcaaccggaatgcagccgtggaacttacaaggcgacacattgggagaggtgtgcggctctactacatcggaggggaggtctttgcggagtgcctcagtgacagtgctattttcgtccagtctcccaactgcaaccagcgctatggctggcacccggccactgtctgcaagatcccaccaggctgcaacctgaagatcttcaacaaccaggaatttgctgccctcctagctcagtctgtcaaccagggctttgaggctgtctaccagctgacgcgcatgtgcaccatccgtatgagcttcgtcaaaggctggggagcagagtacaggagacagacagtgaccagcaccccctgctggattgagctacacctgaatggacccttgcagtggcttgtcaaggtcctcacccagatgggttccccgagcatccgctgttccagtgtgtcttagagacactaggagtaaagggagcgggttggggagggcgggcttggggaaaatgaccttggaagagaactccatccaacttggtcttgtcaaagaacaccgattccactcaactaaggcaccagcctgtttctgagaccacagaagaaaaccccagggatggatttatgaacagctgtgtctgctacatacacgtgcccctgtctgaaggccaagtgatggcttctgttctggtggcttgaactaacaggtggtgtatcgccacctgactccttgtttaatgacagaggtctgggatgtcacagtccaaaaggaaagtgcctttctccatggctggagtatggagtttacctttggagaagttgtaatggagcatgccctgtcccaccactctcagagagggtgtacctgtcaaactggatggcctacataggtactcccccctacccctaggatgcagagagacgggaacacgccggagggtagagctggggagaacccattcttccttggaaggatccgctgaaggtcagcgtataggtgatgtacagttcctaatatcacatcagtctcagagtgttcacaggaagcagcaagggcactcgtggagtatgtgtcctgggtgaggtggcaccacaccgaatgaatgcatctctgggagctggcaccacaaccctgatgtataggctgtgttggtttatggagacaagttgcatcaatgaattcacctagcataggctctttgaaatgtcctctgtttgataaaaaacaatcctgggtacgtatgttggctggaaaaccacaatggaccctgccactgcttcttgccctgaggtttggaagctgagagttatagaagccaattcaccaggaggtaagacatcccaggctgacatgggcaaatgaaaagggctattaaaatttttttacaccttggaaaattgcaggcttggtgcagagcgctctgtcatcttcaccctgggatgtaggattacctagctatggtaaaggattgccacagcaaactgtgacactgtgtaatgagcactgttcccagcggcaattacagagaaaacgagtgtaaattatttcaactggaaaaaagtccctttcttggctgttttagaacagggtacacaggatcgccacctgcaactggtactcgcttcttggctgctgcttctgttgtgaaaagacgagcccatgtttctgcatggatttcccatggaagtcctgtcctgctacagaggggaagaaagtgtaccctccaatgtgataaatcttctgatgcccccagactcttggagcacatcctggtgcccctcctgcaggagcctgtggcatatttccagctgggcatgctgatcctccttgagacacagatgcctgtgtgagtctccgttgatacaattctgaacccctcaggttctctgaaagggcacagaccatgggcgtgaacattgtgccgtacctgagttggtctgtggactgctgcttcagacacacgagtcctcggaactgcctggctgcctgtcacgcatgctctgagtcagaacacaccaacgctctgctgtggctcctagggaagcattcatggtcctctgttatcagcaggggtttatgtcacttgctgtccggtttcctaggggcttcctgtgccccttccccagctatcctccaggtggctagggacagtctattctgctgcaactggaaagtagagggaaccggcactgctcagagcagatggcggcttctggaggcacacagtgggagtacaccccttcatgttattggccagttgctggagaatgctgtaggagaaaattctaggcaggtctactcttggcatccctgagagtcaaaggcttggagtctaggaaaggtcacaccatgatggagaacacaggtcatttgggtacgtgtaatcaaagtgccctcccaaatcagttctccttttcgtatgaacagcatctctacttttaaagaggagttgaggatcgagaagatgacagtgcagcagtgggtgtggcctgactacatgtgctgttccagccctgggtgcccactgacaccgacccccaggcagaggcctttgtcttcagcactcctgagaagttggctctttaccccttctcctctgctgcccctccttcctgctggttcaggtagccccagccacgtgggttagagtcctgtgctggcctgccatggcagctggctaccttccagaccaactgtagagatacctggcattttgaagaaagcctagactggagagcagggcctctcttgggaaggacacaaggcgggcaaggctgctggcagacttctccactgacctgagtgtgctttttttttcccctaaatgtattgcatcaagcctcagtgcttatggagtgcagtggtcttcatcttccccaacttgcttctcagagctgggatgtattccagagcctgatgtttttattcaaaccacagaaaagttttctttaagtagcctgtgcagtcatgcatgtgcctgagttgtctggagcagaggcaaacatctgacttcattcttagccccaagctgccatttctgagtccttgagaggctgagaaggctctagctttgtactgtattcttactgtgtgactagatgcgtgagcgctttacattagaaggaacctggttagagctcgctcctcctgtctttgtgtggcatttgtgttccattaccggcccctttaagtaacggccttcacagcaccttcccagtgggtagaaagccacacaccaggatgtgggtcaaccatgaagatgtggcattgcagacgggggaacatgtggatgcatggctatcgccctgaacagtccctgcagctacttgtgttaacacagaactgatgtttagcattctgccgctttcgtatttatgtaacaattccttaaagccattcaaatggctaactatttaatttctttaggacagttgtaaaggtctctctcctgaggacaatgacttggaagaactggggcacagccagtcccagacactggtggaggctgcagtgactttttttggctcaagatccacaagcattagagtagactgggccaacaagtcaacaagtggtggcgtgtgcaaacgggctgccctagtcaagcccagtcccttcaacagtatgtctgatgcaccacaggccctccctactggaagtgggaacttcaaatggaaattggagccatcttttatcccagaagcctttgctgctgccagggcaagtgggctggtgtggactcttgtttaggaggctgaggttcttgtcactccttagccagccaggcctttagtgtctttgtaaaaagcatgtatttatagtgttttagatttttctaacttttgtatcttacagcattgtaccccattgttaaagagccgtgtcccctcttcttataaacgcccttttaataaacttttcaccgtaaagctcctgagacaggagcacagtctgt

SEQ ID NO: 36 - protein, 425 aa, Mus musculus
MSSILPFTPPIVKRLLGWKKGEQNGQEEKWCEKAVKSLVKKLKKTGQLDELEKAITTQNVNTKCITIPRSLDGRLQVSHRKGLPHVIYCRLWRWPDLHSHHELRAMELCEFAFNMKKDEVCVNPYHYQRVETPVLPPVLVPRHTEIPAEFPPLDDYSHSIPENTNFPAGIEPQSNIPETPPPGYLSEDGETSDHQMNHSMDAGSPNLSPNPMSPAHNNLDLQPVTYCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLVKVLTQMGSPSIRCSSVS

SEQ ID NO: 37 - DNA, 1983 bp, Mus musculus
  CDS (80)..(1171)
tggagctggtcggttatggttggaactttccctgcccttcgttgactgagtgaaccagccagaaatacattcccaggaaatggcctctaaatcttggctgaattttttagtcttcctctgtggatcagcaatagggttttttttatgttctcaactcttgagtattttgttgcgagaagaggctgccattcagcctaacatgcttcacaatgaccctcatgcaaggcattcagatgacaatggacacagtcacctcaaaggacagatgaacttcaatgcagattccagccaacataaagatgagaacatagacgttgctgagaacctctatcagaaagttaaaattctttgttgggttatgacaagtcctcaaaatctagagaaaaaggccaaacatgtcaaagctacgtgggcccagcgttgtaataaagtgttatttatgagttcggaagaaaatcaggacttccctactgtgggattgaaaaccaaagaaggcagagagcaactatattggaaaacaattaaagctttccagtatgtacatgaccattatttagaagatgctgactggtttatgaaagcagatgacgacacatacgtcattgtggacaacctgagatggcttctatcaaagtatgaccctgaacaacccatttactttgggcgaagatttaagccctatgtgaagcagggatacatgagcggaggagcgggctatgtcctaagcaaggaagccttgagaagatttgttaatgcatttaaaacagaaaagtgtacacatagttcctccatcgaagacttagctctgggaaggtgcatggaaattataaatgtagaagctggagattccagagataccattgggaaagaaaccttccatccatttgtaccagaacaccacttaatcaaaggttatctaccaaaaacattttggtactggaattacaactattatcctcccatagagggtcctggatgctgttctgatattgcagtttcttttcactatgttgatgggacaactatgtatgaattagaatacctcgtttatcgtcttcgtccatatggttgtttatatagatatcaacctgccttacctgagaatatactgaaagaaattaatcaagtaaacagaaaggaagatacaaaaataaaattaggcaacccctgaaagcagaacataagtggtctacattgcactgaaggactcttgcctttctacggaaccctgaaatcccagtgaggaactcacctgaagtgaacattccatatagaaatctttcaaatggatgactataaactgaagcatttaaagagctgtgaagtttgctaaaacgtgtcttgatacagtaatatataaatataaataaatatatatgaagaatttgtgtttttaaatggtgacagaagagctagaaagactctgtttctgactgttcccaacagtgtatgttgttatcactacagttggagtggttagtttataatcaccttctgcaccacaccagttagaacacaatgctgaacagatctgccttaaagaaagaaagaaagaactgttgctcagtgttgtttctcagctgatatgtgtatgctgcaaaccagccttccagttgccgaatgaaaactttcctgtgattatattatttataattggcagtgcttcctcaagaagaataagaagggcacttaccccaaagtgcagtaattaaccacatttccaattattccgagccttgactccattagatacttagttctaaagataatcaacccgataccaagtccccattgtcccctttccaaatgtttgaccctgaataccatttttcactattaatgctaatttactcaaagcattggcagcattctgtaaatcatgcattttcaaatatgttgttccccgattcacccattgttaattgtaggtaacaattaaatttaatgctgtttggacaagcaaaaaaaaaaaaaaaaaaaaaa

SEQ ID NO: 38 - protein, 363 aa, Mus musculus
MASKSWLNFLVFLCGSAIGFFLCSQLLSILLREEAAIQPNMLHNDPHARHSDDNGHSHLKGQMNFNADSSQHKDENIDVAENLYQKVKILCWVMTSPQNLEKKAKHVKATWAQRCNKVLFMSSEENQDFPTVGLKTKEGREQLYWKTIKAFQYVHDHYLEDADWFMKADDDTYVIVDNLRWLLSKYDPEQPIYFGRRFKPYVKQGYMSGGAGYVLSKEALRRFVNAFKTEKCTHSSSIEDLALGRCMEIINVEAGDSRDTIGKETFHPFVPEHHLIKGYLPKTFWYWNYNYYPPIEGPGCCSDIAVSFHYVDGTTMYELEYLVYRLRPYGCLYRYQPALPENILKEINQVNRKEDTKIKLGNP

SEQ ID NO: 39 - DNA, 4263 bp, Mus musculus
  CDS (602)..(1576)
ccgagaaggagagagcggcacaggtttctgccgcggcgctcgaacgggagcttagggacgcagcatcctcggtgtccacccgggatcgcagctgcaccggccgacatgtgacggaggaaagccctcagcattcccggcaaccgccttcaaaggggtcccggaacgctcgcccaggccagcagctggatcctcttagccctcagcgacgcgggacaggagcggcaaggtacaacgcctggtacgttccaggctcgcactcgggaaaagagcgcagcggggaccgccgcctggcttgcactcggcacaactccgggacctgcggagtgcggctggaccgtgtccagattcccgccgacttcatccagagtgacccaagaccctagccggtcctcctgcaccccctggagttctagcttgccctgcatctgcgccgaccccacaacgcccaggtccccgcaacgccccagtccggaaggggagcgcgcttgcggcttctccctgattgcagacgctgccgctgtcctaactgcacacagaggtcccctacccggagcctagtccttgcagggggtggtgtccgagacgctggggagcgcgcaccatgaagtccgcgctgtgcagccgcttcttcatcctcctgccctggatcctgattgtcatcatcatgttggacgtggacccccgcaggcccgcgccccaactcacttcccgtccctacttctctccccatgcagtgggctgcgggggctcccgagtcccgctccgacggagtagccccgggcgtgacgctgccgagaagcggaacgagtctcggcctcagctgcagccggagccgcgcttgcccaccatctatgccatcacacccacctacagtcgcccggtgcagaaagccgagctcactcgcctggccaacaccttccgccaggtggcgcagttgcactggatcctggtggaggaccgggcgacgcgcagcgagctggtgagcagcttcctagctcgggccggcctgcccaacacgcacctgcatgtgcccacgccgcggcgctacaagcgaccgtggctgccgcgcgccaccgaacagcgcaacgcgggcctcgcctggctgcgccagaggcatcagcatcagagcgcgcaacccggcgtgctcttcttcgccgacgatgacaacacgtacagtctggagctcttccaggagatgaggaccactcgcaaggtttctgtctggcctgtgggactagttggtgggcgacgctatgaacgtccgttggtaaaaaatggcaaagttgttggctggtacaccggatggagagaagacaggccttttgccattgacatggctgggtttgctgtgagtctgcaagtcatcttgtccaatccgaaagctgtatttaagcgccgtggatcccagccagggatgcaagaatctgactttctaaagcagataacaacagttgaagaactggaaccaaaagctagtaactgcaccaaggttctcgtgtggcacactcggacagagaaggtcaatctagccaatgagccaaagtatcacctggacacagtgaacattgaggtgtagccgccaggaccagcaggtgaggacaggaagtctggagcgtgggctgctgcccattcaggtactgttcctttgccttcagcctttccttaccgccgatggacggccgtttcgacagctacctggatgctacaaaatgtgttgtcttcatttgtctgcatgtgttttctgaactggaagagttttgtagagtgaagccacatgtcactgaaaaggacaacgaagtatctcacactgctccctccctccctccctccctccctccatccctccctcccacaagccatctcaatactgctagaatgacaggttttagaagctgatgaggagtactctgtcaattaaatattcccagttaaattcttggcttcacaacatgaactatttggaaagctgatggcagtgtacactgtaggaaataaaacccacaaataaaaaggtgtgtggattcatctgtttaatatagggaaataacattttgtcaatactaggcaccataaaatgtaaacacaattactgtcataaacctggatataccttcaaggattaaaagtggctttgttttagttaccctgtgtgcatatagtgcagaaaaaggtcttcatattagcactatgtacatgagaagaggcccacattacaagaggcaatggaaatttgaattctgaaaccttcattgagttttacagtagcatccaggtttatctccgttcactaaccacgttccctgttaagcacatcataacaacagcacagtggagtgagtgatgaataaaagaatttgatacactagaaaacagtgctcagtgatacatttacattctatttatatgattaaacatttgatcatacagtacctttctacaggattactggctaatttgggggtggggtttatactgttagaggtattactaacatgataactacttcccttatatgcaaacgttagagctataattttgcaaataaaactgattatgcaagttaactgagcagcttcccaatgtggcttaggacatcatggggagtatgagcaaagctgccctccacacagatggtttctcaacctgctttcacatccaatttaatcagcacagaccaacacggtcaatcaggtcattcttaatgtgagcaaatgggggaaagaaaaaatacgttatgtacatgaataaatgggaactagatgcctttgcagcgaggaatgttggggtcggttctggatggaactctgtgacagcttcttacttccacagctgtgtgctcagcgtctgccccgatgagcttccagaccaccctgctgtcgtgctgggaggctggccaaaacctgcactagcgttagcactactgaggttcatggcagccatctgctgattcatctgcgaaacacaaaaggcttggtttacaaggaggaccctcttttcactcctgactccttagctacagccagacacttcatcagatgtgaaaattagtccagacactggatgtggactacactgaagtctttctactgaaatcagttacttttttcttacttttttctgagttaggtgttacttataaagtaatccataattagttcactttctagattaaccacaactgaaaacacatcaaagtaatctttgagtttatatctgagaccatcactcttctgtgactccaagactgagacattttagaaagtaaaacatctaaaattaccctaacatttacaaaatactcatacctctctattaacatcctaaatgaccttaatatacttcctaaacattaaaaaggtcatatagcatatgttttaggtttatgttataataacataaacagctaaaattaaatttcattatatgctatacaataaacaactacatgtacttttctttcaggctaaaatccagtatatttatcaggctcttagcacagtagttctcccactacccagtttggtgacggaacaagagatgcaggatttacctaaaatagtttcaaacctatgttcaacattatctgtgttctttgtgcaaattttttttaaaacccaagacataataagcacacatgtgtctacagacaggtagcttccatcttcccttctgctcactcttgtggccttgctgttcttcactggctctcctgtactctaacctgagtcatgtgagcctgagcatcctggtcacagaaaagctgttcctagtatctgacatcattcccgaggtttccaatgcataagcagtgtgtacgtatatacctataaaccgcagctgtcacagcacacgagcatttgtaagatacacggtgattctgtgaatgttagttagcagacattgttctgaaggtaaactaggggtcttagtggtcctggttcctgtgagatgctcggtggaccatcatgcaggaatgggaatgggaccatgagcactaagtcaaagcctctgatgtggggtaacagcagaaatgttactctaacctaagccagacagacatggaatttatcagtgaatacctgagggaagcagggtttacattctcaccatggggattcctgagccaatggatgttcctcatacatcaacaggcagtaaccacatgacagcggcagccttgatccctcataaaactgtgctttgaaaatatgaagctggatttctatctcactgataagaattaataaaaaaaaaaaaaaaaaa

SEQ ID NO: 40 - protein, 324 aa, Mus musculus
MKSALCSRFFILLPWILIVIIMLDVDPRRPAPQLTSRPYFSPHAVGCGGSRVPLRRSSPGRDAAEKRNESRPQLQPEPRLPTIYAITPTYSRPVQKAELTRLANTFRQVAQLHWILVEDRATRSELVSSFLARAGLPNTHLHVPTPRRYKRPWLPRATEQRNAGLAWLRQRHQHQSAQPGVLFFADDDNTYSLELFQEMRTTRKVSVWPVGLVGGRRYERPLVKNGKVVGWYTGWREDRPFAIDMAGFAVSLQVILSNPKAVFKRRGSQPGMQESDFLKQITTVEELEPKASNCTKVLVWHTRTEKVNLANEPKYHLDTVNIEV

SEQ ID NO: 41 - DNA, 1599 bp, Mus musculus
  CDS (175)..(1182)
gaagtggtcccaggtgtcgctcagagtcccgcgttcccgggaactagtaagggagcctggggaaggcggggcgcggcccgggcggccggggcggggccggcgggagggcggagggccgggcggacctgtccctgccggtcccggtggaccccgcctgccccggtggcaccggccatgaagctgaagctgaagaacgtgtttcttgcctacttcctggtgtcgatcgccggcctcctctacgctctggtgcagctcggccagccttgcgactgcctccctccgcttcgagctgcagctgagcagcttcggcagaaggacctgaggatatcccagttgcaagctgatctccgtcgcccaccccctgtcccagcccagccccctgaacctgaggccctgcctactatctatgtcattacccccacctacgccaggctggtacaaaaggcagagctggttcggctgtcccagaccctgagcttggtgccccgtctacactggctgctagtggaggacgctgagagccctaccccgctggtctcggggctgttggccgcctctggtctcctctttacacacctggctgtccttacccccaaggctcaacggcttagggaaggtgagccaggctgggtccggccccgaggagtggaacagcgcaataaggccctcgactggctccgaggaaaagggggtgctgttgggggggagaaggatccaccgccaccagggacccaaggagtcgtgtattttgctgacgatgacaacacctacagccgggagctctttaaggagatgcgttggactcgcggtgtctcagtgtggcctgtggggctggtgggtggcctgcgatttgaaggtcctcaggtacaggatggccgcgttgtgggtttccacacagcatgggaacccaacaggccctttcccttggacatggcgggatttgcggttgccctgcccttgctattggctaagcccaatgcccagtttgatgctactgcaccccggggccacctggaaagtagtctcctgagccaccttgtagatcccaaggacctggagccacgggctgccaattgtactcaggtactggtatggcacacccggacagagaaacctaagatgaagcaggaggagcagctacaacggcagggccagggctcagacccagccattgaggtgtgatggcaacctcaccctgacttctaccttattttaggcacataccttgtgggactgggctccaggccagcccaggatgtggttttttttttaaagacctgacccctgagaaccagaggcagcccctccaaccacagggtgtgtcccagttgcttccctcccttcccccagcctgccatgtggcactgcccacatgttggggacaagcagcttctctagtgagccagatcagccccatctggggcggagcaggacagatggactcagaaaggagggtagctgtgggaaaagggtaacttattggggacaagcatgcagtggggggctagaggagctgggctggaccctccccacctgagcatgcgatccccttcctacctctagaataaagaatctcaacccggaaaaaaaaaaaaaaaa

SEQ ID NO: 42 - protein, 335 aa, Mus musculus
MKLKLKNVFLAYFLVSIAGLLYALVQLGQPCDCLPPLRAAAEQLRQKDLRISQLQADLRRPPPVPAQPPEPEALPTIYVITPTYARLVQKAELVRLSQTLSLVPRLHWLLVEDAESPTPLVSGLLAASGLLFTHLAVLTPKAQRLREGEPGWVRPRGVEQRNKALDWLRGKGGAVGGEKDPPPPGTQGVVYFADDDNTYSRELFKEMRWTRGVSVWPVGLVGGLRFEGPQVQDGRVVGFHTAWEPNRPFPLDMAGFAVALPLLLAKPNAQFDATAPRGHLESSLLSHLVDPKDLEPRAANCTQVLVWHTRTEKPKMKQEEQLQRQGQGSDPAIEV

SEQ ID NO: 43 - DNA, 2974 bp, Mus musculus
  CDS (197)..(2203)
gataggggccccaggcttcccacagcatggagacacagggagtgtggagggtgccccccagcccacggacaacaccttcactccgaagtgtgagattgtgggcaaggacgcactgtcagcactggcccgggccagcaccaagcagtgtcaacaggagatcgctaatgtagtgtgcctgcaccaagctgggaacctaatgcccaagtctgtgccccggcactgccagctggctggcaagatgagccctggcgtccaatgggaagagatccgggcccagcagcctgttggtggccctccagtacgcatcgcctacatgctggtggttcacggccgtgctatccgccagctgaagcgtcttctgaaggccgtctaccatgagcaacacttcttttatatacatgtggacaagcgttccaactacctgtaccgggaggtagtggaactggcccagcactacgagaatgtgcgggtgacaccttggcgcatggtcaccatctggggcggggccagccttctgaggatgtatctgaggagcatgaaggaccttctggaaattcctggctggacctgggacttcttcatcaacctgagtgccactgactatccaacgaggacgaatgaggagctggtagcattcttatccaagaaccgggacaagaatttcctcaagtcacatgggcgagacaattccaggttcattaagaagcaaggcctggaccggcttttccatgagtgtgattcccacatgtggcgcctgggtgaacggcagatcccggcaggcattgtggtggatggtggctctgactggtttgtgctgacacgcagctttgtggaatatgtggtgtatacagatgaccccctcgtggcccagcttcgccagttctatacatacacattgcttccggctgagtcattcttccacacagtgctggagaacagcccggcctgtgcgagcctggtggacaacaacctgcgggtcaccaactggaaccgcaagctgggctgcaaatgccagtacaagcacatcgtggactggtgcggctgctcccccaacgacttcaagccacaggacttcctgaggcttcagcaagtctccagacccaccttctttgcccggaagttcgagtcgactgtgaaccaggaagtcctggaaattttggacttccatctatatggcagctacccacccagcacaccagccctcaaagcctactgggagaacatctacgacgtggccgatggccctggcggactcagcgacgtcctactcacagcctacacagcctttgcccgtctcagcctgcgtcatgctgccactgctgtatccccactggccactgcagtctgcaggtttgagcccagggggttgccgtccagcgtgcacctgtatttctatgacgaccatttccagggctacctggtgacgcaggcggtgcagccctcagcccaggggccagcagagacacttgagatgtggctgatgccccagaggtcgctgaagctgttggggcacagtgaccaggccagccggctccagagtctggaggttggtactgagtgggaccccaaagaacgtctcttccggaactttgggggcctgctgggaccactggatgagcctgtagccatgcagcgctgggcccggggccccaacctcacagccactgtggtctggattgaccccacctatgttgtggccacatcgtatgacatcacggtagatgcggataccgaagtcacgcagtacaagcctccactgagcctgccactgcggccgggagcctggactgtccgcttgcttcagttctgggaacccctgggtgagacccgcttcctcgtgctgccgttgacgttcaaccgcaaactacctctcaggaaagatgatgccagctggctgcatgcgggaccaccccacaacgaatacatggagcagagtttccagggcctaagtggcatcctgaatctgcctcaggcagaggccctggaggaggctgcccggaggcacacagagctcacgggttctgcacttgaggcctggacagatggggaactgagcaatttctggtctgtggcaggattgtgtgccataggaccttctgcttgtccctccctggagctctgcagactgaccagctggagctctctgtctcctgaccccaagtcagagctggggcctgtcaaagctgacgggcgactcaggtagcaggaccccagccagcacaacccggaggagccggggaattgcaccttacagacaacggagggacatctctcccacagacaagaaccagaggcccaggcagtttgcccatttgagccaacaggaacctacactgagggcagggaaggtgggtgcagtactccctactgctaacggccctgcaagagaccagcggtccactggagccatgaggaggatcttcctctgctcctcgttagtttatacttttttaataattaaaatgagtggcagagggaggacgatgtgcaatagggctcagagcagccccaggatatgggccatggccatggggagcaccacagagcatatcggtttctgctctggggaggcatgggctgcctccctctccagagcctccatccccagcctgcatctgaactgcgcactgctgaggctctgaggcccggaggacaggtgatctgtgggggctactgtggagttggactttagtgagcaggcctctcctctgggagccagggagccctagagcagagctggggccggctctcgggcttccctaggaaatgcgcgccatcagggcctgctgagatccatgaagtttccctactggaggaggaagcccactggcggcacaggaaggctgtctgtcagagctctcacaagagtcgagttgtgcctgaagctcagtgcgaagtctgaaatatgcaacagaggaaatatatctctatctctccaaaaaaaaaaaaaaaaaaaaaaaaaaaaaaaaaaaaa

SEQ ID NO: 44 - protein, 668 aa, Mus musculus
MPKSVPRHCQLAGKMSPGVQWEEIRAQQPVGGPPVRIAYMLVVHGRAIRQLKRLLKAVYHEQHFFYIHVDKRSNYLYREVVELAQHYENVRVTPWRMVTIWGGASLLRMYLRSMKDLLEIPGWTWDFFINLSATDYPTRTNEELVAFLSKNRDKNFLKSHGRDNSRFIKKQGLDRLFHECDSHMWRLGERQIPAGIVVDGGSDWFVLTRSFVEYVVYTDDPLVAQLRQFYTYTLLPAESFFHTVLENSPACASLVDNNLRVTNWNRKLGCKCQYKHIVDWCGCSPNDFKPQDFLRLQQVSRPTFFARKFESTVNQEVLEILDFHLYGSYPPSTPALKAYWENIYDVADGPGGLSDVLLTAYTAFARLSLRHAATAVSPLATAVCRFEPRGLPSSVHLYFYDDHFQGYLVTQAVQPSAQGPAETLEMWLMPQRSLKLLGHSDQASRLQSLEVGTEWDPKERLFRNFGGLLGPLDEPVAMQRWARGPNLTATVVWIDPTYVVATSYDITVDADTEVTQYKPPLSLPLRPGAWTVRLLQFWEPLGETRFLVLPLTFNRKLPLRKDDASWLHAGPPHNEYMEQSFQGLSGILNLPQAEALEEAARRHTELTGSALEAWTDGELSNFWSVAGLCAIGPSACPSLELCRLTSWSSLSPDPKSELGPVKADGRLR

SEQ ID NO: 45 - DNA, 5532 bp, Mus musculus
  CDS (369)..(1427)
ccagtgcacggcggcggcggcaccttccttctcgcatcccggtcgcccggcccgtgaggaggcagcggcggccgcaggcgccagcagaggaggcggcggagcatcgagcagaggcgaggcggaccggcaggagcgcgcggccggagccacgcatcctcacacttgcacaccaactcctgcgcctttcaccctctagaccagagtcggggcctcggagagcctcggtgaagctagcgggagccagctccggagcccagccagctctgttctgagcctccccggtgctggtacccccgcggcggggtccccgcttctgcgctcgggacgcgcctcccggacagccgggtccccgcggccaggacaaagccatgaagccggcgctgctggaagtgatgaggatgaacagaatttgccggatggtgctggccacttgcttcggatcctttatcttggtcatcttctatttccaaagtatgttgcacccagtcatgcggaggaaccccttcggtgtggacatctgctgccggaagggatcgagaagtcccctgcaggagctctacaatcccatccagctggagctatccaacactgccatcctgcaccagatgagacgggaccaggtgacagacacctgccgggccaacagtgccatgagccgcaagcgcagggtgctgacccccaacgacctgaagcacctggtggtggatgaggaccacgaactcatctactgctatgtgcccaaggtagcgtgcaccaactggaagaggctcatgatggtcctgagtggccggggcaagtacagcgatcccatggagatcccagccaacgaagcccacgtgtcggccaacctgaagacccttaaccagtacagcatcccagagatcaaccaccgcttgaaaagctacatgaagttcctgttcgtgcgggaacccttcgagaggctggtgtctgcctaccgcaacaagttcacgcagaagtacaacacctccttccacaagcgctacggcaccaagatcatccgacgccagcggaagaacgccacgcaggaggccctgcgcaagggggacgatgtcaagttcgaggagttcgtggcctacctcatcgacccccacacccagcgggaggagcccttcaacgagcactggcagacggtctactctctctgccacccgtgccacatccactacgacctcgtgggcaagtatgagacactggaggaggactccaattacgtactgcagctggccggagtgagcggctacctgaagttccccacctatgcaaagtccacccgaactaccgacgagatgaccacggagttcttccagaacatcagcgccgagcaccagacacagctgtacgaagtctacaaactggactttttaatgttcaactactcagtgccaaactacctgaagttggattagagggctgcggaggggagggggaggggtggttgggggagagggagagaatcctgctttttaatttaagatttttatttgtcaaaagaattctatggagactgggttattttgtaagttaatatgtcttcgggggagatgctgcgagcggcatggttaagaattatttaaaaattctccacggggaaggacagctgtctttgcaggggagcggggtggaatatccctgtttttagaagtggatactgcaacactgtctccaaggtgtccttgtgttctggtgaagtccacaaactgcattccataaagtctaatgaatcttatttatagttatttaaacgtggtctgtggcagcagcctgctcctgtctgtgcagaggagagtacagtctgtgcttcgctggctatggctcattgggggaggggagggggttatctcccccactgaccgtgtgtagaatcccatggtgacagctgcgggatgtgtgcctgtggccattcaagaaaccctgctagaaagaagtcaattgtccttgcactaagaaacagggatgagactggttcaaaccgattgctagcaaccccaaagtcccttgtagtaaagaaatgcaaatttttttaacccaagaagagagcagatgatagcgatttctctttaccgaaattagcaggctataagaataggctttctgtggaaatctttaagtggcactttccagtgctagcgaaggcagaataatggttgttttctagtcaataagtattgagcatctactattctccaggcttgactgcagatagcacattgagtgaaaccaggagccttggccttcagcaagctttatgtgaacacagggcatccagctcaacagtgaaccaacacccccatccctttggtcctcacacagactttagactattctcaagtggtccagagggtccaggaccctagcaaatttgcgtatttctacagttcaaatttgaaagccacacatttggatcaggccacttaactagctaagtagttcattgcccaacacaagaaatgacagaaacccttttgatcatgtatatctagtgcagtgtcttcatagtagaagcctataagccacaatattctccaagcgcaaggatggaggactgctgtgattgattagtgatgtctgccttgactaaggaaagggaggaacatttctcaagggcctcctactgtgatctccagtccacatcttgtcctgcctgccacttaaaacagtgcactgcatacatttggagactggcggagttcaagtgtgaaggtcaaaggtggagtgttggtacccacaggcacatgcccaccctccatgtcctgcattggcagagcatccaggacctccctctcctccagaaggttggaaatttgctctttcctgttccttgctgatcaatgcccaaacctattcctcatttctagttgtttctgatactttgcggttgacatttgagccacattcccatgacagccagtgtccgggtacgtagcagatgctgtacatagttctgatgataagggagctggtaggctgtaaattttgctttttgtgtttttgtggggttttgtttgttttgatgcagtatagagtgaagggggtaagaatattctgaaaaacataaaattgagtaatttattcacagaaactattaaaatggtattgttaggctcacaaggaggctgggggcagcggtaactgctacactgtctcgcagaggaaattattttatttaatgtgagagaaatgtgagccagagtggctgcctgaggctatttataaaacgcaggtaccttccccaatttctctcccctcgcctttaataaatagatttatgtcatgtgtgtgaccaagaacagtgacagatcttggctgtccctggaatagagaggcagtcatggtgctcaggccctgggcgtggtgtagctgatgcatgtcccaccgagcccaggctctccaaggcatcagtttgtggtcacatatccttagaaaacaaaattaagtcaggttcctgtctgcaggagttttcagagccttgaatacatagtctgccttgtgaaactgggagaatgtagcatttctgggctgcatgacctaggtatcctgtatggcagaccatgtcttagctggtttttcacacagtagccttcagtgagggtgatctagcctgtctgaggtctgcaaagattgacagctgcttcctagatccttccacaaatcagcagaaacagtgtccatctgggaactttatgagagtgttgctttacctagttggggtgtcttcatctggatactgccccagagagtgatcccgtccctccaagcacagcggagggaagcctagggtgttatggagtggggcaggtggcttaaggacaccttaaaggccacttggtggggataatggcctcgtggggatactcctcacttccacccacagtttggaactgttcactgtgtgcttcaggagttttaaatggagattcgtgttgatttccatggtccgcatctgtttgtcttccgtctgtggtccagtgctgtgtgcagatggtacttactccctttctctctcttacagggagaaagactgtccttctggggagagtgtatcaatacgcttccagaatgagggagtctttctcctgtcttgtgttcacacttcctagtgacaattttcaaaatatgtatcggcttgactgtttattgtagagtgtagaaaggtttctgtgttcctggctaaagacatccattggtgaaatgtgcttggaaatccaagtgttttctactgaggaaaaaaaaatgttggaaaagctttctcctgcatgcctcttgtgcttagctagaaaggggagctgccagcgtcccagctctgagccactgttcaaaggtgcagctgtgttttaggactaggtacaggcagagtgataaggacatgcctgcttagtgtactcatttatcgagaaagtttagactatgagccacaggggtccctgcctgcagtccctatagcctccccctgccccagtgcctttctcagctggtctgggaatcagggcagcccattgaggggggctcagacaaccctttcttcctggttctgggagtaaggctttctctgcagccacaaggagaacatgggaaccagaaacttctctttggtcagcccttctgagcacacaggtttagcccatgctccaagaatgctcctgagcattgtataagcccctaggactcttagctcatgtgataatgattgcattactatcctgctttaactgacaaaaccacagagaattttgcttgctttcctactctcttcctctgcccaggcctcctccatacgtatacatacacacatgggcatgtatacacacacatgcacaatagacatgcacacacacatgcacacatgggtatacatatacacacatgcacaatagacatgcatatatacatatacatgcacacattgataggcatacacacatgcacagtagacatgcacacatgggtatacatagacacacatgcacagacatgcatacacacacatgcacacataaatgtgcacacacacatacacaaattgcccaagcctgtgcactcctggtacccatgtcccatcctggcccacctctttgtcagcaggactgactccatcactccctcaggttttgcccaccagcaccccccagctttcccccagcagagctaaacagcccctctgaatagacaacaggaccttctagaaacagctgaacccttggacagcagcaggcagattttgatctgctctcatcaggtacaaaaggtggtatccgtattctcttctgagcatgctcagtaagcatatggacatagaatttaacatctctgtggagtgtgtgttttttacatatttgtatgcagtcgaggagggcctgttgtagaattctctccctgtatcttactatactgttaaagaagctgaattctatgttgccaacagatgcgtgaaatgttcctccaggaaaagccattcaagcctgattatttttctaagtaacttcaattaaattgaagaagaaaaaaaaaaaaaaa

SEQ ID NO: 46 - protein, 352 aa, Mus musculus
MKPALLEVMRMNRICRMVLATCFGSFILVIFYFQSMLHPVMRRNPFGVDICCRKGSRSPLQELYNPIQLELSNTAILHQMRRDQVTDTCRANSAMSRKRRVLTPNDLKHLVVDEDHELIYCYVPKVACTNWKRLMMVLSGRGKYSDPMEIPANEAHVSANLKTLNQYSIPEINHRLKSYMKFLFVREPFERLVSAYRNKFTQKYNTSFHKRYGTKIIRRQRKNATQEALRKGDDVKFEEFVAYLIDPHTQREEPFNEHWQTVYSLCHPCHIHYDLVGKYETLEEDSNYVLQLAGVSGYLKFPTYAKSTRTTDEMTTEFFQNISAEHQTQLYEVYKLDFLMFNYSVPNYLKLD

SEQ ID NO: 47 - DNA, 1785 bp, Mus musculus
  CDS (149)..(1408)
ggcgatttcggctgcagaatcagcatcaccagcaacagcagcagcggcggtgactgtggcgggcgctaggtccgtctcctagggaccatgtcccagctgtgcacaaggctgaagtgaagggccaggagtgggcccagcccagggcagcatgaccaagccgcggctcttccggctgtggctggtactagggtcggctctcatgatccttttgatcattgtatattgggacaacgtgggaaccgcccacttctatctgcacacgtctctctccaggccacacatcctagaaccccttcccacccagggattggtggaggagaacgtgttcacatctgacgtggatgagtttttggatactctccttagttctgacgcgaagcacaacgacctttccaggagaaaaactgagcagcccccggcgcccgcccccagcaagccagtcttgagccacatggaggagaacgtgagaggctacgactggtccactcatgatgcccatcagaaccctgaccgggacaggcagcaggccgagaggaggagcctgctgagagacttctgtgccaacgccagcctggcattccccaccaaggaccgctcttttgacgacatccccaactacgaactgaaccacctgatcgtggacgaccgccacggggtcatctactgctacgtgcccaaggtggcctgcaccaactggaagcgagtgatgatcgtgctgagcgagagcctgctggaccggggcagcccctaccgagaccccctggacatcccccgggaacacgtgcacaacaccagcacgcacctcaccttcaacaagttctggcgccgctacggaaagttctcccgtcacctcatgaaggtgaagctgaagaagtacaccaagttcctgttcgtgcgcgacccctttgtgcgcctcatctcagccttccgcagcaagttcgagctggagaacgaagagttttaccgcaagttcgcggtgcccatgctccgactgtacgccaaccacaccagcctgcccgcctcggtgagtgaggctttcagcgccgggctcaaggtctccttcgccaacttcatccagtacctcctagacccacacacggagaagctggcgcctttcaacgagcactggcgacaggtgtaccgcctctgccacccgtgccagatagactatgacttcgtggggaagctggagacgctcgatgaggacgctgcccagctcctgaggttcctcaaggtagactcccagctccacttcccccccagttatcggaacaggacggccagcagctgggaggaagactggtttgccaacatccccctggcatggaggcaacagctctataaactctacgaggccgactttgttctctttggctaccccaagccagaaaacctgctcagggactgagcccccagaagccctcacgctgcccccaacaaattgaatggctgtcccatgaggccgtcctttgaggatgggaccctgtggcctcctgggttctctcctggcttcctttgcttcctggtgtgacaggcagaggattccacgcccccccctcgcatctggagaccgtggtacagccaagaccgaagcacctcactctccagagttttgcgctccccacccccgcccttttgcaatctggatttgtttactccacagcctgtattcatggaacactgtgttaaatactgttttctaagattaatatatttcagatatatttaatacgaaagtgggaggaagctggagtaaagtgtggcgcccgcaaaaaaaaaaaaaaa

SEQ ID NO: 48 - protein, 419 aa, Mus musculus
MTKPRLFRLWLVLGSALMILLIIVYWDNVGTAHFYLHTSLSRPHILEPLPTQGLVEENVFTSDVDEFLDTLLSSDAKHNDLSRRKTEQPPAPAPSKPVLSHMEENVRGYDWSTHDAHQNPDRDRQQAERRSLLRDFCANASLAFPTKDRSFDDIPNYELNHLIVDDRHGVIYCYVPKVACTNWKRVMIVLSESLLDRGSPYRDPLDIPREHVHNTSTHLTFNKFWRRYGKFSRHLMKVKLKKYTKFLFVRDPFVRLISAFRSKFELENEEFYRKFAVPMLRLYANHTSLPASVSEAFSAGLKVSFANFIQYLLDPHTEKLAPFNEHWRQVYRLCHPCQIDYDFVGKLETLDEDAAQLLRFLKVDSQLHFPPSYRNRTASSWEEDWFANIPLAWRQQLYKLYEADFVLFGYPKPENLLRD

SEQ ID NO: 49 - DNA, 1728 bp, Mus musculus
  CDS (1)..(1116)
atgactgtcgcctgccacgcgtgccaggcacagcatgggaagacgctcctgttgcaggcggcccttgccggtggtggcaagtctgggtgctgcactcctgctcctgtgcgccctgcgtcccgggtaaccacaggaaaggatgcccaggacactgaatggcagggctccccaaaagcccttttgggggttccgacatttgaaaataaagctctgggctccagctggttcggtggagtgaggaagagtcccctacagctgttgcgtgacctggaccagggtccacgctccgcgatggccgaggtgcaccagcagcggcgtgagctgctgcgccgggcctgcagccgccacacgcgacgccaacgcctgctgcagccggaggacctgcgtcacgtgctggtggacgacgcgcaccggctgctgtactgctacgtgcctaaggtggcctgcaccaactggaagcgtgtgatgctggcgttgcgcggccgtggggatccaagcgcaatccctgcgcacgaggcgcatgcgcctggcctgctgccctcgctggccgactttgcgccggctgaggtcaactggcggctgcgcgactacctgacctttctcttcgtgcgggagcccttcgagcgcctggcgtcagcctaccgcaacaagctggcgcggccacacagcgcggccttccagcggcgctatggcacacgcatcgtgcgtcgcctacgaccacacgcgcagcccgatgcgctggcccgcggccacgacgtgcgcttcgccgagttcctggcctacctgctcgacccgcgcacgcgccgtcatgagcccttcaacgaacactgggagcgcgcacacgcgctgtgccatccgtgcctagtgcgctatgatgtagtgggcaagtttgagacgatagcagatgatgctgccttcgtgctggacctggtgggtgagcctgggctacgtttccctgctccaccgctgaggccagagaaggaccttacgcgtgagcaggcccggcgccttttccaggacatcagccccttctaccagcgtcgcctctttaacctctataagatggactttctgctcttcaattactctgccccttcctacctgcgactgcaataagggtgttgggtgcaatagagccagtggctgctgtgaccaggaggccaccaggaggtctggaacgaacctggttgtgtggattggagaccttatccagtgggcctgaccaagagtctggccactggtcacactcattccgactgggtagggtacaggttgctttaggtgaccataaccttgtcaggccgtttctgctgttagtttgatgtgtgtctcttcctcccactctgcagatgtcaggcttcttcctaggactccaggtttgtagttctttggtttggtttgagaggccatttctcagtcttgtctgtgtagaacctgtccgtggcatggtgctacaagacagaatgtcatggcttggttcagtgtggcctaaggtcttgtcagcatttactgcttaggagttaacatgagctgcctgccaccccagcagtcacaggatggtgagcactaccactccacatctacctggctgatctacctttgatctcagccttgcaagaggctggatcttcccctgtgtcagcaaaggccaagatgcaatactgtggcagcttttccagctcacttttattttttttgttgtttttttaaataaatatgttttgttac

SEQ ID NO: 50 - protein, 371 aa, Mus musculus
MTVACHACQAQHGKTLLLQAALAGGGKSGCCTPAPVRPASRVTTGKDAQDTEWQGSPKALLGVPTFENKALGSSWFGGVRKSPLQLLRDLDQGPRSAMAEVHQQRRELLRRACSRHTRRQRLLQPEDLRHVLVDDAHRLLYCYVPKVACTNWKRVMLALRGRGDPSAIPAHEAHAPGLLPSLADFAPAEVNWRLRDYLTFLFVREPFERLASAYRNKLARPHSAAFQRRYGTRIVRRLRPHAQPDALARGHDVRFAEFLAYLLDPRTRRHEPFNEHWERAHALCHPCLVRYDVVGKFETIADDAAFVLDLVGEPGLRFPAPPLRPEKDLTREQARRLFQDISPFYQRRLFNLYKMDFLLFNYSAPSYLRLQ

SEQ ID NO: 51 - DNA, 2073 bp, Mus musculus
  CDS (129)..(1259)
ccggccctggtccctgcctgcaccccgggagctggccacccactatccctcccctcccgagacctccagcccctgctgcagtcacctcccctgcagcctcgaggtcggcgaggtctggccgcagcaccatgtttccccgccctctgaccccactggctgccccgaaaagcgcggagaccctgggccgcacgccaaggcgggccccattgggccgggcccgggctgggctcggggggccgcccctgctgctgccgtccatgctgatgttcgctgtaatcgtggcctccagcggactgctgctcatgatcgagcgaggcatcctatcggagatgaaaccccttcccctgcaccctcccagccacaaaggcgcggcctggagcgggacagatcctaagcctagaggcctatccttggatgctggggactcggacttgcaagtgagggaggacatccgaaaccggaccttgagggccgtgtgcggacaaccaggcatgccccgggacccctgggacttgccggtgggacagcggcgcaccctgctgcgccacattctcgtaagtgaccgctaccgcttcctctactgctatgtccccaaagtggcctgctctaactggaaacgtgtgctgaaggtgctggctggcgtcctgaacaacgtggatgtccgcctcaagatggaccaccccagtgacttggtgtttctggcagacctgcggcctgaggagattcgctaccgtctgcagcactacttcaagttcctgtttgtgcgagaccccttggaacgcctcctgtctgcttaccgtaacaagtttggagagatccgagagtaccagcagcgatatggggccgaaattgtcaggcgctacagggctggagctggtcccagccctgcaggggacgatgtcaccttcccagagttcctgagatacctggtggatgaggatcctgaacatatgaatgagcattggatgcctgtgtaccacctgtgccaaccatgtgctgtgcactacgacttcgtgggttcctatgagaggctggaggctgatgccaaccaggtgctggagtgggtgcgggccccaccccatgtccggttcccagctcgccaggcctggtaccggccagccagcccagaaagtctgcattaccacttgtgcaatgttccacgggccctgcttcaagatgtgctacctaagtatatcctggacttctccctctttgcttacccactgcccaatgtcaccaaggaagcctgtcaccaatgacagtaggccagcaccttttggagtttgggtttaatgatatcagctttgggatgtctttcagagaaactcctggctctgggtggcttcctggtttctctaggtgtctccatatctcagtggtaaggactgtccttggaggtccttgtccacagtggctcagaggacagagctagaaaggaggcctgctgctttcactggtgaactgcctctcttaggggcctgtggtatccgtgtctgcagggcaccagtggttattaaagccatatgtttgatcgaaagactgacttcagccccctggctgctgggtctatgcagtccacctggtctgttgtaatttaacctgtggccaaatcccaaatatgacactagccaagcacatgatcatgcctaggaccaatggctgtgaccccctattcacccatcccatggacctcaggactggagtgagctgtggtgccttagaaatgaaatgtgtgcaattctactccagacttttacatttcctcctcttgctaggtctgaatcatttttctaaggaaagagaaacggaagtggggcccttacctcgaagctctaaagcccagcccctcaagcatccaaagacgcctgtgcctgacctcttcctagggctcctggagcatcttcaataagcctcccttccctacaaacctttggagactatgtgagactgtatggcccatatatctggctgtcacttgtctaatgcatttatttaaaatgtgtatattttaataggatccttgtaagggctgacttttaataaagctttttcatatacaaaaaaaaaaaaaaa

SEQ ID NO: 52 - protein, 376 aa, Mus musculus
MFPRPLTPLAAPKSAETLGRTPRRAPLGRARAGLGGPPLLLPSMLMFAVIVASSGLLLMIERGILSEMKPLPLHPPSHKGAAWSGTDPKPRGLSLDAGDSDLQVREDIRNRTLRAVCGQPGMPRDPWDLPVGQRRTLLRHILVSDRYRFLYCYVPKVACSNWKRVLKVLAGVLNNVDVRLKMDHPSDLVFLADLRPEEIRYRLQHYFKFLFVRDPLERLLSAYRNKFGEIREYQQRYGAEIVRRYRAGAGPSPAGDDVTFPEFLRYLVDEDPEHMNEHWMPVYHLCQPCAVHYDFVGSYERLEADANQVLEWVRAPPHVRFPARQAWYRPASPESLHYHLCNVPRALLQDVLPKYILDFSLFAYPLPNVTKEACHQ

SEQ ID NO: 53 - DNA, 6000 bp, Mus musculus
  CDS (496)..(1914)
cttagccactgaaccatctctccagcctctgttttggggtgtttgtttgagacagaatcaaaaggctggtctaaagtacatattgtagctaaagatggcttccaactcctgccttgcctccagagtgttagggtgtgacctttaaatgacctattaaagaaaccccaacaggtgtagagtacacctgtgatcccagctcttaggaggttggacagatggataaagaggtccaggtgtcccggactacatagagagaccctatctcaaaagagaggcgaagacttaggttttgagagtgggcctgaggtctctcttcaaaagttttatagaaagatgtctctgttccctgtctatgaacacggatggcctgagcacctgtctcttcacaggatcagagtgtcccccacctgaagagggctgattgggtcccaagctatgctcctgagctgagtgcctgcagccagtctgaggaactccatggcgccccctctccccatggagaaaggactcgctttgcctcaggatttccgggaccttgtacacagcctaaagattcgaggcagatacgtcttgttcctggcatttgtggtcatagtttttatcttcattgaaaaggaaaataaaatcatatccagggtctccgacaagctgaagcagatccctcattttgtggcagatgccaacagcactgacccagccctgctcttatcggagaatgcatctctcttgtccctgagcgagttggattccaccttttcccatctgcggagccgcctgcacaacctgagcctgcagctgggcgtggagccagcaatggagagccaggaggctggggcagagaagccatcccagcaggctggagcagggacccggcgccacgtgcttctcatggccaccacccgcacgggttcctcgttcgtgggcgagttcttcaaccagcagggcaatatcttctacctcttcgagccactgtggcacatcgagcgcaccgtgttcttccagcagcgaggcgccagcgcggctggttcagccttggtctaccgtgatgtcctcaagcagttgttgctatgcgacctgtatgtgctggagcccttcatcagccctccgcccgaggaccacttgactcagttcctgttccgccggggatccagccgttcactctgcgaggatccggtgtgcacacccttcgtcaagaaggtctttgagaagtaccactgcaggaaccgtcgctgcgggccactcaacgtgaccttggcgggcgaggcctgccgccgcaaggaccacgtggccctcaaggctgtgcgcatccgtcagctggagttcctgcagccgctagttgaggacccgaggttggatctacgagtcattcagctggtgcgcgacccccgggccgtgctggcttcacgcatagtggcctttgcgggcaagtatgagaactggaagaagtggctgtccgaggggcaggaccagctgagcgaggatgaggtgcagcgattgcggggcaactgtgagagcatccgcctgtctgcagagctgggcttgcggcagccagcctggctgcgcggtcgttacatgctggtgcgctatgaggatgtggcacgcaggccactgcagaaggcccgagagatgtacagctttgcgggcatccccttgaccccgcaggtggaggactggatccagaagaacacgcaggcgacacgcgacagcagcgatgtctactccactcagaaaaactcttctgagcagtttgagaagtggcgcttcagcatgcctttcaagctggcacaggtggtacaggctgcctgtggcccgaccatgcacctctttggctacaagttggccagggatgccgcctcactcaccaaccgctccatcagcctgctggaggagcggggcaccttctgggtcacgtagtgggggatgtctgggacccttggaactccttcttgtgaaaggctggccctgcttccctcacacccagcctggcagtgagacatagccctggcagaaagtcaaaatggggagcgatgatgggaacatagcccctggctgtagcggtagggccccctgccagctagactccccagagcagctacagctagggccttgggctcctctgaggacaccctgtctccttagtggtactggtcataaggtgtcctcaccctatgacctgcagtgtcccgagcaggtcaaggtaggttcctgtgtgtggacacacctccctagctctttctcacacagtctacacgaagcctgtaaaggcctgtaggttgtgtgggctggagtccaagtatttaacaaccagaaggggtgtagaccctctgcagcctctcagacctcctactgtattaagtgttaacctcttccgctctgagtcagagaagctgggatctggctgagtcctgggaaggaggaggacagcctagggtgaggaaggggacctttgaagctcctcagggaacagtggctgtgtaccagctcatagaaaatggtttgatcagctgttctagtcactcatgagtatcaatcagcctgtgtagagcaagacacacaaagtgcattgaaaacagacaaggccaggggtgtggctcagcggtaaagcccttgcctctctcatgcccagggccctaggtgtgattcttagcactataaatataagggaaaataagccatacatacgtgtgcagtacagagagagacagaaaagcgtaataaacattcccccctctcataagcacacgctctgcctctgtctctgtctctgtctctctctgtctctgtctctctctgtctctctctctcactctctctctctctctctctctctcacacacacacacacacacacacacacaaacacacacacacacactggtacagaaaagcttcccttctccactctttatgctcaccatgtttttaggatttttgtttgtttgttttgttttgggtttctatatatctaaacaaggagccttaaattacatctttttgggattacgtggcgagggggagcagcagggagcatttgcttctggtaaaaggatagaccagaaaggacttccccttggatgatcggagtcatgcagagcccccagggtcccacatgttctgtgtgaacttcatgtggaatgactcacagaagtgactaccttaggcgctcgctctgtagctcaatgtgcgtagagtttgtctattgtataggaagccctgggttctgtgccctgccccacaccatataggtatgatggcgtgtacccatatcccagcattcaggaaatcaaaggttggcggtcaaaagttcaaggtgcagctatgtatcttttgagatgtccgagtctataagctccctctactgagagctgctagtattccttctcaagaacaggttatttgaaaatctcttcttgtaagccccaacatttgggggaggggaggatgggggaactcttgactgagcagctaggtttccccaagcagaagttcttctctcccagtgcaagctgaggtttagtgccccggctagcctcggaactggctcagtgggggttggcacgctgacttcctggacaattgctgcatctgacactgtgagcgcccaggacaccggatgtgtagcatggatgtgggcctagcacaggtgtggaagaggacagtcaggacactcactgttggtgtttgttatccacctgttcagccgccgggtcccacacataggtcatctttcgtcactcaagccggcctctgtttttgattttaacaatccaagagcaagtgtgtaggggacaaagagacaccagctcttcatacttgatggagaccgggacaggatgctgcaggcaggctcgggagtgcattatcgtttcttctgagctcttcatccaggccatattcccttttctgcatcgcatctggggtgggaggggctgagctgggagtttccgtccttcttccctgtgggggtgggagatggggctcaaggtctccatctctcggctccctgaggacagaacccccacagtggacctttgggccttctcaggacattgacaatgttgtgtgcactgcaagttgactttatttattttgtaggaaaaagagatggtattctttagcaggatctgaaactgtaccctagtcaagaagtacaattaataacattattattaataacaattgggatgattcaaaggtcacacacacaccagaccacccaagaaccatgtaggagatgaggcggagccaggtcatggaagcccagaatgctacagagattctgtgggtctattattgatcagaaaatacaacatgggggggccttttctatggcataactcaggtgtctgcaaagacagagcagctcaggtgggtggataggcaggcagatggagccttgaatcacatcccctgaggctcagccagccagtgcgaatcctcaaggttcggaaagggacagtgtagcggcaaactactgggcatcttctctgatgcttagaggctcttaccaaggagcttgatggagaaagtggccatgttggtggcatgggacaactgctcacccaacttcaaagatcaaagggcacccaggtggcctatgacagtgacactctttcctaccattaggggtgtctgcccccactgaaagccatgaaatttctggccctaagggggtaggaaggacttaggagtagcagatggtttgatcccatcccccccacacacacacacacatccctcagctgtcttccacattagagccacttcagttgtccatggacttgtccctttgagacatcctggattttgaaggatagaaacatccccaagatggtctcgtgttaatcccacaacagaggcagaagggtcatactgagagagagagagagagagagagagagagagagaggagtgggggccagaccctcaagaagccaagtgggtctggtcaacctgtgcacatgagaaggagggaacatcactaaaatcagggcctgggctggtgtgttgttggtgagatcccgtggagtggctggctagatatggatgagttttctgagcatgctcacacacccccaacttcacattcttagaaatagcacaaccataatgccttacctcaaaaggatgaggcaaagcttgcaattaattcccagtgtctgaagacctccgggatcctttgagctgtgtgtccttgtcagtacatggggacaggctccttaggtattcctgacatagaggtaaggtgctgcccttgctgccttgcataagctgtgacaagcttcttactgggcacatagaacggctctgtcatctgcttccactaaatttgggcttggactttgcctcctgccaaatctccatctctgctggatagtctagtccctagggtctaaccaccaccctccacttcctggtgggtcctacaagcgctgtctttgtgccagtacccggatggtgtcctgcctcatggctaaatggtacaggacatcttcccagactgagtggtcatggcatgtgcatgtatgttcatgagtacatgcatggtgtgtgggggtggatgggtgcattctgttctgcttctggtgcagctacacagccacaacctctttctgtcattgaccttcttggtcttcttgtagccacagataatcttccaatgcccgattctgctgttctcatctgagagctgacaacccagcgctcagagtagagttcatgacctaggaaacccttctcctggtagcttactgaacttatttaattaaaaacgaacatataggggttggagagatggttcattggttaagagcaccggctgcttttgcaaaggacctgagttcaaatcccagcaaccatatggtggcttacaaccatctgtaatgagttctggtgccctcttctggcatgcaggtgtacatgcagatagagcactcctatgcataaaataaataaatcttttaaataaataaataaatgaacatgaaaaaaaaaaaaaaa

SEQ ID NO: 54 - protein, 472 aa, Mus musculus
MEKGLALPQDFRDLVHSLKIRGRYVLFLAFVVIVFIFIEKENKIISRVSDKLKQIPHFVADANSTDPALLLSENASLLSLSELDSTFSHLRSRLHNLSLQLGVEPAMESQEAGAEKPSQQAGAGTRRHVLLMATTRTGSSFVGEFFNQQGNIFYLFEPLWHIERTVFFQQRGASAAGSALVYRDVLKQLLLCDLYVLEPFISPPPEDHLTQFLFRRGSSRSLCEDPVCTPFVKKVFEKYHCRNRRCGPLNVTLAGEACRRKDHVALKAVRIRQLEFLQPLVEDPRLDLRVIQLVRDPRAVLASRIVAFAGKYENWKKWLSEGQDQLSEDEVQRLRGNCESIRLSAELGLRQPAWLRGRYMLVRYEDVARRPLQKAREMYSFAGIPLTPQVEDWIQKNTQATRDSSDVYSTQKNSSEQFEKWRFSMPFKLAQVVQAACGPTMHLFGYKLARDAASLTNRSISLLEERGTFWVT

SEQ ID NO: 55 - DNA, 2080 bp, Mus musculus
  CDS (147)..(1601)
caccgcccggctgctccactccgctccacccaacattgagggagacccgaagaggccggagccgaggactttgggctgggttttctggacagaaccagcaggcgcctactctgctctgggtggggagagtgaggattcggtgaactatgaagggccggcggcggcggcgccgaaagtattgcaagttcacgctgctcttggcgctgtacacgcttttgctacttcttgtcccctctgtactggacagccacagcgagcaggacaagggcaggaactgccccggcctgcagcgcagcttgggtgtgtggagcctggaggcggcggcggccggggaacgtgagcagggcgctgaggtgcggtccctggccgaaggaaacccggatcgatcccccgggtcccccggcaacctcagcgccgtcggtgaggcggtgacccaggaaaagcaacacatctatgtgcatgccacctggcgcaccggctcgtccttcttgggcgaactcttcaaccagcacccggacgttttctacttgtacgaccccatgtggcatctgtggcaggcactgtatccgggcgacgcggagagcctgcagggcgcactaagagacatgctgcgctccctcttccgctgtgatttctctgtgctgcgcctgtacgcgcagcctggggaccctggggagcgagcaccggactcggccaacctcaccacggccatgcttttccgctggcggaccaacaaggtcatctgctcgccgcctctgtgccccgccgcgccccgggcacgcgcggacgtgggactcgtcgaggacaaagcctgcgaaagtacctgcccgcccgtttcgctccgcgccctggaggccgagtgccgcaagtacccggtggtggtcatcaaagacgtgcggctactggacctgggagtgctggtccctctgctgcgtgacccaggcctcaacctaaaggtggtgcaactcttccgagaccctcgggccgtgcacaactcgcgcctcaagtcgaggcagggactgctgcgcgaaagcatccaggtgctgcgcacgcgccagaggggcgaccacttccaccgggtgctgctggcgcatggagtggatgcccgtccgggaggccaggcccgggctctgccctcggcgccacgcgctgatttcttcttaaccagcgcgcttgaggtgatctgtgaagcgtggcttcgcgacctgctattcacccgcggcgcgcccgcctggctgaggcgtcgctacctgcggctgcgttatgaggacctggtgtggcagccccaagcccagctgcgccgcctgctgcgcttctctgggttgcggacactcgccgcgcttgatgccttcgcattcaatatgacgcggggctcggcctacggcgccgatcgtcccttccacttgtctgcgcgggacgcccgagaggctgtgcacgcctggcgcgaacgtctgagccaagagcaggtgcgccaagtggaaaccgcctgcgcccctgccatgcgtctgcttgcctaccctcgaagtggggacgaacgcgacaggaagaccgtcagggaaggggagacaccactggagaccaaggccaattgggctgtgtaataccctgatccctgaaccctgccccggggcgtattcaggtcagtggccataaaaaggtgaactcagcatgctgcccccgcactggagaggctgcacggtggaggcgatctatcacactgtgagacactgggactgatttggtatcaactgctgtgccattctcctggtcaggagcatcacaagctgttaagtaatgacagacaccttggctgagatgaagtttccagaaaggaagtaacagtgcaatgtggatatttgtgaccacaacataggaaaagctgtacttcccaggctgaacttggctcagcttgagccatttcaacaaggcatcctcacaataatgaagagatgtgatctggtttcctttcacatcagccaagatgtctggacaaaaccatcaatgtgaataagggccaagtgcagttgtgtctctcttgattaaattacttcatattaaataaaaaaaaaaaaaaaaaaaa

SEQ ID NO: 56 - protein, 484 aa, Mus musculus
MKGRRRRRRKYCKFTLLLALYTLLLLLVPSVLDSHSEQDKGRNCPGLQRSLGVWSLEAAAAGEREQGAEVRSLAEGNPDRSPGSPGNLSAVGEAVTQEKQHIYVHATWRTGSSFLGELFNQHPDVFYLYDPMWHLWQALYPGDAESLQGALRDMLRSLFRCDFSVLRLYAQPGDPGERAPDSANLTTAMLFRWRTNKVICSPPLCPAAPRARADVGLVEDKACESTCPPVSLRALEAECRKYPVVVIKDVRLLDLGVLVPLLRDPGLNLKVVQLFRDPRAVHNSRLKSRQGLLRESIQVLRTRQRGDHFHRVLLAHGVDARPGGQARALPSAPRADFFLTSALEVICEAWLRDLLFTRGAPAWLRRRYLRLRYEDLVWQPQAQLRRLLRFSGLRTLAALDAFAFNMTRGSAYGADRPFHLSARDAREAVHAWRERLSQEQVRQVETACAPAMRLLAYPRSGDERDRKTVREGETPLETKANWAV

SEQ ID NO: 57 - DNA, 512 bp, Mus musculus
atgaaaggcatcttatggatgtgctcaaccttattactaacccatgcactacatcaagccaaaatggaaaccagcccacctgttaaaggctctctgtctggaaaagtggtcctaccttgtcatctttcaaccttacctaccttaccacccaattacaacacgagtgaatttctcagaatcaaatggtctaagatggaagtggacaaaaatggaaaagatataaaggagacgactgtcttggtggcccagaacggaaatatcaagattggtcaggactacaaggggcgagtgtccgtgcctacacatcccgatgatgtaggtgatgcttccctcaccatggtcaaactccgggctagtgatgcaggcgtctaccgatgtgatgtcatgtatgggattgaagacactcaggacaccatgtcactggctgtggatggtgttgtgtttcactacagggcagccaccagcaggtacactctgaactttgccgctgctcaacaggcttgtttggatat

SEQ ID NO: 58 - DNA, 536 bp, Mus musculus
acaaagccatgaagccggcgctgctggaagtgatgaggatgaacagaatttgccggatggtgctggccacttgcttcggatcctttatcttggtcatcttctatttccaaagtatgttgcacccagtcatgcggaggaaccccttcggtgtggacatctgctgccggaagggatcgagaagtcccctgcaggagctctacaatcccatccagctggagctatccaacactgccatcctgcaccagatgagacgggaccaggtgacagacacctgccgggccaacagtgccatgagccgcaagcgcagggtgctgacccccaacgacctgaagcacctggtggtggatgaggaccacgaactcatctactgctatgtgcccaaggtagcgtgcaccaactggaagaggctcatgatggtcctgagtggccggggcaagtacagcgatcccatggagatcccagccaacgaagcccacgtgtcggccaacctgaagacccttaaccagtacagcatcccagagatcaaccaccgcttg

SEQ ID NO: 59 - DNA, 660 bp, Mus musculus
gccaggagtgggcccagcccagggcagcatgaccaagccgcggctcttccggctgtggctggtactagggtcggctctcatgatccttttgatcattgtatattgggacaacgtgggaaccgcccacttctatctgcacacgtctctctccaggccacacatcctagaaccccttcccacccagggattggtggaggagaacgtgttcacatctgacgtggatgagtttttggatactctccttagttctgacgcgaagcacaacgacctttccaggagaaaaactgagcagcccccggcgcccgcccccagcaagccagtcttgagccacatggaggagaacgtgagaggctacgactggtccactcatgatgcccatcagaaccctgaccgggacaggcagcaggccgagaggaggagcctgctgagagacttctgtgccaacgccagcctggcattccccaccaaggaccgctcttttgacgacatccccaactacgaactgaaccacctgatcgtggacgaccgccacggggtcatctactgctacgtgcccaaggtggcctgcaccaactggaagcgagtgatgatcgtgctgagcgagagcctgctggaccggggcagcccctaccgagaccccctggacatcccccgggaaca

SEQ ID NO: 60 - DNA, 600 bp, Mus musculus
atgactgtcgcctgccacgcgtgccaggcacagcatgggaagacgctcctgttgcaggcggcccttgccggtggtggcaagtctgggtgctgcactcctgctcctgtgcgccctgcgtcccgggtaaccacaggaaaggatgcccaggacactgaatggcagggctccccaaaagcccttttgggggttccgacatttgaaaataaagctctgggctccagctggttcggtggagtgaggaagagtcccctacagctgttgcgtgacctggaccagatgtttggcagctgtgagctatgggtagtcagtggggagcggcagaaagtgggtccacgctccgcgatggccgaggtgcaccagcagcggcgtgagctgctgcgccgggcctgcagccgccacacgcgacgccaacgcctgctgcagccggaggacctgcgtcacgtgctggtggacgacgcgcaccggctgctgtactgctacgtgcctaaggtggcctgcaccaactggaagcgtgtgatgctggcgttgcgcggccgtggggatccaagcgcaatccctgcgcacgaggcgcatgcgcctggcctgctgccctcgctggcc

SEQ ID NO: 61 - DNA, 600 bp, Mus musculus
gcgccccctctccccatggagaaaggactcgctttgcctcaggatttccgggaccttgtacacagcctaaagattcgaggcagatacgtcttgttcctggcatttgtggtcatagtttttatcttcattgaaaaggaaaataaaatcatatccagggtctccgacaagctgaagcagatccctcattttgtggcagatgccaacagcactgacccagccctgctcttatcggagaatgcatctctcttgtccctgagcgagttggattccaccttttcccatctgcggagccgcctgcacaacctgagcctgcagctgggcgtggagccagcaatggagagccaggaggctggggcagagaagccatcccagcaggctggagcagggacccggcgccacgtgcttctcatggccaccacccgcacgggttcctcgttcgtgggcgagttcttcaaccagcagggcaatatcttctacctcttcgagccactgtggcacatcgagcgcaccgtgttcttccagcagcgaggcgccagcgcggctggttcagccttggtctaccgtgatgtcctcaagcagttgttgctatgcgacctgtat

SEQ ID NO: 62 - DNA, 720 bp, Mus musculus
tggggagagtgaggattcggtgaactatgaagggccggcggcggcggcgccgagagtattgcaagttcacgctgctcttggcgctgtacacgcttttgctacttcttgtcccctctgtactggacagccacagcgagcaggacaagggcaggaactgccccggcctgcagcgcagcttgggtgtgtggagcctggaggcggcggcggccggggaacgtgagcagggcgctgaggtgcggtccctggccgaaggaaacccggatcgatcccccgggtcccccggcaacctcagcgccgtcggtgaggcggtgacccaggaaaagcaacacatctatgtgcatgccacctggcgcaccggctcgtccttcttgggcgaactcttcaaccagcacccggacgttttctacttgtacgagcccatgtggcatctgtggcaggcactgtatccgggcgacgcggagagcctgcagggcgcactaagagacatgctgcgctccctcttccgctgtgatttctctgtgctgcgcctgtacgcgcagcctggggaccctggggagcgagcaccggactcggccaacctcaccacggccatgcttttccgctggcggaccaacaaggtcatctgctcgccgcctctgtgccccgccgcgccccgggcacgcgcggacgtgggactcgtcgaggacaaagcctgcgaaagtacctgcccgcccgtttcgc

SEQ ID NO: 63 - DNA, 600 bp, Mus musculus
accccacaactcggaacgatgcggctagcctgcatgttctcgtccatcctgctgtttggagctgcgggcctgctcctcttcatcagcctccaggaccctatagagctcagcccccagcaagttccaggtataaagttcagcatcaggccccagcaaccccagcatgatagccacttgaggatatccacagaaaagggcacacgagattcacccagcgggtcgccaagaggcctccagctgcaagcgcctgaccaacctcgacctcacccgaaggcagcgggatctcctttgcgcctccggcagcgcaggcggagactgctcatcaaaaagatgccagccgcagggactaaccaaggcaacaactcgtccgaaacctttatccagccgagaccccgcaccatggacagtcgttgggtcagcctgcaccagacccaacaggagcgcaagcgtgtgatgcgagaagcctgcgctaaatacagggccagcagcagccgcagagctgtcactccccgccacgtctcccgcatcttcgtggaggaccgccaccgtgtactgtactgtgaagtacccaaggcaggctgctccaactggaagagggtg

SEQ ID NO: 64 - DNA, 735 bp, Mus musculus
atatagtatctaggatatatgtagaagacaaacacaaaatcctgtactgtgaagtaccaaaagctggctgctctaattggaaaagaattctgatggtcctaaatggattggcttcctctgcatacaatatctcccatgatactgtgcactatgggaagcatctgaaaacactggatagttttgacttaaaaggagtacacatgcgtttgaatacatataccaaagctgtgtttgttagagatcccatggaaagattagtctccgcatttagggataaatttgagcatcccaatagttactaccatccggtgtttggaaaggcaattatcaagaaatatcgaccaaatgcctctgcagaagcattaaataatggatctggagtcaaattcaaagaattcgcctactatttgctggatgctcaccgtccagtaggaatggatattcactgggaaagagtcagcaaactgtgttatccgtgtttgatcaactatgactttgtagggaagtttgagaccttaggagaggatgccaattactttctacagttgattggtgctccaaaagagttgacatttccaaactttaaggataggcactcctctgatgaaagaaccaatgcccacgtggtaaggcagtatttaaaggacctgagcacagccgaaagacagctcatctatgacttctatcacttggactatttgatgtttaattacacaactccacatttgtaatttg

SEQ ID NO: 65 - DNA, 574 bp, Mus musculus
ccagaagccaagctcattgttatgctcagggatcctgtggagaggttgtactcagactatctctacttcgcaagttccaataaatctgcagatgacttccacgagaaagtgacggaggctctgcagctgtttgaaaattgcatgctggattattcactgcgcgcctgcgtctacaacaacaccctgaacaatgccatgcccgtgaggctccaggttggcctgtatgctgtgtacctcctggattggctgactgtcttcagtaaggagcagttcctcattctccgtctggaagaccacgcatccaatgtcaagtataccatgcacaaggtcttccagtttctaaacctggggcctctgagtgagaagcaggaagctctgatgaccaagagccctgcatccaacacacggcgtcctgaggatcgtagtctgggacccatgtggcccatcacccagaagattctgcgtgaattctatgggcctttcaacacaaggctggcacaggtcttggatgacgaagcatttgcctggaagacaacgtgagagctgagtcccttctgcagaagctgggcccact

SEQ ID NO: 66 - DNA, 1430 bp, Mus sp.
tgcgacttcatcaagtcggagccaaagaaatcttgaaagatgggaatgctagcaagagttgccctgggacttatcatcattgatgctgtattggctgcaccaactacagagctctttaactatgactcagaagtgtatgatgccatcttggaggacacagggaccttttataactatgaacacattcctgacaaccatgtagagaacgagaaagtgtctgagaggctttctgggaacagagagctcctgaccccaggcccacagctaggggacaaccaggatgaagacaaggatgaagaatctactcccaggctgatcgatggctcttccccacaggagccagaattcccggggcttctgggtccacacaccaacgaagactttccaacttgtctcctgtgtacttgtataagtaccactgtatactgtgatgaccatgaactggacgctattcctccactgcccaagaagactacatatttctattcacgttttaacagaattaaaaagatcaacaaaaatgactttgcaagcttaaatgatttaaaaaggatcgatctgacatcaaatttaatatcggagattgatgaagatgcattccgaaagctgcctcacctccaagagctggttctgcgtgacaataagataaagcagcttccagaattgccaaacactttgacatttattgatatcagcaacaatagactgggaagaaaaggcattaaacaagaagcttttaaagatatgtatgatctccaccatctctatatcaccgataacagcttggaccacatccctctaccactcccagaaagtctgcgggctcttcacctccagaataatgacattctggagatgcatgaagatactttctgcaatgttaaaaatttaacttatgttcgtaaggcattagaggatattcgactggatggaaaccctatcaacctcagcagaactcctcaagcctacatgtgtctacctcgtttgcccattgggagttttatctaatgttaggatgctaggcaaatatcagaattgtgatgctttctgtagccaagagaagcatatatctcttcagaagcaaactcagtatatttgagatgcgtttaaaacatgtggcacaatgatataaacaataagctaaaggtgttgagataaaataagagttcaataatttaattaaacatgattaggtataaattaacaaaatatatagacagtaagttaaataaaatgtatgccaactagtcatcaaatctcatcaagaattgtaggtgttcatgtcaagttaaatatgtaagaaataaaatattggtaatgaatactttagatacgttgctaagatgtagacattttaattaaacttctctagtctttattttcactcatgtatgtttcccacctgattcagttaagagcagaaaaaaata

SEQ ID NO: 67 - DNA, 27 bp, Mus musculus
atgaaaggcatcttatggatgtgctca

SEQ ID NO: 68 - DNA, 25 bp, Mus musculus
attactaacccatgcactacatcaa

SEQ ID NO: 69 - DNA, 25 bp, Mus musculus
ggcagccaccagcaggtacactctg

SEQ ID NO: 70 - DNA, 25 bp, Mus musculus
ctgctcaacaggcttgtttggatat

SEQ ID NO: 71 - DNA, 17 bp, Artificial
  An artificially synthesized primer sequence
gacgactgtcttggtgg

SEQ ID NO: 72 - DNA, 17 bp, Artificial
  An artificially synthesized primer sequence
atatccaaacaagcctg

SEQ ID NO: 73 - protein, 20 aa, Mus musculus
DRARSCAIVEDDGLQSAFTA

SEQ ID NO: 74 - DNA, 9120 bp, Mus musculus
tgtatatctgagtatctgtgtatgtgcatatatgtgtgtctatgtgtatgtgtgggtttgtgcatgtgtgtctgtgtgtacatgtctgtgtgtctctgtgtgtgtctgtgtatgtgtgtttacatgtgtgtgtgtgtttctgtgtggacgcatgtgtatgtgtctccgtgtgcctgcatgtgtgtttgtgtatgtgtgtaggcatgagtacatcatggtgtacatgtagaaattgagagagactcttgggtgtggttctcatctcccaccatatctgaagtaaatgtcttattcacaccactgtgcacatcagaaagctagccttgacctccaaggaactctgtctctgtctcctgtcttattgtaggtacctggggtttataagcctatgctgctgcatatggcttcacttgggttctggggatttgaacttaggtcctcacagttttagggcaagcattttattcactgaacaatctcccatgatccctagagtttattgtaggcaacttaacctttagtcgtcattgagatttagcattgatttcagttccagttgtattctgatgttgagcgctacagcagaggtacccagggactgctgattttctgtggccatgaatcctctagtgattaataaatttcatcataatgttctgtttatttctaaatgtccagacaactctaatttcaaattaatctcatcctaaacagcacaggataaaagttacagattgtcctatcttcattcatgtgatgcctcaaatcagatgtttaatcctttagtcagttatttgtgaaacggaacctaattacacacactcgcatgcgcgtgcgcgcacacacacacacacacacacacacacacacacacacacacacttgtagatttgcccacacagctgcttaagcaatttggagcaaatgtagaaatattatgaaatttcacataaaaatgggttttcctctgtcttcttgagaaatgtgtgagtttcaatatgacagtaatcttctggtgttggactataaccacttgaacccctgaggtactaagtgtgtacctactacgtgcctggattccatcatttccctacttatataagcttctccatcctgtagatcagcactgtgtaatggagtaacacaagtcattgaatcatgcaatcatttaatcaatttaattggcacttctgataagagaagtgggcacatatttagttgtgtttgcagcctgtgctatgtcatttttctctatatatactttaagatttttttcttttttccatttacggggggggggttattccatttaatttacatgcttaatgacatataatttgcatatcaggtgttcctttctttagagttaacttcttttaggaccattgtttataatggatgtatatcatccctaaattaaatcagccattgtctacttaacacaccagtttctctgatgtccctctggctcttcctcctctgaggttctagttataggaaagtctggcattttgatgtttttccatggcttcgtgggctctgatcatccacttacagtgtactttcatctcttgtctacaaactgcatatcatctattgatctgcgttcaaggctactacattttcatgcaattccactttgctgttaaaccttttctatgatccgtttttatacttataaacaatccaagtgttgtactctttgggtataaatgttctgtatttcacaggtcttaacttcttttcagactatacaaagaaagattttctattttttttatttgttgtatgcattttatgcatttagcactgggatttccataacaggcagttaagatcattgaatacatactttgtgtggtaaatataatacggcttatttgattctcccccttaaaatgctcccatcttctcagtcatgtcaggttgctatggagtccaccctgagcaccatgagtgtgatgctgtacagacactcctgatatgccttctgttatttgaactccacctgcaagttttcattcaagcatagctcctaatcccattcagtgtgcacttttcttaattgatttgctggtagagagctatatgtaagcatggcttagcatcaaattgaactttctcctctccctagcttctttccagtatattcttcctcaccttcatcagattttgaggtcttgagatctaaaccttcatgttacaatttgcagagataccaggatctctatcagacttgatgtgtctccatgtaattctagtatcagcctttcataagataattgcaaaatatagaaccccccctctctccttttgactattacagctttgtttctctcccaagttaatacagatgtatgcaaatcaactaagaaaagcaaacactgaatgggataaggagctatactcaaaagaaagcttgcagttggagttcaaataacacaagtggaaccaagagtatctgcacagcatcagactcactgtgctcaggatggaatccatagcaacctcccataactgagaggatgtgagccccttttaaagggaaaatcaaataagcagtattatattaaccacatgatgtgtatattcaatgtagattgggctgcaccgaagttatgattttagtaatgagtatttctccttgggttttcttctggaccaccctggttttccttttaaaaatagtaaaatagcagcaatgactgcttctcttaaaatctgaacaacacagagcacttcagcatcctttcaacctatctaaatccattgtagacctgcactgacttgctgtgagctattgtgtcacaactattattgtagctaaacaccttgaattttcaaagctgctctggactgtgtcttcttttagaattaaacttgctaaagatattggactctgagatgcttattcaccactttctataaaagagtaaggatgacaatgctttgagtagaagaagattgcatacgagggtaccagcatgtcattatgtccccagcagtcagtaaagtaaggatccagtgatggtagagttattatgtccattatggaactaatgtaaatgtttatcaatgactagaatggctgtaatcaccaatccttagacaagtgtgagtaacatcatgctcttttcttataataatagatagtcacacacaccatggtcttaaacattacattttgatttttctctttgtgtctgtcttttcgtacctctactccacttacctgagccttcagctagggctcctccaagacggcagtcacagtactagtgaggataattctcatatggagacctgaagcagaataaccttcttccaaggaaaccaaaatcctagtcaacatcttctccactctttggttatttatttttatttctatttctattttgccatttaagaatttttaaactcttttctgttttcttatatatattcttttttttacaacttttgatattctttatcaaatgatttatcccattgcctttcctcctgcagctatctgtgagttttagcatggatatttagaatacaattggaaattatgaaggtcgaataaaagttctctaagagccaggacctcactagcctagttagttgcttagtttctaatataaggcatggtttatctcctactgtatggcccttaaatccactagacatctgttggcttccaacagcatacaattgctgctattgtatgtttaggtatatatttttatgaaacactatattctgattggtgttcattcttcctatgaaggggaagattttattgcctgctcaaattcaagtcagtcctttttgagtagacaaaggttgtccaatcttgctaaacccttttcttttttgcttggggaatttctttagggaaaattctcaaccatgaaccaagcctagagattgataatagctgaaggcagagtagccagtcttagtaagggatgagagaaagactgaccaaagactgaataggaaatggttgatagcattcattgggactagacaataaagttagctaccccaacatgggcaaatggagaaaatggggagagtcatatggatatgtagggaggagaagaaaggaaggggtgagtttgaagtatggaagagcctttaaagcatttgtaagatttgtaagaaaaagcttttgagagacagagtattacctgccttcaaatcccttcaattactgtccctacaagacatacctgtgctcagctgctcttggaccacaagggggcctaagagcatgtttgtgtccctgtctttatacaggcttctcatctgtatttctcaaacgtaaatacaaatgggggagataacaaatgatatttctaagtggtacacaggtatgagaactgcacacaatctatttatatgtataatggccagagctctcagcagggaacagaggaagcctaggtaactcctgctccagagcatgtctgtatggagacctcagttcagcaactatgaaatctgtgttcaaagggtcatcactgggaacagtttctggaatagcagacatattcattctctctctctctctctctctctctctctctctctctctctctctctctctctctctctgtctgtctctgtctctctctgtctgtctgtctgtctctcccttctgtcaggggtatgcatacatgggctgggaattctatcttccctgcctgctgtctttggtgtctccctaatgtggtgccatgtttacttaacctgtctgaaatggaggataacctctggtctgataactgtcaagttactgaaatgtcagggaattgtgaatgaacccctctgaatgctgggcagatgttttgagcacgtgggtatccatagatttttttttcagcaggatttccttctttaatatattgtgggaacgaaacattgaagtttccaacactaagaaaagcaatgctctgggtttgcaaagaagtctccaaagctgtgggagaggcaatggctttgcctcacaaaggtgatcctgatttggattgggtgttcctcagtctccctcatcctttgtttgtggaccaaacctctgatcaaataatcagtatacagacaagtactgactgactcctgtcactgtttcccatgtaaattctcaaccatgtgtgtcagaattgtttgagtcatcaaagttccgtgatgctcaggatggtgaataccagaaactgaagtctgaaatccaatttggcctttctcgggcactgtaaactgagtgcaaatgtgcccagttccctcgtggcccatttatagcacaataacacagtgcttgcttacaaaatctagtcccgattaaagtatttatttaaagttgctttagacaaatatatttctaaatggaaggatgaaacagaaagaatattccaactgtacagtctatattttgtttctaaaaatttttattagaattctgacttttagaaaacaaatagatattcaaaaactacttctttaattaaataataacatcattatagacattctccagccatttagaagtagcatgattacaaagcattcatgttccctttgttttttgaaacagaagtatttatatgacaaataaaaagcaaaaatctcatatatgtgatagctatagatgcattgataggaattaaatttaatcaaaaagaacttttaatggcaataatgtggctactaacttcattgtttgtaataatgctgcaaatgtttaatgaaaactgttactaacaatacaggagaagattacagatatgattaggtcgtgttgatagaaatggaaagagaaaggtcactcactacctcagccacagaacttgaggaatgtgtgagggtttttgaggaaaaaagagaacaatgtaacatcacagcaacatcttaatctgaaagtagacgacaaaggtttcagagtaggggatcagaaatttaagtatgcagagaagcaaggcatatacggcttagacaatgtatgaagaacagcagcagacagcaagtgaaagaaatcacagtccacatggaaatcacacagaccacagtgtgcacacagagacagaaatcagcaagggacacacggcttccagttatgaaagttagacagcaaaatttaagttgtcccacctaaaacatgatgtatcgtctctcaaatggggaaggggaaggagcagcaaacatctggatatgtctaaaacctcacagagaacatctgggatctttgcatttctccctctgactttctcaatatgttagctacttctagatagtttcaataatggtctctaattgactatggagagacaggtctcatgtatgtgaacatccttaaccaggagatggaatgtgtttccttgtcactcattttctaaggaagtgaattccttctgcattgcccaggattggatgttacgtgaaactaaaaatcattgattaaaaagaaatgtcattaccacagttgggttgaatcaatcaagacttatcctagttacaggcatcacctccccttaagcacaaatactgagaacagctaagctcactttgaaatctgggatgaggaggctttaggtcaagcatctgacaatgtgtgtgtgtatgtgtgtgtgtgtgtgtgtgtgtagtatgattatatgtatatatgtagatgtgtgtatatatttatatgtatatatgtatatgtgagtatgtatatatgtatatgtgtgtatgtgtatgagtgtgtgtatgtatgactatgagtgtgtatgtattgtgcaagtgtgtgtatatatgtgtgtatatgtttatgtatatgtgtattgtatgggtgtgaatgtattatatatgtgtgtaggtacatatgtgtatatgtttatgcgtgtatatgtgtatgcattatatgtatgtgtgtatgtgtgtatgttttcatgtgtatgtagcatatgtatatgcatgtgtgtgtctgtgtgtgtaagcatgtatttactaagaaaaaatcagtttctctagctaatgagtcattgaagtgaagaaaccttacgctatcacattactatgtttccagcataaattgcaatatcagtaagacattccccatcataacttaatggacacttagattaggtcatgcagcagtaaattatgaaagcgtctatgctgagctgaggtgggaaatcgtctttcctgcctccttcctcctgttctttctcctaaaggccctcatcctctttctccccttcctttcttttaaaatattctatcattgatccaagctaactaacccttcccttgctcacatgtgacctgcccagaggaatctatttatagtcaacaataacaggccaaggacatgcgaacaaatgactttacttagccccacttggtgaatctattgggtgaataggtaaggggttatttataggagcttgactgactggcacagctgtatctctgaaaagtcccccaaagcatcatgggtgggaactaaggaaagccggaaatgctggagctggaatcccaacctgcagtggctacatgatgaatgtgtctcctttcctactaaatatttaagactctgggcaggctggttagtcttgtaaaagtcttgtgggtttctggagtcctgggaggctcactgtccctgcagaagcttgttattattgtatatgaaagaaagtgctcaacactgagtcacaaaagaagcccccgagaatcggtggcagaggtggaaatagaagaatccgatgcagttcccgaggcaagagttgatcctctccatggtgacaagaaaacaagagccacagaagcctgaaggctaagactatgacctgtgaggttctcctctcctaggacttgaatgcacttaactctgagggctagcagggcagagattcaggagcgttctcagaggttgtcccttctccttgtatgattctaggagaagctcagtgcataggcagaagttgcaaactctaacacattaacccacatcctgaattgttttatatcaattgttttataactacatgttttaacttttgttttggtttttgaattttgtttgtttgtttgttttctatttttcatatgtgtgcatgtgagccagaagacaggctaagggactctgttctctctttccagcatgtgggtttcagggatcaaagttaggtcccaaggcttcactgggcaacgggacccttagcctctgagtcatcttcttaatctttttctttgaaactttttcattacattgtgtcagtgcacacacacacacacacacacatgcatatacatccacatgcatatacatccacatgccatatgtcatggcacatgtgtggaaagaaatgggtgccttgaggaaatcagttttcttcttccatcacgtggatcttatgacttcaggtaacccatctctgctccccaagcatggctacaagcatttacctgccaagtcatctccctggctcccacattaatttataataatacaccctttcagcctagagagatgactcagtggttaagaactctggctgtgcttcagaaggttctgagttcaattcccagcagctataaggtggctcacaactatctctaatgttgttttctgtcctcttctggcatgcacttgtatgtgcagatagagtacttatatgcataaataaatataaatacattaataatcttttaaaaagaaggaaagattaaaagaacaatacaccctttcttttaagtaagaaaacatgcttagtatatagaataaatgctggtgttgaaaggtgaaattcagaagaaattataggttggaaatatttgaactcttttgtgtcaaaccaaatggtaaagatgatgtgataataaggtaaggttttcttcctacacatatacttgtttaataaccagtataagactatgagtgaacataactaaaaatacatgaaaaaaaactattttaaaaaaacatcactttcagatgactctggtgaaactcactgataaccagcaatctctttgatagaccagagtgtccagtctgaagttaattcttttgctttattctcaaacaatgttgcagaagtatgaaccatagtatcaagatacactgtccacatatgcacctggcctgacttgtgt